(19) United States
(12) Patent Application Publication
(10) Pub. No.: US 2026/0103720 A1
(43) Pub. Date: Apr. 16, 2026

(54) METHODS FOR HIGH DENSITY PLANTING OF RUBUS PLANTS

(71) Applicant: Pairwise Plants Services, Inc., Durham, NC (US)

(72) Inventors: Anabel Rivas, Watsonville, CA (US); Andrew Fister, Morrisville, NC (US)

(21) Appl. No.: 19/357,757

(22) Filed: Oct. 14, 2025

Related U.S. Application Data
(60) Provisional application No. 63/707,475, filed on Oct. 15, 2024.

(51) Int. Cl. C12N 15/82; C07K 14/435

(52) U.S. Cl. CPC C12N 15/8261; A01H 6/7499; C12N 15/8262; C12N 15/827

(57) ABSTRACT

This invention relates to methods for increasing yield per acre, harvesting efficiency, and the quality of the fruit produced by Rubus plants, or decreasing water and/or nitrogen use in fruit production in Rubus plants, comprising planting Rubus plants at a high density, wherein the Rubus plants comprise at least one mutation in an endogenous gene and exhibit a compact density and the ability to be planted at a high density.

### STATEMENT OF PRIORITY

This application claims the benefit, under 35 U.S.C. § 119 (e), of U.S. Provisional Application No. 63/707,475, filed on Oct. 15, 2024, the entire contents of which is incorporated by reference herein.

### STATEMENT REGARDING ELECTRONIC FILING OF A SEQUENCE LISTING

A Sequence Listing in XML format, entitled 1499-157_ST26.xml, 420,694 bytes in size, generated on Oct. 9, 2025 and filed herewith, is hereby incorporated by reference into the specification for its disclosures.

### FIELD OF THE INVENTION

This invention relates to methods for improving at least one agronomic trait in Rubus plants, comprising planting Rubus plants at a high density, wherein the Rubus plants exhibit a compact or determinate growth habit that enables planting at a high density and results in improved agronomic outcomes including yield, harvest efficiency, and resource-use efficiency. The compact phenotype may be conferred by modification of one or more endogenous genes regulating meristem identity, flowering behavior, plant architecture, or hormone signaling, including but not limited to TERMINAL FLOWER (TFL), CENTRORADIALIS (CEN), or functional equivalents thereof.

### BACKGROUND OF THE INVENTION

The current practices in cultivation of Rubus plants, such as blackberry, are characterized by wide plant spacing, extensive trellising, and labor-intensive pruning and harvesting. Such practices are increasingly unsuitable for sustainably increasing yields and optimizing resource utilization. These methods limit the number of plants per acre, thus capping the potential yield, and necessitate substantial labor and material inputs, driving up production costs. Additionally, the sprawling growth habit of traditional varieties leads to inefficient use of land and tunnel structures. The significant labor required for pruning and selective harvesting adds to the inefficiency and cost, making it challenging to scale production economically.

Yield in Rubus plants, such as blackberry, is often limited by the sprawling growth habit of traditional varieties, which require significant space and extensive maintenance, resulting in suboptimal land use. Harvesting berries from Rubus plants is labor-intensive, as the fruit is delicate and must be picked by hand to avoid damage. Workers often need to make multiple passes per week through the fields over several weeks, as the berries do not ripen simultaneously. This staggered ripening requires a skilled labor force to identify and pick the ripe berries while leaving the unripe ones to mature. The need for continuous monitoring and selective harvesting adds to the labor costs, making blackberry production a resource-intensive endeavor. Additionally, the manual nature of pruning, trellising, and harvesting underscores the need for innovations that can reduce labor inputs and improve efficiency in farming of Rubus plants.

The present invention overcomes these shortcomings in the farming of Rubus plants by providing improved methods for growing Rubus plants.

### SUMMARY OF THE INVENTION

The present invention provides a method of cultivating Rubus plants to improve agronomic performance, including increased yield per acre in Rubus plants, the method comprising planting Rubus plants at a high density, wherein the Rubus plants comprise at least one mutation in an endogenous TERMINAL FLOWER (TFL) gene, or other endogenous genes associated with plant architecture, and exhibit a compact growth habit that allows planting at a higher density and results in an increased in yield per acre and improved agronomic efficiency as compared to control Rubus plants.

The present invention further provides a method of improving at least one agronomic trait in Rubus plants, comprising planting Rubus plants at a high density, wherein the Rubus plants comprise at least one mutation in one or more endogenous genes that confer modifications in plant architecture and exhibit a compact growth habit resulting in improvement of at least one agronomic trait as compared to a control Rubus plant (devoid of the at least one mutation in the endogenous gene and does not exhibit compact growth).

The present invention further provides a method of improving at least one agronomic trait in a Rubus plant, comprising planting Rubus plants at a high density, wherein the Rubus plants comprise at least one mutation in an endogenous TERMINAL FLOWER (TFL) gene (e.g., one or more TFL genes) encoding a TFL polypeptide and exhibit a compact growth habit resulting in an improvement of at least one agronomic trait as compared to a control Rubus plant (devoid of the at least one mutation in the TFL gene and does not exhibit compact growth).

A further aspect provides a crop production system comprising: (a) a population of Rubus plants having a compact growth habit and comprising a mutation in one or more endogenous genes associated with plant architecture; and (b) the population of Rubus plants planted at a density of at least 4,000 plants per acre, wherein the system provides improved yield per unit area and reduced resource usage per pound of marketable fruit relative to a system comprising control Rubus plants.

crop production system comprising: (a) a population of Rubus plants comprising a mutation in an endogenous TERMINAL FLOWER (TFL) gene; and (b) the population of Rubus plants being planted at a density of at least 4,000 plants per acre, wherein the system provides improved yield per unit area and reduced resource usage per pound of marketable fruit relative to a system comprising control Rubus plants.

Another aspect of the invention provides a method of improving at least one agronomic trait in a Rubus plant, comprising planting compact Rubus plants at a high density, wherein the compact Rubus plants comprise at least one mutation in one or more endogenous genes associated with plant architecture and exhibit increased apical dominance (increased determinacy), reduced height, reduced cane length, increased cane number, reduced lateral branching and/or reduced time to flowering, thereby resulting in at least one improved agronomic trait as compared to a control Rubus plant (devoid of the at least one mutation in the endogenous gene and does not exhibit compact growth).

A further aspect provides a method of improving at least one agronomic trait in a Rubus plant, comprising planting compact Rubus plants at a high density, wherein the compact Rubus plants comprise at least one mutation in an endogenous TERMINAL FLOWER (TFL) gene and exhibit increased apical dominance (increased determinacy), reduced height, reduced cane length, increased cane number, reduced lateral branching and/or reduced time to flowering, thereby resulting in at least one improved agronomic trait as compared to a control Rubus plant (devoid of the at least one mutation in the endogenous TFL gene and does not exhibit compact growth).

Also provided are Rubus plants comprising in their genome one or more mutated endogenous genes as described herein, which may be planted at a higher density than a control Rubus plant not comprising the one or more mutated endogenous genes in its genome.

These and other aspects of the invention are set forth in more detail in the description of the invention below.

### BRIEF DESCRIPTION OF THE DRAWINGS

FIG. 1 shows yield (cumulative crates per acre per week (after planting)) for Rubus plants comprising a mutation in an endogenous TFL gene and control plants grown under standard density conditions and high density conditions.

FIG. 2 shows yield (crates per acre per week (after planting)) of Rubus plants comprising a mutation in an endogenous TFL gene and control plants grown under standard density conditions and high density conditions.

FIG. 3 shows percent of unmarketable fruit by weight for Rubus plants comprising a mutation in an endogenous TFL gene and control plants grown under standard density conditions and high density conditions.

FIG. 4 shows marketable and unmarketable fruit by pounds per acre for Rubus plants comprising a mutation in an endogenous TFL gene and control plants grown under standard density conditions and high density conditions.

FIG. 5 shows harvesting efficiency (harvested fruit weight (lbs) per person per minute) for Rubus plants comprising a mutation in an endogenous TFL gene and control plants grown under standard density conditions and high density conditions.

FIG. 6 shows the Brix seven days after harvest for the fruit Rubus plants comprising a mutation in an endogenous TFL gene and control plants grown under standard density conditions and high density conditions.

FIG. 7 shows the acidity seven days after harvest for the fruit Rubus plants comprising a mutation in an endogenous TFL gene and control plants grown under standard density conditions and high density conditions.

FIG. 8 shows the Brix:acidity ratio seven days after harvest for the fruit Rubus plants comprising a mutation in an endogenous TFL gene and control plants grown under standard density conditions and high density conditions.

FIG. 9 shows the flavor score (score of 1-9) seven days after harvest for the fruit Rubus plants comprising a mutation in an endogenous TFL gene and control plants grown under standard density conditions and high density conditions.

FIG. 10 shows nitrogen use per acre for Rubus plants comprising a mutation in an endogenous TFL gene and for control plants grown under standard density conditions and high density conditions.

FIG. 11 shows nitrogen use per plant for Rubus plants comprising a mutation in an endogenous TFL gene and for control plants grown under standard density conditions and high density conditions.

FIG. 12 shows pounds of marketable fruit harvested per pound of nitrogen applied for Rubus plants comprising a mutation in an endogenous TFL gene and for control plants grown under standard density conditions and high density conditions.

### BRIEF DESCRIPTION OF THE SEQUENCES

SEQ ID NOs:1-17 are exemplary Cas12a amino acid sequences useful with this invention.

SEQ ID NOs:18-20 are exemplary Cas12a nucleotide sequences useful with this invention.

SEQ ID NO:21-22 are exemplary regulatory sequences encoding a promoter and intron.

SEQ ID NOs:23-29 are exemplary cytosine deaminase sequences useful with this invention.

SEQ ID NOs:30-40 are exemplary adenine deaminase amino acid sequences useful with this invention.

SEQ ID NO:41 is an exemplary uracil-DNA glycosylase inhibitor (UGI) sequences useful with this invention.

SEQ ID NOs:42-44 provides an example of a protospacer adjacent motif position for a Type V CRISPR-Cas12a nuclease.

SEQ ID NOs:45-47 provide example peptide tags and affinity polypeptides useful with this invention.

SEQ ID NOs:48-58 provide example RNA recruiting motifs and corresponding affinity polypeptides useful with this invention.

SEQ ID NOs:59-60 are exemplary Cas9 polypeptide sequences useful with this invention.

SEQ ID NOs:61-71 are exemplary Cas9 polynucleotide sequences useful with this invention.

SEQ ID NOs:72, 106, 114, 126, 140, 162, 172, or 184 are example TFL genomic sequences useful with this invention.

SEQ ID NOs: 73, 107, 115, 127, 141, 153, 163, 173, or 185 are example TFL coding sequences (cds) useful with this invention.

SEQ ID NOs: 74, 108, 116, 128, 142, 154, 164, 174, or 186, or are example TFL polypeptide sequences useful with this invention.

SEQ ID NOs:75-105, 109-113, 117-125, 129-139, 143-152, 155-161, 165-171, 175-183, or 187-192 provide example portions or regions of TFL genomic sequences useful with this invention.

SEQ ID NOs:193-197 provide example portions or regions of a TFL polypeptide.

SEQ ID NOs:198-210 are example spacer sequences for CRISPR-Cas guides (Cas12) useful with this invention.

SEQ ID NOs:211-213 are example spacer sequences for CRISPR-Cas guides (Cas9) useful with this invention.

SEQ ID NO:214 and SEQ ID NO:215 are a TFL consensus genomic sequence and a TFL consensus coding sequence, respectively, from the blackberry line A.

SEQ ID NO:216 is a TFL consensus coding TFL polypeptide from the blackberry line A.

SEQ ID NOs:217-233 are example portions or regions of TFL genomic sequences from the blackberry line A useful with this invention.

SEQ ID NOs:247, 271-274 and 275 provide example portions or regions of a TFL polypeptide from the blackberry line A.

SEQ ID NO:234 and SEQ ID NO:235 are a TFL consensus genomic sequence and a TFL consensus coding sequence, respectively, from the blackberry line B.

SEQ ID NO:236 is a TFL consensus coding TFL polypeptide from the blackberry line B.

SEQ ID NOs:237-246 are example portions or regions of TFL genomic sequences from the blackberry line B useful with this invention.

SEQ ID NOs: 248, 271-274, and 276 provide example portions or regions of a TFL polypeptide from the blackberry line B.

SEQ ID NO:252 is a TFL consensus genomic sequence from the blackberry line C.

SEQ ID NO:253 is a TFL consensus coding TFL polypeptide from the blackberry line C.

SEQ ID NOs:254-270 are example portions or regions of TFL genomic sequences from the blackberry line C useful with this invention.

SEQ ID NOs: 247, 271-274, and 275 provide example portions or regions of a TFL polypeptide from the blackberry line C.

SEQ ID NOs:249-251 are example spacer sequences for CRISPR-Cas guides useful with this invention.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 96%, 97%, 98%, 99%, 99.5%, 99.6%, 99.7%, 99.8%, 99.9%, or 100% as compared to a control. In particular embodiments, the reduction can result in no or essentially no (i.e., an insignificant amount, e.g., less than about 10% or even 5%) detectable activity or amount. For example, “reduced time to flowering,” or “reduced time to initiation of flowering” means a reduction in the time to flower initiation (e.g., in a Rubus plant) of about 5% to about 95%, (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95%; e.g., about 5% to about 75%, about 5% to about 80%, about 5% to about 85%, about 5% to about 90%, about 5% to about 95%, about 15% to about 80%, about 15% to about 85%, about 25% to about 75%, about 25% to about 80%, about 25% to about 85%, about 25% to about 90%, about 25% to about 95%, about 50% to about 80%, about 50% to about 85%, about 75% to about 80%, about 75% to about 85%, about 75% to about 90%, about 75% to about 95%, about 80% to about 85%, about 80% to about 95%), or any range or value therein, as compared to a control plant not comprising the same mutation. A control plant is typically the same plant as the edited plant, but the control plant has not been similarly edited and therefore is devoid of the mutation. A control plant maybe an isogenic plant and/or a wild type plant, which is devoid of the mutation. Thus, a control plant can be the same breeding line, variety, or cultivar as the subject plant into which a mutation as described herein is introgressed, but the control breeding line, variety, or cultivar is devoid of the mutation. In some embodiments, a comparison between a plant of the invention and a control plant is made under the same growth conditions, e.g., the same environmental conditions (soil, hydration, light, heat, nutrients, and the like).

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or, for example, a functional untranslated RNA.

A “heterologous” or a “recombinant” nucleotide sequence is a nucleotide sequence not naturally associated with a host cell into which it is introduced, including non-naturally occurring multiple copies of a naturally occurring nucleotide sequence. A “heterologous” nucleotide/polypeptide may originate from a foreign species, or, if from the same species, is substantially modified from its native form in composition and/or genomic locus by deliberate human intervention.

A “native” or “wild type” nucleic acid, nucleotide sequence, polypeptide or amino acid sequence refers to a naturally occurring or endogenous nucleic acid, nucleotide sequence, polypeptide or amino acid sequence. In some contexts, a “wild type” nucleic acid is a nucleic acid that is not edited as described herein and can differ from an “endogenous” gene that may be edited as described herein (e.g., a mutated endogenous gene). In some contexts, a “wild type” nucleic acid (e.g., unedited) may be heterologous to the organism in which the wild type nucleic acid is found (e.g., a transgenic organism). As an example, a “wild type endogenous TFL gene” is a TFL gene that is naturally occurring in or endogenous to the reference organism, e.g., a plant in the Rubus family, and may be subject to modification as described herein, after which, such a modified endogenous gene is no longer wild type.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “allele” refers to one of two or more different nucleotides or nucleotide sequences that occur at a specific locus.

A “null allele” is a nonfunctional allele caused by a genetic mutation that results in a complete lack of production of the corresponding protein or produces a protein that is non-functional.

A “recessive mutation” is a mutation in a gene that produces a phenotype when homozygous but the phenotype is not observable when the locus is heterozygous.

A “dominant mutation” is a mutation in a gene that produces a mutant phenotype in the presence of a non-mutated copy of the gene. A dominant mutation may be a loss or a gain of function mutation, a hypomorphic mutation, a hypermorphic mutation or a weak loss of function or a weak gain of function.

A “dominant negative mutation” is a mutation that produces an altered gene product (e.g., having an aberrant function relative to wild type), which gene product adversely affects the function of the wild-type allele or gene product. For example, a “dominant negative mutation” may block a function of the wild type gene product. A dominant negative mutation may also be referred to as an “antimorphic mutation.”

A “semi-dominant mutation” refers to a mutation in which the penetrance of the phenotype in a heterozygous organism is less than that observed for a homozygous organism.

A “weak loss-of-function mutation” is a mutation that results in a gene product having partial function or reduced function (partially inactivated) as compared to the wildtype gene product.

A “hypomorphic mutation” is a mutation that results in a partial loss of gene function, which may occur through reduced expression (e.g., reduced protein and/or reduced RNA) or reduced functional performance (e.g., reduced activity), but not a complete loss of function/activity. A “hypomorphic” allele is a semi-functional allele caused by a genetic mutation that results in production of the corresponding protein that functions at anywhere between 1% and 99% of normal efficiency.

A “hypermorphic mutation” is a mutation that results in increased expression of the gene product and/or increased activity of the gene product.

As used herein, a “non-natural mutation” refers to a mutation that is generated though human intervention and differs from mutations found in the same gene that have occurred in nature (e.g., occurred naturally).

The terms “determinate” or “indeterminate” are used herein in reference to the growth habit of a plant shoot. An “indeterminate” shoot meristem refers to a shoot meristem that continues to grow (no defined end status). A “determinate” shoot meristem refers to a shoot meristem that grows to a fixed length or for fixed length of time. The term “determinate plant growth” refers to plant growth in which the main stem ends in an inflorescence or other reproductive structure (e.g., a bud) and stops continuing to elongate indefinitely with only branches from the main stem having further and similarly restricted growth, e.g., growth characterized by sequential flowering from the central or uppermost bud to the lateral or basal buds. The term “indeterminate plant growth” refers to plant growth in which the main stem continues to elongate indefinitely without being limited by a terminal inflorescence or other reproductive structure, e.g., growth characterized by sequential flowering from the lateral or basal buds to the central or uppermost buds, e.g., the growth of the axis of the plant is not limited by a reproductive structure.

A “locus” is a position on a chromosome where a gene or marker or allele is located. In some embodiments, a locus may encompass one or more nucleotides.

As used herein, the terms “desired allele,” “target allele” and/or “allele of interest” are used interchangeably to refer to an allele associated with a desired trait. In some embodiments, a desired allele may be associated with either an increase or a decrease (relative to a control) of or in a given trait, depending on the nature of the desired phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with increased yield under non-water stress conditions may be introgressed from a donor into a recurrent parent that does not comprise the marker and does not exhibit increased yield under non-water stress conditions. The resulting offspring could then be backcrossed one or more times and selected until the progeny possess the genetic marker(s) associated with increased yield under non-water stress conditions in the recurrent parent background.

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized, e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants may be grown, as well as plant parts that can be cultured into a whole plant (e.g., leaves, stems, buds, roots, pollen, cells, etc.).

As used herein, the terms “cultivar” and “variety” refer to a group of similar plants that by structural or genetic features and/or performance can be distinguished from other varieties within the same species.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term may refer to a plant or plant variety that is substantially homozygous throughout the entire genome or that is substantially homozygous with respect to a portion of the genome that is of particular interest.

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterologous” refers to a nucleotide/polypeptide that originates from a foreign species, or, if from the same species, is substantially modified from its native form in composition and/or genomic locus by deliberate human intervention.

As used herein a “control plant” means a plant that does not contain an edited gene as described herein (e.g., an edited TFL gene, or an edited gene of any one or more of CEN (CENTRORADIALIS), BRC1 (BRANCHED1), AP1 (APETALA1), FT (FLOWERING LOCUS T), GA20ox/GA3ox (GIBBERELLIN 20-OXIDASE, GIBBERELLIN 3-OXIDASE), GA2ox (GIBBERELLIN 2-OXIDASE), GID1 (GIBBERELLIN IN SENSITIVE DWARF1), DELLA (DELLA PROTEIN (GA SIGNALING REPRESSOR), SLY1/GID2 (SLEEPY1, GIBBERELLIN INSENSITIVE DWARF2), YUCCA (YUCCA FLAVIN MONOOXYGENASE-LIKE), PIN1/PIN3/PIN4 (PIN-FORMED 1 AUXIN TRANSPORTER, PIN3, PIN4), AUX1, BR1/BR2 (BR1, BR2), AUX/IAA (AUX, IAA), DWF4/CPD (DWARF4 STEROL C-22 HYDROXYLASE, CPD), BRI1/BAK1 (BRASSINOSTEROID INSENSITIVE 1, BAK1), BIN2, CCD7/CCD8/D27/MAX (CCD7, CCD8, D27, MAX), D14/MAX2 (DWARF14 α/β HYDROLASE, MORE AXILLARY GROWTH 2), CKX (CYTOKININ OXIDASE/DEHYDROGENASE), IPT (ISOPENTENYL TRANSFERASE), PIF4/PIF5 (PIF4, PIF5), PHYB, HFR1 or BFT (BROTHER OF TFL AND FT), or orthologues thereof) and further does not comprise a compact growth habit or the ability to be planted at a high density without compromising at least one agronomic trait such as yield, fruit quality, resource use efficiency and/or harvesting efficiency. Many methods for identifying endogenous genes and their orthologues are known in the art including, but not limited to, the use of sequence alignment tools including BLASTP, TBLASTN, or BLASTN, or equivalent alignment algorithms, or the use of syntenic analysis. Conservation of diagnostic motifs or catalytic domains characteristic of a given gene family may also be used to identify orthologues or sequences with functional equivalence. Other methods for identifying orthologues and the like are available and well-known in the art and may be used with this invention.

A control plant is used to identify and select a plant edited as described herein and as a comparator for high density planting of the edited plants. An example of a suitable control plant can be a plant of the parental line used to generate a plant comprising a mutated TFL gene(s), for example, a Rubus plant that is devoid of an edit in an endogenous TFL gene as described herein. A suitable control plant can also be a plant that contains recombinant nucleic acids that impart other traits, for example, a transgenic plant having enhanced herbicide tolerance. A suitable control plant can in some cases be a progeny of a heterozygous or hemizygous transgenic plant line that is devoid of the mutated TFL gene as described herein, known as a negative segregant, or a negative isogenic line.

An enhanced trait (e.g., improved yield trait) may include, for example, a compact growth habit as described herein. An enhanced trait (e.g., improved yield trait) may further include, for example, decreased days from planting to maturity (increased determinacy (earlier to stop growth and flower)), reduced plant height, increased apical dominance, reduced cane length, reduced lateral branching, increased number of canes, increased number of root branches, increased total root length, increased yield, increased nitrogen use efficiency, and increased water use efficiency as compared to a control plant.

As used herein a “trait” is a physiological, morphological, biochemical, or physical characteristic of a plant or particular plant material or cell. In some instances, this characteristic is visible to the human eye and can be measured mechanically, such as seed or plant size, weight, shape, form, length, height, growth rate and development stage, or can be measured by biochemical techniques, such as detecting the protein, starch, certain metabolites, or oil content of seed or leaves, or by observation of a metabolic or physiological process, for example, by measuring tolerance to water deprivation or particular salt or sugar concentrations, or by the measurement of the expression level of a gene or genes, for example, by employing Northern analysis, RT-PCR, microarray gene expression assays, or reporter gene expression systems, or by agricultural observations such as hyperosmotic stress tolerance or yield. However, any technique can be used to measure the amount of, the comparative level of, or the difference in any selected chemical compound or macromolecule in the transgenic plants.

As used herein an “enhanced trait” means a characteristic of a plant resulting from mutations in any one or more of the endogenous genes as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof). In some embodiments, and enhanced or improved agronomic trait may result from mutations in TFL genes as described herein. Such traits include, but are not limited to, an enhanced agronomic trait characterized by enhanced plant morphology, physiology, growth and development, yield, nutritional enhancement, disease or pest resistance, or environmental or chemical tolerance. In some embodiments, an enhanced trait/altered phenotype may be, for example, a compact growth habit as compared to a control plant, optionally without compromising yield, optionally wherein the yield is increased. In some embodiments, an enhanced trait/altered phenotype may be, for example, compact plant growth, optionally wherein the yield is increased, as compared to a control plant. “Yield” can be affected by many properties including without limitation, plant height, plant biomass, fruit number, efficiency of nutrient assimilation, resistance to biotic and abiotic stress, carbon assimilation, plant architecture, resistance to lodging, percent seed germination, seedling vigor, and juvenile traits. Yield can also be affected by growth rate (including growth rate in stressed conditions), flowering time and duration, fruit number and fruit size.

Also used herein, the term “trait modification” encompasses altering the naturally occurring trait by producing a detectable difference in a characteristic in a plant comprising a mutation in, for example, an endogenous TFL gene as described herein relative to a plant not comprising the mutation, such as a wild-type plant, or a negative segregant. In some cases, the trait modification can be evaluated quantitatively. For example, the trait modification can entail an increase or decrease in an observed trait characteristics or phenotype as compared to a control plant. It is known that there can be natural variations in a modified trait. Therefore, the trait modification observed entails a change of the normal distribution and magnitude of the trait characteristics or phenotype in the plants as compared to a control plant.

The present disclosure relates to Rubus plants with an improved economically relevant characteristic, for example increased yield per acre, increased harvesting efficiency, increased quality of the fruit produced by Rubus plants (increased Brix, increased Brix:acidity ratio), increase in marketable fruit, a decrease in unmarketable fruit, decreased water and/or nitrogen use in fruit production. More specifically, the present disclosure relates to Rubus plants having at least one mutation in an endogenous gene such as TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof, which confers compact plant growth and at least one improved agronomic trait on the Rubus plant, thus allowing the Rubus plants to be planted at a high density. Accordingly, and as an example, the present disclosure relates to a method of improving an agronomic trait in Rubus plants, comprising planting the Rubus plants at a high density, wherein the Rubus plants planted at a high density comprise at least one mutation in an endogenous TERMINAL FLOWER (TFL) gene (e.g., one or more TFL genes) encoding a TFL polypeptide, and exhibit a compact growth habit. The compact growth habit of the Rubus plants of this invention (e.g., comprising at least one mutation in an endogenous gene, for example, an endogenous TFL gene encoding a TFL polypeptide), which results in at least one improved agronomic trait is as compared to a control Rubus plant devoid of the at least one mutation in an endogenous gene.

Planting traditional Rubus plants, including blackberry plants, at high densities, such as, for example, 2 feet between plants and 5 feet between rows, poses several challenges environmentally, agriculturally and commercially. Blackberry plants are large and require ample sunlight, but dense planting can lead to canopy crowding, reducing the light penetration and fruit quality. Poor air circulation in tightly spaced plants increases the risk of fungal diseases like botrytis and powdery mildew, among others. The crowded conditions may also create ideal environments for pests to spread easily. Additionally, plants planted at a high density grow into each other (both above ground and below ground) and therefore the plants compete more intensely for water and nutrients (than those planted farther apart) (increased resource competition as compared to Rubus plants having compact growth conferred by editing at least one endogenous gene as described herein), leading to stunted growth and again, a higher susceptibility to pests and diseases. In addition, high density planting complicates maintenance, as it makes pruning, trellising, and harvesting more difficult, increasing labor inefficiency (increased resource utilization as compared to Rubus plants comprising a mutation in at least one endogenous gene as described herein and having compact growth). Over time, the competition for resources and the physical stress of overcrowding may reduce the longevity and productivity of the Rubus plants. Consequently, high density planting as described herein for the compact, edited plants is not considered to be commercially or agriculturally viable for Rubus plants not comprising a mutated endogenous gene as described herein and not having a compact growth habit. Instead, more generous spacing is typically recommended to ensure healthy, high-yielding plants. Thus, the Rubus plants comprising a mutation in an endogenous TFL gene or in an endogenous TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof, and having compact growth allows a grower to plant at a higher density without the above described negative consequences, providing greater overall yield without the increase in labor resources (e.g., increased harvesting efficiency) and inputs/resources needed (e.g., reduced water and nitrogen use) to keep the plants healthy. Additionally, the Rubus plants comprising a mutation in a gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) and having compact growth provide an increase in fruit quality (increased Brix, increased Brix:acidity ratio, increased flavor score) and an increase in marketable fruit produced.

Yield can be defined as the measurable produce of economic value from a crop. Yield can be defined in the scope of quantity and/or quality. Yield can be directly dependent on several factors, for example, the number and size of organs, plant architecture (such as the number of branches, plant biomass, e.g., increased root biomass, steeper root angle and/or longer roots, and the like), flowering time and duration, grain fill period. Root architecture and development, photosynthetic efficiency, nutrient uptake, stress tolerance, early vigor, delayed senescence and functional stay green phenotypes may be factors in determining yield. Optimizing the above-mentioned factors can therefore contribute to increasing crop yield.

Reference herein to an increase/improvement in yield-related traits can also be taken to mean an increase in biomass (weight) of one or more parts of a plant, which can include above ground and/or below ground (harvestable) plant parts. In particular, such harvestable parts are fruits, and performance of the methods of the disclosure results in plants with increased yield and in particular increased fruit yield per acre relative to the fruit yield of suitable control plants.

Increased yield of a plant of the present disclosure can be measured in a number of ways, including test weight, fruit number per plant, fruit weight, fruit number per unit area (for example, fruits, or weight of fruits, per acre), bushels per acre, tons per acre, or kilo per hectare.

“Increased yield” can manifest as one or more of the following: (i) increased plant biomass (weight) of one or more parts of a plant, particularly aboveground (harvestable) parts, of a plant, increased root biomass (increased number of roots, increased root thickness, increased root length) or increased biomass of any other harvestable part; or (ii) increased early vigor, defined herein as an improved seedling aboveground area approximately three weeks post-germination.

Increased “harvesting efficiency” as used herein refers to the amount of harvested fruit (e.g., pounds of fruit) picked per person per minute of time spent picking (harvesting) the fruit.

As used herein, “unmarketable fruit” refers to fruit that do not meet quality standards for fresh retail sale. Factors that may result in unmarketable fruit include for example, small size, damage due abiotic stress, pests or disease, or conditions, if the fruit is misshapen and/or discolored, or poor flavor.

As used herein, “marketable fruit” refers to fruit that meets quality standards for fresh retail sale for Rubus berries that include, but are not limited to, size, appearance (color, free from decay or damage) and quality (flavor, ripeness, color).

“Brix” is a standard method for measuring the percentage of dissolved solids, primarily sugars, in a liquid solution. Brix is provided as ° Bx, and is typically measured using a refractometer. 1 degree Brix)=1 gram sucrose/100 grams of solution. Brix may be used as an indicator of fruit quality.

As used herein, “acidity” is a commonly used indicator of fruit quality and is the total amount of acid present in the fruit. This is typically measured as titratable acidity (TA) w/w as citric acid.

The “Brix:acidity ratio” is another indicator of fruit quality determined by dividing the Brix by the titratable acidity.

As used herein, “flavor score” refers to a scale of 1-9 that is based on a complex evaluation of fruit, including the flavor of the fruit (tartness/sweetness), its aroma and other factors. Flavor score may be based on consumer evaluation.

As used herein, “an improved agronomic performance” refers to any qualitative or quantitative parameter used to measure the performance of a crop such as productivity (yield), health of the plant, quality of the harvested product, and resource use efficiency. An improved agronomic performance may be an increase in yield, increase in the quality and amount of marketable product (e.g., fruit), decreased water and nitrogen use per plant or amount marketable product and the like.

As used herein, the terms “nucleic acid,” “nucleic acid molecule,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “nucleic acid construct,” “oligonucleotide” and “polynucleotide” are also used interchangeably herein to refer to a heteropolymer of nucleotides. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25. A “5′ region” as used herein can mean the region of a polynucleotide that is nearest the 5′ end of the polynucleotide. Thus, for example, an element in the 5′ region of a polynucleotide can be located anywhere from the first nucleotide located at the 5′ end of the polynucleotide to the nucleotide located halfway through the polynucleotide. A “3′ region” as used herein can mean the region of a polynucleotide that is nearest the 3′ end of the polynucleotide. Thus, for example, an element in the 3′ region of a polynucleotide can be located anywhere from the first nucleotide located at the 3′ end of the polynucleotide to the nucleotide located halfway through the polynucleotide.

As used herein with respect to nucleic acids, the term “fragment” or “portion” refers to a nucleic acid that is reduced in length relative (e.g., reduced by 1, 2, 3, 4, 5, 6, 7, 8, 9, 10, 11, 12, 13, 14, 15, 16, 17, 18, 19, 20, 20, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, or 900 or more nucleotides or any range or value therein) to a reference nucleic acid and that comprises, consists essentially of and/or consists of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. As an example, a repeat sequence of guide nucleic acid of this invention may comprise a “portion” of a wild type CRISPR-Cas repeat sequence (e.g., a wild Type CRISPR-Cas repeat; e.g., a repeat from the CRISPR Cas system of, for example, a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or a Cas14c, and the like).

In some embodiments, a nucleic acid fragment may comprise, consist essentially of or consist of about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, 550, 600, 650, 700, 750, 800, 850, or 900 or more consecutive nucleotides or any range or value therein of a nucleic acid encoding a TFL protein, optionally a TFL fragment may be about 50 nucleotides to about 300 nucleotides in length, about 50 nucleotides to about 350 nucleotides in length, about 50 nucleotides to about 400 nucleotides in length, about 50 nucleotides to about 450 nucleotides in length, about 50 nucleotides to about 500 nucleotides in length, about 50 nucleotides to about 600 nucleotides in length, about 50 nucleotides to about 800 nucleotides in length, about 50 nucleotides to about 900 nucleotides in length, about 50 nucleotides to about 950 nucleotides in length, about 100 nucleotides to about 300 nucleotides in length, about 100 nucleotides to about 350 nucleotides in length, about 100 nucleotides to about 400 nucleotides in length, about 100 nucleotides to about 450 nucleotides in length, about 100 nucleotides to about 500 nucleotides in length, about 100 nucleotides to about 600 nucleotides in length, about 100 nucleotides to about 800 nucleotides in length, about 100 nucleotides to about 900 nucleotides in length, or about 100 nucleotides to about 950 nucleotides in length, or any range or value therein. As an example, a fragment or portion may be a fragment or portion of any one of SEQ ID NOs:72, 73, 106, 107, 114, 115, 126, 127, 140, 141, 153, 162, 163, 172, 173, 184, 185, 214, 215, 234, 235, or 252 (e.g., SEQ ID NOs:75-105, 109-113, 117-125, 129-139, 143-152, 155-161, 165-171, 175-183, 187-192, 217-233, 237-246, or 254-270.

In some embodiments, a “portion” in reference to a nucleic acid means at least 2, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 135, 136, 137, 138, 139, 140, 141, 142, 143, 144, 145, 150, 160, 170, 171, 172, 173, 174, 175, 176, 177, 178, 179, 180, 190, 200, 210, 215, 216, 217, 218, 219, 220, 221, 222, 223, 224, 225, 230, 240, 250, 260, 270, 280, 290, 291, 292, 293, 294, 295,296, 297, 298, 299, or 300 or more consecutive nucleotides from a gene (e.g., consecutive nucleotides from a TFL gene). As an example, a fragment or portion may be a fragment or portion of any one of SEQ ID NOs:72, 73, 106, 107, 114, 115, 126, 127, 140, 141, 153, 162, 163, 172, 173, 184, 185, 214, 215, 234, 235, or 252 (e.g., SEQ ID NOs:75-105, 109-113, 117-125, 129-139, 143-152, 155-161, 165-171, 175-183, 187-192, 217-233, 237-246, or 254-270).

In some embodiments, a nucleic acid fragment of a TFL gene may be the result of a deletion of nucleotides from the 3′ end, the 5′ end, and/or from within any region (e.g., within an exon, a coding region) of a gene encoding a TFL protein. In some embodiments, a deletion of a portion of a gene encoding a TFL protein may comprise a deletion of a portion of consecutive nucleotides from the 5′ end, the 3′ end, or from within any region of a gene, for example, a deletion of a portion of consecutive nucleotides from the 5′ end, the 3′ end, or from within any region of any one of the nucleotide sequences of SEQ ID NOs:72, 73, 106, 107, 114, 115, 126, 127, 140, 141, 153, 162, 163, 172, 173, 184, 185, 214, 215, 234, 235, or 252. In some embodiments, a deletion of a portion of a TFL gene may comprise deletion of a portion of consecutive nucleotides from any one of the nucleotide sequences of SEQ ID NOs:72, 73, 106, 107, 114, 115, 126, 127, 140, 141, 153, 162, 163, 172, 173, 184, 185, 214, 215, 234, 235, or 252.

In some embodiments, a deletion of a portion of a TFL gene may comprise a deletion of a portion of consecutive nucleotides from any one of the nucleotide sequences of SEQ ID NOs:72, 106, 114, 126, 140, 162, 172, 184, 214, 234, or 252 from about 3 consecutive nucleotides to about 2600 consecutive nucleotides or more (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175,200, 225, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500 or 2600 or more consecutive nucleotides, or any range or value therein).

In some embodiments, a deletion of a TFL gene may comprise a deletion of about 3 to about 515 or more nucleotides of SEQ ID NOs:73, 107, 115, 127, 141, 153, 163, 173, or 185, 215, or 235 (e.g., about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 consecutive nucleotides to about 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 350, 455, 460, 465, 470, 475, 480, 485, 490, 495, 500, 505, 510, 515, 516, 517, 518, 519, 520 or more consecutive nucleotides or any range or value therein, optionally about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 to about 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 consecutive nucleotides).

In some embodiments, a deletion in a TFL gene as described herein, or a deletion in another endogenous gene of interest as described herein (e.g., CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof), may be a null allele, which when comprised in a Rubus plant can result in the plant exhibiting a phenotype of reduced time to flowering in the plant, a longer duration of flowering, one or more improved yield characteristics and/or a more compact growth habit (e.g., reduced height, reduced/shortened stem length, reduced/shortened internode length, reduced cane length, reduced lateral branching, increased determinacy as compared to a Rubus plant not comprising the same deletion. In some embodiments, such a deletion may be a dominant-negative allele, semi-dominant allele, weak loss of function allele, a null allele, or a hypomorphic mutation, which when comprised in a plant can result in the plant exhibiting a phenotype of reduced time to flowering in the plant, a longer duration of flowering, one or more improved yield characteristics (e.g., increased fruit production) and/or a more compact growth habit as described herein.

As used herein, a “reduced height” of a Rubus plant comprising a mutation in an endogenous gene as described herein (e.g., TFL) may be a reduction in height of about 10% to about 70% (e.g., a decrease in height of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70%, optionally an decrease in height of about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65%) or any range or value therein, as compared to a control Rubus plant. In some embodiments, reduction in height may be an average decrease of at least about 35, 36, 37, 38, 39, 40, 41, 42, 43, 44, 45, 46, 47, 48, 49, or 50% as compared to a control Rubus plant.

As used herein, a “reduced cane length” of a Rubus plant comprising a mutation in an endogenous gene as described herein (e.g., TFL) may be a reduction in cane length of about 10% to about 70% (e.g., a decrease in cane length of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70%, optionally an decrease in cane length of about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65%) or any range or value therein, as compared to a control Rubus plant. In some embodiments, reduction in cane length may be an average decrease of at least about 35, 36, 37, 38, 39, 40, 41, 42, 43, 44, 45, 46, 47, 48, 49, or 50% as compared to a control Rubus plant.

As used herein, an “increase in cane number” of a Rubus plant comprising a mutation in an endogenous gene as described herein (e.g., TFL) may be an increase in cane length of about 10% to about 160% (e.g., an increase of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, or 160%, optionally an increase of about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, or 150%) or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%) or any range or value therein, as compared to a control Rubus plant. In some embodiments, an increase in cane number may be an average increase of at least about 50, 51, 52, 53, 54, 55, 56, 57, 58, 59, 60, 61, 62, 63, 64, 65, 66, 67, 68, 69, or 70% as compared to a control Rubus plant.

In some embodiments, a “sequence-specific nucleic acid binding domain” may bind to one or more fragments or portions of nucleotide sequences (e.g., DNA, RNA) encoding, for example, TFL polypeptides as described herein.

As used herein with respect to polypeptides, the term “fragment” or “portion” may refer to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400 or more consecutive amino acids of a reference polypeptide. In some embodiments, a polypeptide fragment may comprise, consist essentially of or consist of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, or 172 or more consecutive amino acid residues (or any range or value therein) of a TFL1 polypeptide (e.g., a fragment or a portion of any one of SEQ ID NOs:74, 108, 116, 128, 142, 154, 164, 174, 186, 216, 236 or 253 (e.g., SEQ ID NOs:193-197, 247, 248, or 271-276).

In some embodiments, a “portion” may be related to the number of amino acids that are deleted from a polypeptide. Thus, for example, a deleted “portion” of an TFL polypeptide may comprise at least one amino acid residue (e.g., at least 1, or at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, or 172, or more consecutive amino acid residues) deleted from any one of the amino acid sequences SEQ ID NOs:74, 108, 116, 128, 142, 154, 164, 174, 186, 216, 236 or 253 (e.g., SEQ ID NOs:193-197, 247, 248, or 271-276).

In some embodiments, a deletion of a portion of a TFL protein may comprise a deletion of a portion of consecutive amino acid residues from the N- or C-terminus of or within any one of the amino acid sequences of SEQ ID NOs:74, 108, 116, 128, 142, 154, 164, 174, 186, 216, 236 or 253 (e.g., SEQ ID NOs:193-197, 247, 248, or 271-276). Thus, in some embodiments, a fragment or portion of a TFL polypeptide that is deleted may be within a TFL polypeptide from amino acid residue 20, 21, 22, 23, 24, 25, 30, 35, 40, 45, 50, 60, 70, 71, 72, 73, or 74 to amino acid residue 115, 116, 117, 118, 119, or 120 with reference amino acid position numbering of SEQ ID NOs:74, 108, 116, 128, 142, 154, 164, or 174, or from amino acid residue 60, 70, 71, 72, 73, 74, 75, 76, 78, 79, 80 or to amino acid residue 117, 118, 119, 120, 121, 122, 123, 124, or 125 with reference amino acid position numbering of SEQ ID NO:186, 216, 236 or 253.

In some embodiments, a deletion of a portion of a TFL polypeptide may comprise a deletion of a portion of consecutive amino acid residues from the C-terminus or N-terminus of any one of the amino acid sequences of SEQ ID NOs:74, 108, 116, 128, 142, 154, 164, 174, 186, 216, 236 or 253. In some embodiments, a deletion of a portion of a TFL polypeptide may comprise a deletion of a portion of consecutive amino acid residues from the C-terminus or N-terminus of any one of the amino acid sequences SEQ ID NOs:74, 108, 116, 128, 142, 154, 164, 174, 186, 216, 236 or 253 of from about 1, 2, 3, 4, 5, 6, 7, 8, 9, or 10 consecutive amino acids to about 50, about 75, about 100, about 120, about 130, about 150, about 160, or about 172 consecutive amino acids, or about 5, 6, 7, 8, 9, 10, 11, 12, 13, 14, 15, 16, 17, 18, 19, 20, 21, 22, 23, 24, 25, 26, 27, 28, 29, or 30 consecutive amino acids to about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, or 170 or more consecutive amino acids (or any range or value therein). In some embodiments, such a deletion may be a null allele, which when comprised in a Rubus plant (e.g., a blackberry plant, a red raspberry plant, a black raspberry plant, etc.) may result in plant having a compact growth habit as compared to a Rubus plant devoid of the deletion. In some embodiments, such a deletion may be a dominant-negative allele, semi-dominant allele, weak loss of function allele, a null allele, or a hypomorphic mutation, which when comprised in a Rubus plant may result in the plant having a compact growth habit as compared to a Rubus plant devoid of the deletion.

As used herein with respect to nucleic acids, the term “functional fragment” refers to nucleic acid that encodes a functional fragment of a polypeptide. A “functional fragment” with respect to a polypeptide is a fragment of a polypeptide that retains one or more of the activities of the native reference polypeptide.

The term “gene,” as used herein, refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The term “mutation” refers to point mutations (e.g., missense, or nonsense, or insertions or deletions of single base pairs that result in frame shifts), insertions, deletions, inversions and/or truncations. When the mutation is a substitution of a residue within an amino acid sequence with another residue, or a deletion or insertion of one or more residues within a sequence, the mutations are typically described by identifying the original residue followed by the position of the residue within the sequence and by the identity of the newly substituted residue. A truncation can include a truncation at the C-terminal end of a polypeptide or at the N-terminal end of a polypeptide. A truncation of a polypeptide can be the result of a deletion of the corresponding 5′ end or 3′ end of the gene encoding the polypeptide. A frameshift mutation can occur when deletions or insertions of one or more base pairs are introduced into a gene, optionally resulting in an out-of-frame mutation or an in-frame mutation. Frameshift mutations in a gene can result in the production of a polypeptide that is longer, shorter or the same length as the wild type polypeptide depending on when the first stop codon occurs following the mutated region of the gene. As an example, an out-of-frame mutation that produces a premature stop codon can produce a polypeptide that is shorter that the wild type polypeptide, or, in some embodiments, the polypeptide may be absent/undetectable. In some embodiments, a mutation may be a DNA inversion, optionally a DNA inversion having a length of about 10 to about 2000 consecutive base pairs.

The terms “complementary” or “complementarity,” as used herein, refer to the natural binding of polynucleotides under permissive salt and temperature conditions by base-pairing. For example, the sequence “A-G-T” (5′ to 3′) binds to the complementary sequence “T-C-A” (3′ to 5′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

“Complement,” as used herein, can mean 100% complementarity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity) to the comparator nucleotide sequence.

Different nucleic acids or proteins having homology are referred to herein as “homologues.” The term homologue includes homologous sequences from the same and from other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to said nucleotide sequence of the invention.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences, or polypeptide sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, about 100 nucleotides to about 200 nucleotides, about 100 nucleotides to about 300 nucleotides, about 100 nucleotides to about 400 nucleotides, about 100 nucleotides to about 500 nucleotides, about 100 nucleotides to about 600 nucleotides, about 100 nucleotides to about 800 nucleotides, about 100 nucleotides to about 900 nucleotides, or more in length, or any range therein, up to the full length of the sequence. In some embodiments, nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40, 50, 60, 70 or 80 nucleotides or more).

In some embodiments of the invention, the substantial identity exists over a region of consecutive amino acid residues of a polypeptide of the invention that is about 3 amino acid residues to about 20 amino acid residues, about 5 amino acid residues to about 25 amino acid residues, about 7 amino acid residues to about 30 amino acid residues, about 10 amino acid residues to about 25 amino acid residues, about 15 amino acid residues to about 30 amino acid residues, about 20 amino acid residues to about 40 amino acid residues, about 25 amino acid residues to about 40 amino acid residues, about 25 amino acid residues to about 50 amino acid residues, about 30 amino acid residues to about 50 amino acid residues, about 40 amino acid residues to about 50 amino acid residues, about 40 amino acid residues to about 70 amino acid residues, about 50 amino acid residues to about 70 amino acid residues, about 60 amino acid residues to about 80 amino acid residues, about 70 amino acid residues to about 80 amino acid residues, about 90 amino acid residues to about 100 amino acid residues, or more amino acid residues in length, and any range therein, up to the full length of the sequence. In some embodiments, polypeptide sequences can be substantially identical to one another over at least about 8 consecutive amino acid residues (e.g., about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350 or more amino acids in length or more consecutive amino acid residues). In some embodiments, two or more TFL polypeptides may be identical or substantially identical (e.g., at least 70% to 99.9% identical, e.g., about 70%, 71%, 72%, 73%, 74%, 75%, 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, 99.5%. 99.9% identical or any range or value therein) to one another over at least about 44 consecutive amino acid residues (e.g., SEQ ID NOs:193-197, 247, 248, or 271-276). In some embodiments, two or more TFL proteins may be substantially identical across consecutive amino acid residues 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 145, 150, 155, 160, 165, 170, 171, 172 or more of any one of the amino acid sequences of SEQ ID NOs:74, 108, 116, 128, 142, 154, 164, 174, 186, 216, 236 or 253.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Two nucleotide sequences may also be considered substantially complementary when the two sequences hybridize to each other under stringent conditions. In some embodiments, two nucleotide sequences considered to be substantially complementary hybridize to each other under highly stringent conditions.

A polynucleotide and/or recombinant nucleic acid construct of this invention (e.g., expression cassettes and/or vectors) may be optimized for expression. In some embodiments, the polynucleotides, nucleic acid constructs, expression cassettes, and/or vectors of the editing systems of the invention (e.g., comprising/encoding a sequence-specific DNA binding domain (e.g., a sequence-specific DNA binding domain from a polynucleotide-guided endonuclease, a zinc finger nuclease, a transcription activator-like effector nuclease (TALEN), an Argonaute protein, and/or a CRISPR-Cas endonuclease (e.g., CRISPR-Cas effector protein) (e.g., a Type I CRISPR-Cas effector protein, a Type II CRISPR-Cas effector protein, a Type III CRISPR-Cas effector protein, a Type IV CRISPR-Cas effector protein, a Type V CRISPR-Cas effector protein or a Type VI CRISPR-Cas effector protein)), a nuclease (e.g., an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN)), deaminase proteins/domains (e.g., adenine deaminase, cytosine deaminase), a polynucleotide encoding a reverse transcriptase protein or domain, a polynucleotide encoding a 5′-3′ exonuclease polypeptide, and/or affinity polypeptides, peptide tags, etc.) may be optimized for expression in a plant. In some embodiments, the optimized nucleic acids, polynucleotides, expression cassettes, and/or vectors of the invention have about 70% to about 99.9% (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%. 99.9% or 100%) identity or more to the reference nucleic acids, polynucleotides, expression cassettes, and/or vectors that have not been so optimized.

A polynucleotide or nucleic acid construct of the invention may be operatively associated with a variety of promoters and/or other regulatory elements for expression in a plant and/or a cell of a plant. Thus, in some embodiments, a polynucleotide or nucleic acid construct of this invention may further comprise one or more promoters, introns, enhancers, and/or terminators operably linked to one or more nucleotide sequences. In some embodiments, a promoter may be operably associated with an intron (e.g., Ubi1 promoter and intron). In some embodiments, a promoter associated with an intron maybe referred to as a “promoter region” (e.g., Ubi1 promoter and intron).

By “operably linked” or “operably associated” as used herein in reference to polynucleotides, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, nucleic acid sequences can be present between a promoter and the nucleotide sequence, and the promoter can still be considered “operably linked” to the nucleotide sequence.

As used herein, the term “linked,” in reference to polypeptides, refers to the attachment of one polypeptide to another. A polypeptide may be linked to another polypeptide (at the N-terminus or the C-terminus) directly (e.g., via a peptide bond) or through a linker.

The term “linker” is art-recognized and refers to a chemical group, or a molecule linking two molecules or moieties, e.g., two domains of a fusion protein, such as, for example, a DNA binding polypeptide or domain and peptide tag and/or a reverse transcriptase and an affinity polypeptide that binds to the peptide tag; or a DNA endonuclease polypeptide or domain and peptide tag and/or a reverse transcriptase and an affinity polypeptide that binds to the peptide tag. A linker may be comprised of a single linking molecule or may comprise more than one linking molecule. In some embodiments, the linker can be an organic molecule, group, polymer, or chemical moiety such as a bivalent organic moiety. In some embodiments, the linker may be an amino acid or it may be a peptide. In some embodiments, the linker is a peptide.

In some embodiments, a peptide linker useful with this invention may be about 2 to about 100 or more amino acids in length. In some embodiments, a peptide linker may be a GS linker.

As used herein, the term “linked,” or “fused” in reference to polynucleotides, refers to the attachment of one polynucleotide to another. In some embodiments, two or more polynucleotide molecules may be linked by a linker that can be an organic molecule, group, polymer, or chemical moiety such as a bivalent organic moiety. A polynucleotide may be linked or fused to another polynucleotide (at the 5′ end or the 3′ end) via a covalent or non-covenant linkage or binding, including e.g., Watson-Crick base-pairing, or through one or more linking nucleotides. In some embodiments, a polynucleotide motif of a certain structure may be inserted within another polynucleotide sequence (e.g. extension of the hairpin structure in the guide RNA). In some embodiments, the linking nucleotides may be naturally occurring nucleotides. In some embodiments, the linking nucleotides may be non-naturally occurring nucleotides.

A “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1) (See, Walker et al. Plant Cell Rep. 23:727-735 (2005); Li et al. Gene 403:132-142 (2007); Li et al. Mol Biol. Rep. 37:1143-1154 (2010)). PrbcS1 and Pactin are constitutive promoters and Pnr and Pdca1 are inducible promoters. Pnr is induced by nitrate and repressed by ammonium (Li et al. Gene 403:132-142 (2007)) and Pdca1 is induced by salt (Li et al. Mol Biol. Rep. 37:1143-1154 (2010)). In some embodiments, a promoter useful with this invention is RNA polymerase II (Pol II) promoter. In some embodiments, a U6 promoter or a 7SL promoter from Zea mays may be useful with constructs of this invention. In some embodiments, the U6c promoter and/or 7SL promoter from Zea mays may be useful for driving expression of a guide nucleic acid. In some embodiments, a U6c promoter, U6i promoter and/or 7SL promoter from Glycine max may be useful with constructs of this invention. In some embodiments, the U6c promoter, U6i promoter and/or 7SL promoter from Glycine max may be useful for driving expression of a guide nucleic acid.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al., 1991. Plant Science 79: 87-94), maize (Christensen et al., 1989. Plant Molec. Biol. 12: 619-632), and arabidopsis (Norris et al. 1993. Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. (Mol. Gen. Genet. 231: 150-160 (1991)) can be easily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula, Plant Molec. Biol. 12:579-589 (1989)). Non-limiting examples of tissue-specific promoters include those associated with genes encoding the seed storage proteins (such as 0-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Tissue-specific or tissue-preferential promoters useful for the expression of the nucleotide sequences of the invention in plants, particularly maize, include but are not limited to those that direct expression in root, pith, leaf or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond (FEBS 290:103-106 (1991); EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. Plant Biotechnol. Reports 9(5):297-306 (2015)), ZmSTK2_USP from maize (Wang et al. Genome 60(6):485-495 (2017)), LAT52 and LAT59 from tomato (Twell et al. Development 109(3):705-713 (1990)), Zm13 (U.S. Pat. No. 10,421,972), PLA₂-δ promoter from arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. The Plant Cell 18:2958-2970 (2006)), the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAM S) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genet. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genet. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubi1 promoter and intron (see, e.g., SEQ ID NO:21 and SEQ ID NO:22).

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

In some embodiments, a polynucleotide and/or a nucleic acid construct of the invention can be an “expression cassette” or can be comprised within an expression cassette. As used herein, “expression cassette” means a recombinant nucleic acid molecule comprising, for example, a one or more polynucleotides of the invention (e.g., a polynucleotide encoding a sequence-specific DNA binding domain, a polynucleotide encoding a deaminase protein or domain, a polynucleotide encoding a reverse transcriptase protein or domain, a polynucleotide encoding a 5′-3′ exonuclease polypeptide or domain, a guide nucleic acid and/or reverse transcriptase (RT) template), wherein polynucleotide(s) is/are operably associated with one or more control sequences (e.g., a promoter, terminator and the like). Thus, in some embodiments, one or more expression cassettes may be provided, which are designed to express, for example, a nucleic acid construct of the invention (e.g., a polynucleotide encoding a sequence-specific DNA binding domain, a polynucleotide encoding a nuclease polypeptide/domain, a polynucleotide encoding a deaminase protein/domain, a polynucleotide encoding a reverse transcriptase protein/domain, a polynucleotide encoding a 5′-3′ exonuclease polypeptide/domain, a polynucleotide encoding a peptide tag, and/or a polynucleotide encoding an affinity polypeptide, and the like, or comprising a guide nucleic acid, an extended guide nucleic acid, and/or RT template, and the like). When an expression cassette of the present invention comprises more than one polynucleotide, the polynucleotides may be operably linked to a single promoter that drives expression of all of the polynucleotides or the polynucleotides may be operably linked to one or more separate promoters (e.g., three polynucleotides may be driven by one, two or three promoters in any combination). When two or more separate promoters are used, the promoters may be the same promoter, or they may be different promoters. Thus, a polynucleotide encoding a sequence specific DNA binding domain, a polynucleotide encoding a nuclease protein/domain, a polynucleotide encoding a CRISPR-Cas effector protein/domain, a polynucleotide encoding an deaminase protein/domain, a polynucleotide encoding a reverse transcriptase polypeptide/domain (e.g., RNA-dependent DNA polymerase), and/or a polynucleotide encoding a 5′-3′ exonuclease polypeptide/domain, a guide nucleic acid, an extended guide nucleic acid and/or RT template when comprised in a single expression cassette may each be operably linked to a single promoter, or separate promoters in any combination.

An expression cassette comprising a nucleic acid construct of the invention may be chimeric, meaning that at least one of its components is heterologous with respect to at least one of its other components (e.g., a promoter from the host organism operably linked to a polynucleotide of interest to be expressed in the host organism, wherein the polynucleotide of interest is from a different organism than the host or is not normally found in association with that promoter). An expression cassette may also be one that is naturally occurring but has been obtained in a recombinant form useful for heterologous expression.

An expression cassette can optionally include a transcriptional and/or translational termination region (i.e., termination region) and/or an enhancer region that is functional in the selected host cell. A variety of transcriptional terminators and enhancers are known in the art and are available for use in expression cassettes. Transcriptional terminators are responsible for the termination of transcription and correct mRNA polyadenylation. A termination region and/or the enhancer region may be native to the transcriptional initiation region, may be native to, for example, a gene encoding a sequence-specific DNA binding protein, a gene encoding a nuclease, a gene encoding a reverse transcriptase, a gene encoding a deaminase, and the like, or may be native to a host cell, or may be native to another source (e.g., foreign or heterologous to, for example, to a promoter, to a gene encoding a sequence-specific DNA binding protein, a gene encoding a nuclease, a gene encoding a reverse transcriptase, a gene encoding a deaminase, and the like, or to the host cell, or any combination thereof).

An expression cassette of the invention also can include a polynucleotide encoding a selectable marker, which can be used to select a transformed host cell. As used herein, “selectable marker” means a polynucleotide sequence that when expressed imparts a distinct phenotype to the host cell expressing the marker and thus allows such transformed cells to be distinguished from those that do not have the marker. Such a polynucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acid molecules/constructs and polynucleotide sequences described herein can be used in connection with vectors. The term “vector” refers to a composition for transferring, delivering or introducing a nucleic acid (or nucleic acids) into a cell. A vector comprises a nucleic acid construct (e.g., expression cassette(s)) comprising the nucleotide sequence(s) to be transferred, delivered or introduced. Vectors for use in transformation of host organisms are well known in the art. Non-limiting examples of general classes of vectors include viral vectors, plasmid vectors, phage vectors, phagemid vectors, cosmid vectors, fosmid vectors, bacteriophages, artificial chromosomes, minicircles, or Agrobacterium binary vectors in double or single stranded linear or circular form which may or may not be self-transmissible or mobilizable. In some embodiments, a viral vector can include, but is not limited, to a retroviral, lentiviral, adenoviral, adeno-associated, or herpes simplex viral vector. A vector as defined herein can transform a prokaryotic or eukaryotic host either by integration into the cellular genome or exist extrachromosomally (e.g., autonomous replicating plasmid with an origin of replication). Additionally included are shuttle vectors by which is meant a DNA vehicle capable, naturally or by design, of replication in two different host organisms, which may be selected from actinomycetes and related species, bacteria and eukaryotic (e.g., higher plant, mammalian, yeast, or fungal cells). In some embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter and/or other regulatory elements and in the case of cDNA this may be under the control of an appropriate promoter and/or other regulatory elements for expression in the host cell. Accordingly, a nucleic acid or polynucleotide of this invention and/or expression cassettes comprising the same may be comprised in vectors as described herein and as known in the art.

As used herein, “contact,” “contacting,” “contacted,” and grammatical variations thereof, refer to placing the components of a desired reaction together under conditions suitable for carrying out the desired reaction (e.g., transformation, transcriptional control, genome editing, nicking, and/or cleavage). As an example, a target nucleic acid may be contacted with a sequence-specific nucleic acid binding protein (e.g., polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein)) and a deaminase or a nucleic acid construct encoding the same, under conditions whereby the sequence-specific DNA binding protein, the reverse transcriptase and/or the deaminase are expressed and the sequence-specific DNA binding protein binds to the target nucleic acid, and the reverse transcriptase and/or deaminase may be fused to either the sequence-specific DNA binding protein or recruited to the sequence-specific DNA binding protein (via, for example, a peptide tag fused to the sequence-specific DNA binding protein and an affinity tag fused to the reverse transcriptase and/or deaminase) and thus, the deaminase and/or reverse transcriptase is positioned in the vicinity of the target nucleic acid, thereby modifying the target nucleic acid. Other methods for recruiting reverse transcriptase and/or deaminase may be used that take advantage of other protein-protein interactions, and also RNA-protein interactions and chemical interactions may be used for protein-protein and protein-nucleic acid recruitment.

As used herein, “modifying” or “modification” in reference to a target nucleic acid includes editing (e.g., mutating), covalent modification, exchanging/substituting nucleic acids/nucleotide bases, deleting, cleaving, nicking, and/or altering transcriptional control of a target nucleic acid. In some embodiments, a modification may include one or more single base changes (SNPs) of any type.

“Introducing,” “introduce,” “introduced” (and grammatical variations thereof) in the context of a polynucleotide of interest means presenting a nucleotide sequence of interest (e.g., polynucleotide, RT template, a nucleic acid construct, and/or a guide nucleic acid) to a plant, plant part thereof, or cell thereof, in such a manner that the nucleotide sequence gains access to the interior of a cell.

The terms “transformation” or transfection” may be used interchangeably and as used herein refer to the introduction of a heterologous nucleic acid into a cell. Transformation of a cell may be stable or transient. Thus, in some embodiments, a host cell or host organism (e.g., a plant) may be stably transformed with a polynucleotide/nucleic acid molecule of the invention. In some embodiments, a host cell or host organism may be transiently transformed with a polynucleotide/nucleic acid molecule of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. “Genome” as used herein includes the nuclear and the plastid genome, and therefore includes integration of the nucleic acid into, for example, the chloroplast or mitochondrial genome. Stable transformation as used herein can also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a host organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Accordingly, in some embodiments, nucleotide sequences, polynucleotides, nucleic acid constructs, and/or expression cassettes of the invention may be expressed transiently and/or they can be stably incorporated into the genome of the host organism. Thus, in some embodiments, a nucleic acid construct of the invention (e.g., one or more expression cassettes comprising polynucleotides for editing as described herein) may be transiently introduced into a cell with a guide nucleic acid and as such, no DNA is maintained in the cell.

A nucleic acid construct of the invention may be introduced into a plant cell by any method known to those of skill in the art. Non-limiting examples of transformation methods include transformation via bacterial-mediated nucleic acid delivery (e.g., via Agrobacteria), viral-mediated nucleic acid delivery, silicon carbide or nucleic acid whisker-mediated nucleic acid delivery, liposome mediated nucleic acid delivery, microinjection, microparticle bombardment, calcium-phosphate-mediated transformation, cyclodextrin-mediated transformation, electroporation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

In some embodiments of the invention, transformation of a cell may comprise nuclear transformation. In other embodiments, transformation of a cell may comprise plastid transformation (e.g., chloroplast transformation). In still further embodiments, nucleic acids of the invention may be introduced into a cell via conventional breeding techniques. In some embodiments, one or more of the polynucleotides, expression cassettes and/or vectors may be introduced into a plant cell via Agrobacterium transformation.

A polynucleotide therefore can be introduced into a plant, plant part, plant cell in any number of ways that are well known in the art. The methods of the invention do not depend on a particular method for introducing one or more nucleotide sequences into a plant, only that they gain access to the interior the cell. Where more than polynucleotide is to be introduced, they can be assembled as part of a single nucleic acid construct, or as separate nucleic acid constructs, and can be located on the same or different nucleic acid constructs. Accordingly, the polynucleotide can be introduced into the cell of interest in a single transformation event, or in separate transformation events, or, alternatively, a polynucleotide can be incorporated into a plant as part of a breeding protocol.

Plant flowering and architecture are mediated through a small group of regulatory factors with structural similarities to Phosphatidyl Ethanolamine Binding Proteins (PEBP). A naturally occurring variant in TFL1 in wild strawberry, for example, reduces flowering time (Plant Journal 69, 116-12 (2012)). Individual TFL-like family members can be grouped into TFL1, CEN and BFT clades based on sequence identity and synteny. The dominant function of TFL1, CEN and BFT members is to regulate flowering time. However, there are distinct secondary functions associated with individual members that can be exploited for crop improvement. Endogenous genes, in addition to TFL, CEN and BFT, may also be mutated to confer advantageous flowering and architectural traits on a Rubus plant such as BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, or orthologues thereof, or orthologues thereof.

Accordingly, the present invention is directed to methods for improving at least one agronomic trait in a Rubus plant, the method comprising planting Rubus plants at a high density, wherein the Rubus plants comprise at least one mutation in an endogenous gene including, but not limited to, TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX IPT, PIF4/PIF5, PHYB, HFR1, BFT, or orthologues thereof, and exhibit compact growth as compared to a control Rubus plant, which does not comprise the at least one mutation, compact growth and cannot be planted at a high density without compromising at least one agronomic trait such as yield, fruit quality, resource use efficiency and/or harvesting efficiency. In some embodiments, the invention is directed to methods for improving at least one agronomic trait in a Rubus plant, the method comprising planting Rubus plants at a high density, wherein the Rubus plants comprise at least one mutation in an endogenous TERMINAL FLOWER (TFL) gene (e.g., one or more TFL genes) encoding a TFL polypeptide and exhibiting compact growth as compared to a control Rubus plant.

In some embodiments, a method of cultivating Rubus plants to improve agronomic performance is provided, comprising planting Rubus plants at a high density, wherein the Rubus plants comprise at least one mutation in at least one endogenous gene associated with plant architecture and exhibit a compact growth habit resulting in an increased in yield per acre and improved agronomic efficiency as compared to control Rubus plants. In some embodiments, a method of improving at least one agronomic trait in Rubus plants is provided, comprising planting Rubus plants at a high density, wherein the Rubus plants comprise at least one mutation in one or more endogenous genes that confer modifications in plant architecture and exhibit a compact growth habit resulting in improvement of at least one agronomic trait as compared to a control Rubus plant (devoid of the at least one mutation in the one or more endogenous genes and does not exhibit compact growth). In some embodiments, the at least one endogenous gene associated with plant architecture is TFL (TERMINAL FLOWER), CEN (CENTRORADIALIS), BRC1 (BRANCHED1), AP1 (APETALA1), FT (FLOWERING LOCUS T), GA20ox/GA3ox (GIBBERELLIN 20-OXIDASE, GIBBERELLIN 3-OXIDASE), GA2ox (GIBBERELLIN 2-OXIDASE), GID1 (GIBBERELLIN INSENSITIVE DWARF1), DELLA (DELLA PROTEIN (GA SIGNALING REPRESSOR), SLY1/GID2 (SLEEPY1, GIBBERELLIN INSENSITIVE DWARF2), YUCCA (YUCCA FLAVIN MONOOXYGENASE-LIKE), PIN1/PIN3/PIN4 (PIN-FORMED 1 AUXIN TRANSPORTER, PIN3, PIN4), AUX1, BR1/BR2 (BR1, BR2), AUX/IAA (AUX, IAA), DWF4/CPD (DWARF4 STEROL C-22 HYDROXYLASE, CPD), BRI1/BAK1 (BRASSINOSTEROID INSENSITIVE 1, BAK1), BIN2, CCD7/CCD8/D27/MAX (CCD7, CCD8, D27, MAX), D14/MAX2 (DWARF14 α/β HYDROLASE, MORE AXILLARY GROWTH 2), CKX (CYTOKININ OXIDASE/DEHYDROGENASE), IPT (ISOPENTENYL TRANSFERASE), PIF4/PIF5 (PIF4, PIF5), PHYB, HFR1 or BFT (BROTHER OF TFL AND FT), and/or orthologues thereof. In some embodiments, the improved agronomic efficiency may be increased yield per acre, increased harvesting efficiency, increased Brix in fruits of the Rubus plants, increased Brix:acidity ratio in fruits of Rubus plants, decreased water usage (e.g., a decrease in the amount of water used per amount of marketable fruit produced, decreased nitrogen usage (e.g., a decrease in the amount of nitrogen applied (amount of nitrogen applied per amount of marketable fruit harvested)), increased amount of marketable fruit produced and a reduction in the amount of unmarketable fruit produced, an increase in the amount of marketable fruit produced or an increase in the amount of marketable fruit harvested per pound of nitrogen applied.

In some embodiments a crop production system is provided comprising: (a) a population of Rubus plants having a compact growth habit and comprising at least one mutation in one or more endogenous genes that confer modifications in plant architecture; and (b) the population of Rubus plants is planted at a density of at least 4,000 plants per acre, wherein the system provides improved yield per unit area and reduced resource usage per pound of marketable fruit relative to a system comprising control Rubus plants. In some embodiments, a method of improving at least one agronomic trait in a Rubus plant is provided, comprising planting compact Rubus plants at a high density, wherein the compact Rubus plants comprise at least one mutation in one or more endogenous genes and exhibit increased apical dominance (increased determinacy), reduced height, reduced cane length, increased cane number, reduced lateral branching and/or reduced time to flowering, thereby resulting in at least one improved agronomic trait as compared to a control Rubus plant (devoid of the at least one mutation in the endogenous gene and does not exhibit compact growth). In some embodiments, the one or more endogenous genes are TFL (TERMINAL FLOWER), CEN (CENTRORADIALIS), BRC1 (BRANCHED1), AP1 (APETALA1), FT (FLOWERING LOCUS T), GA20ox/GA3ox (GIBBERELLIN 20-OXIDASE, GIBBERELLIN 3-OXIDASE), GA2ox (GIBBERELLIN 2-OXIDASE), GID1 (GIBBERELLIN INSENSITIVE DWARF1), DELLA (DELLA PROTEIN (GA SIGNALING REPRESSOR), SLY1/GID2 (SLEEPY1, GIBBERELLIN INSENSITIVE DWARF2), YUCCA (YUCCA FLAVIN MONOOXYGENASE-LIKE), PIN1/PIN3/PIN4 (PIN-FORMED 1 AUXIN TRANSPORTER, PIN3, PIN4), AUX1, BR1/BR2 (BR1, BR2), AUX/IAA (AUX, IAA), DWF4/CPD (DWARF4 STEROL C-22 HYDROXYLASE, CPD), BRI1/BAK1 (BRASSINOSTEROID INSENSITIVE 1, BAK1), BIN2, CCD7/CCD8/D27/MAX (CCD7, CCD8, D27, MAX), D14/MAX2 (DWARF14 α/β HYDROLASE, MORE AXILLARY GROWTH 2), CKX (CYTOKININ OXIDASE/DEHYDROGENASE), IPT (ISOPENTENYL TRANSFERASE), PIF4/PIF5 (PIF4, PIF5), PHYB, HFR1 or BFT (BROTHER OF TFL AND FT), and/or orthologues thereof. Accordingly, in some embodiments, a crop production system is provided comprising: (a) a population of Rubus plants comprising a mutation in an endogenous TERMINAL FLOWER (TFL) gene conferring compact growth; and (b) the population of Rubus plants is planted at a density of at least 4,000 plants per acre, wherein the system provides improved yield per unit area and reduced resource usage per pound of marketable fruit relative to a system comprising control Rubus plants.

In some embodiments, a method of improving at least one agronomic trait in a Rubus plant is provided, comprising planting Rubus plants at a high density, wherein the Rubus plants comprise at least one mutation in an endogenous TERMINAL FLOWER (TFL) gene encoding a TFL polypeptide, and exhibit a compact growth habit resulting in an improvement of at least one agronomic trait as compared to a control Rubus plant (devoid of the at least one mutation in the TFL gene and does not exhibit compact growth). In some embodiments, a method of improving at least one agronomic trait in a Rubus plant, comprising planting compact Rubus plants at a high density, wherein the compact Rubus plants comprise at least one mutation in an endogenous TERMINAL FLOWER (TFL) gene and exhibit increased apical dominance (increased determinacy), reduced height, reduced cane length, increased cane number, reduced lateral branching and/or reduced time to flowering, thereby resulting in at least one improved agronomic trait as compared to a control Rubus plant (devoid of the at least one mutation in the endogenous TFL gene and does not exhibit compact growth). In some embodiments, the at least one agronomic trait comprises increased yield per acre, increased harvesting efficiency, increased Brix in fruits of the Rubus plants, increased Brix:acidity ratio in fruits of Rubus plants, decreased water usage (e.g., a decrease in the amount of water used per amount of marketable fruit produced, decreased nitrogen usage (e.g., a decrease in the amount of nitrogen applied (amount of nitrogen applied per amount of marketable fruit harvested)), increased amount of marketable fruit produced and a reduction in the amount of unmarketable fruit produced, an increase in the amount of marketable fruit produced or an increase in the amount of marketable fruit harvested per pound of nitrogen applied.

In some embodiments, wherein the planting at a high density of Rubus plants comprising at least one mutation in an endogenous TFL gene encoding a TFL polypeptide, and exhibiting compact growth habit means planting at a density that is increased by about 100% to about 300% compared to Rubus plants devoid of the at least one mutation in an endogenous TFL gene. In some embodiments, wherein the planting at a high density of Rubus plants comprising at least one mutation in an endogenous TFL gene encoding a TFL polypeptide, and exhibiting compact growth habit means planting at a density that is that is increased by about 2 times to 3 times compared to Rubus plants devoid of the at least one mutation in an endogenous TFL gene. In some embodiments, high density means planting the Rubus plants at a density that is about 4000 plants per acre to about 7500 plants per acre. In some embodiments, planting at a high density of Rubus plants comprising at least one mutation in an endogenous TFL gene encoding a TFL polypeptide, and exhibiting compact growth habit means the Rubus plants are planted in rows that are about 3 foot to about 5.5 foot apart as compared to a row spacing of 6 ft to about 10 foot rows for Rubus plants devoid of the at least one mutation in an endogenous TFL gene.

In some embodiments, the yield per acre for the Rubus plants having the least one mutation in an endogenous TFL gene and exhibiting compact growth habit is about 5000 crates/acre to about 10,000 crates/acre as compared to about 4000 crates/acre to about 4500 crates/acre for Rubus plants not comprising the at least one mutation in an endogenous TFL gene and planted under standard growing practices. In some embodiments, the yield per acre for the Rubus plants having the least one mutation in an endogenous TFL gene and exhibiting compact growth habit is about 20,000 pounds (lbs)/acre to about 50,000 lbs/acre. In some embodiments, the yield per acre for the Rubus plants having the least one mutation in an endogenous TFL gene and exhibiting compact growth habit is about 30,000 pounds (lbs)/acre to about 90,000 lbs/acre. In some embodiments, the yield per acre for the Rubus plants having the least one mutation in an endogenous TFL gene and planted at a high density is about 25% to about 450% greater than that for Rubus plants that are devoid of the at least one mutation in an endogenous TFL gene and planted under standard growing practices, optionally wherein the yield per acre is about 100% to about 250% greater. In some embodiments, the yield per acre for the Rubus plants having the least one mutation in an endogenous TFL gene and planted at a high density is about 2 times to about 4 times greater than that for Rubus plants that are devoid of the at least one mutation in an endogenous TFL gene and planted under standard growing practices.

In some embodiments, the harvesting efficiency (harvested fruit weight per person minute) of a population of Rubus plants comprising the least one mutation in an endogenous TFL gene and exhibiting compact growth habit is increased by about 20% to about 30% as compared to a Rubus control plant when grown under high density planting conditions. In some embodiments, the Brix of the fruit of a Rubus plant comprising the least one mutation in an endogenous TFL gene and exhibiting compact growth habit is increased by about 10% to about 35% as compared to a control Rubus plant. In some embodiments, the Brix:acidity ratio of the fruit of a Rubus plant comprising the least one mutation in an endogenous TFL gene and exhibiting compact growth habit is increased by about 10% to about 60% as compared to a control Rubus plant. In some embodiments, the water usage (gallons of water used per pound of fruit produced) for Rubus plants comprising the least one mutation in an endogenous TFL gene and exhibiting compact growth habit is decreased by about 20% to about 90% as compared to a control Rubus plant. In some embodiments, for Rubus plants comprising the least one mutation in an endogenous TFL gene and exhibiting compact growth habit, the amount of nitrogen applied per amount of marketable fruit produced is decreased by about 20% to about 80% as compared to a control Rubus plant. In some embodiments, the amount of unmarketable fruit produced by is reduced by at least about 1% to about 2.5% as compared to the control. In some embodiments, Rubus plants comprising the least one mutation in an endogenous TFL gene and exhibiting compact growth habit the amount of marketable fruit produced by is increased by at least about 100% to about 200% as compared to the control. In some embodiments, Rubus plants comprising the least one mutation in an endogenous TFL gene and exhibiting compact growth habit the amount of marketable fruit harvested per pound of nitrogen applied may be increased by about 20% to about 125%.

In some embodiments, the at least one mutation in an endogenous TFL gene results in a truncated TFL polypeptide and/or no detectable TFL polypeptide. In some embodiments, the Rubus plant or part thereof may comprise at least two endogenous TFL genes and at least one allele of the at least two endogenous TFL genes may comprise the at least one mutation. In some embodiments, the Rubus plant comprising at least one mutation in an endogenous TERMINAL FLOWER (TFL) gene (e.g., one or more TFL genes) encoding a TFL polypeptide and exhibiting a compact growth habit may be more practicable commercially and agriculturally as compared to a control Rubus plant.

In some embodiments, a Rubus plant comprising at least one mutation in an endogenous TERMINAL FLOWER (TFL) gene (e.g., one or more TFL genes) encoding a TFL polypeptide and exhibiting a compact growth habit, when planted at a high density results in reduced resource utilization (less labor, less pruning, easier harvesting and trellising resulting in reduced labor costs), and reduced resource competition (less growth results in less demand for water and nutrients (e.g., reduced nitrogen) between plants; less susceptibility to disease and pests due to more open canopy).

In some embodiments, a Rubus plant having the least one mutation in an endogenous gene associated with plant architecture (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) does not require pruning or tipping. “High density” as used herein means a plant density that is increased by about 100% to about 550% as compared to control Rubus plants not comprising the at least one mutation in an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) and planted under standard growing practices. In some embodiments, planting density is increased by about 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, or 550% or any value or range therein (e.g., about 150% to about 175, 200, 225, 250, 275, 300, 325, 350, 375, 400, 425, 430, 435, 440, 445, 450, 475, 500, 525, or 550%; about 200% to about 250, 300, 325, 350, 375, 400, 425, 430, 435, 440, 445, 450, 475, 500, 525, or 550%; about 250% to about 255, 260, 265, 270, 275, 280, 285, 290, 295, 300, 325, 330, 335, 340, 345, 350, 355, 360, 365, 370, 375, 380, 385, 390, 395, 400, 425, 430, 435, 440, 445, 450, 475, 500, 525, or 550%, and the like). “High density” as used herein also means planting Rubus plants at a density that is about 4000 plants per acre to about 7500 plants per acre (e.g., about 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, or 7500 plants per acre, and any range or value therein), optionally about 4000 plants per acre to about 10,000 plants per acre (e.g., about 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 plants per acre, and any range or value therein) as compared to the number of plants per acre under standard growing conditions of about 1400 plants per acre to about 3000 plants per acre (e.g., about 1400, 1500, 1600, 1700, 1800, 1900, 2000, 2100, 2200, 2300, 2400, 2500, 2600, 2700, 2800, 2900, or 3000 plants per acre, and any range or value therein). In some embodiments, the planting density (plants per acre) of Rubus plants comprising at least one mutation in an endogenous TFL gene or any one or more of the endogenous genes of CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX IPT, PIF4/PIF5, PHYB, HFR1, BFT, or orthologues thereof (and exhibiting compact growth) may be in a range from about 4000 to about 6500, about 4000 to about 6000, about 4500 to about 7500, about 4500 to about 7000, about 4500 to about 6500, about 4500 to about 6000, about 5000 to about 7500, about 5000 to about 7000, about 5000 to about 6500, about 5000 to about 6000, about 6000 to about 9000, about 7000 to about 9000, about 8000 to about 10000, about 9000 to about 10,000 plants per acre and the like, and any range or value therein.

In some embodiments, a high density planting means planting Rubus plants at a density that is about 2 times to about 4 times (about 2 fold to about 3 fold) (e.g., about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 times or any range or value therein) the number of plants per acre under standard growing conditions. In some embodiments, when planted at a high density means the Rubus plants are planted in rows that are about 3 feet to about 5.5 feet apart (e.g., about 3, 3.25, 3.5, 3.75, 4, 4.25, 4.5, 4.75, 5, 5.25 or 5.5 ft), optionally about 3 ft to about 4 ft apart (e.g., about 3, 3.25, 3.5, 3.75, 4 ft), or about 3 ft to about 3.5 ft apart (3, 3.25, or 3.5 ft apart), with spacing between plants within each row being about 1 foot to about 2.5 feet (e.g., with spacing between plants with a row being about 1 foot, about 1.5 feet, about 2 feet, about 2.5 feet, e.g., 1, 1.25, 1.5, 1.75, 2, 2.25, 2.5 feet), optionally wherein the spacing between plants within each row may be about 1.5 feet, as compared to standard row spacing of about 6 feet to about 10 feet (e.g., about 6, 6.5, 7, 7.5, 8, 8.5, 9, 9.5 or 10 ft apart), optionally about 6 ft to about 8 ft (e.g., about 6, 6.5, 7, 7.5, or 8 ft apart) and spacing between plants within each row being about 2-5 feet (e.g., about 6, 6.5, 7, 7.5, 8, 8.5, 9, 9.5, 10 feet between rows with spacing of about 2, 2.5, 3, 3.5, 4, 4.5 or 5 feet between plants). In some embodiments, standard planting for blackberry may be about 6-8 foot rows and spacing between plants within each row being about 2-3 feet, resulting in about 1815 blackberry plants per acre as compared to about 3-3.5 ft rows for blackberry plants having as least one mutation in an endogenous TFL gene and exhibiting compact growth as described herein with spacing between the plants having the at least one TFL mutation being about 1.5-2 ft resulting in about 5800 or more plants per acre.

“Fruit quality” as used herein refers to the ratio of marketable to unmarketable fruit, the taste (Brix, acidity) and flavor score and can be as compared to a control Rubus plants not comprising the mutation(s) and not having compact growth, optionally wherein the control Rubus cannot be be planted at a high density without compromising plant health, yield or fruit quality.

In some embodiments, the at least one improved agronomic trait may be increased yield per acre or increased harvesting efficiency, when planted at a high density as described herein. In some embodiments, the at least one improved agronomic trait may be increased Brix and/or increased Brix:acidity ratio in the fruits of the Rubus plants having the least one mutation in an endogenous TFL gene and/or one or more of the other endogenous genes described herein (e.g., CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) and exhibiting compact growth. In some embodiments, the at least one improved agronomic trait may be decreased water usage such as a decrease in the amount of water used per amount of marketable fruit produced. In some embodiments, the at least one improved agronomic trait may be decreased nitrogen usage such as a decrease in the amount of nitrogen applied per amount of marketable fruit harvested. In some embodiments, the at least one improved agronomic trait may be a reduction in the amount of unmarketable fruit produced. In some embodiments, the at least one improved agronomic trait may be an increase in the amount of marketable fruit produced. In some embodiments, the at least one improved agronomic trait may be any combination of the improved agronomic traits described herein (e.g., a combination of 2, 3, 4, 5, 6, 7, or 8, or more different improved agronomic traits as described herein).

In some embodiments, the yield per acre for the Rubus plants having the least one mutation in an endogenous TFL gene and exhibiting compact growth, which are planted at a high density may be about 5000 crates/acre to about 10,000 crates/acre (e.g., about 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10,000 crates/acre, or any value or range therein, e.g., about 5000 to about 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, or 9000 crates/acre; about 6000 to about 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9500 or 10000 crates/acre; about 7000 to about 8000, 9000 or 10,000 crates/acre; about 8000 to about 9000 or 10,000 crates/acre and the like) as compared to about 4000 crates/acre to about 4500 crates/acre for control Rubus plants not comprising the at least one mutation in an endogenous TFL gene and planted under standard growing practices. A crate of berries weighs about 5 lbs.

In some embodiments, the yield per acre for the Rubus plants having the least one mutation in an endogenous TFL gene and exhibiting compact growth may be about 25% to about 450% (e.g., about 25, 30, 35, 40, 45, 50, 55, 55, 60, 65, 70, 75, 80, 85, 90, 95, 100, 110, 115, 120, 125, 130, 135, 140, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, or 450% or any range or value therein; e.g., about 50% to about 450%, about 100% to about 450%, about 125% to about 450%, about 150% to about 450%, about 200% to about 450%, about 100% to about 400%, about 125% to about 400%, about 150% to about 400%, about 100% to about 350%, about 125% to about 350%, about 150% to about 350%, about 100% to about 300%, about 125% to about 300%, about 150% to about 300%, about 100% to about 250%, about 125% to about 250%, about 150% to about 250%, and the like) higher than that of a Rubus plant devoid of the at least one mutation in an endogenous TFL gene. In some embodiments, the yield per acre for Rubus plants having the least one mutation in an endogenous TFL gene and exhibiting compact growth may be about 1 times to about 3 times (e.g., about 1, 1.25, 1.5, 1.75, 2, 2.25, 2.5, 2.75, or 3 times greater) greater than that of a Rubus plant devoid of the at least one mutation in an endogenous TFL gene.

In some embodiments, the yield per acre for the Rubus plants having the least one mutation in an endogenous TFL gene, which are planted at a high density may be about 20,000 pounds (lbs)/acre to about 50,000 lb/acre (20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 23,500, 34,000, 34,500, 35,000, 35,500, 36,000, 36,500, 37,000, 37,500, 38,000, 38,500, 39,000, 39,500, 40,000, 40,500, 41,000, 41,500, 42,000, 42,500, 43,000, 43,500, 44,000, 44,500, 45,000, 45,500, 46,000, 46,500, 47,000, 47,500, 48,000, 48,500, 49,000, 49,500, and/or 50,000 lb/acre or any value or range therein; e.g., about 20,000 to about 30,000, about 20,000 to about 40,000, about 30,000 to about 40,000, about 30,000 to about 50,000 and/or about 40,000 to about 50,000 lb/acre or any value or range therein) as compared to about 9000 lbs/acre to about 15,000 lbs/acre (e.g., about 9000, about 10,000, about 11,000, about 12,000, about 13000, about 14,000, and/or about 15,000 lbs/acre; e.g., about 9000 to about 11,000, about 9000 to about 12,000, about 9000 to about 13,000, about 9000 to about 14,000 lbs/acre, about 10,000 to about 11,000, about 10,000 to about 12,000, about 10,000 to about 13,000, about 10,000 to about 14,000, about 12,000 to about 13,000, about 12,000 to about 14,000 lbs/acre) for Rubus plants not comprising the at least one mutation in an endogenous TFL gene and planted under standard growing practices. In some embodiments, the yield per acre for the Rubus plants having the least one mutation in an endogenous TFL gene when planted at a high density may be about 100% to about 250% (e.g., about 100, 110, 115, 120, 125, 130, 135, 140, 150, 155, 160, 165, 170, 175, 180, 185, 190, 195, 200, 205, 210, 215, 220, 225, 230, 235, 240, 245, or 250% or any range or value therein; e.g., about 100% to about 200%, about 100% to about 150%, about 125% to about 250%, about 125% to about 200%, about 150% to about 250%, and the like) higher. In some embodiments, when planted at high density, the yield per acre (e.g., lbs/acre) for the Rubus plants having the least one mutation in an endogenous TFL gene and exhibiting compact growth may be about 2 to about 4 times greater (e.g., about 2, 2.25, 2.5, 2.75, 3, 3.25, 3.5, 3.75, 4 times greater) than that for Rubus plants not comprising the at least one mutation in an endogenous TFL gene and planted under standard growing practices.

In some embodiments, the yield per acre for the Rubus plants having the least one mutation in an endogenous TFL gene, which are planted at a high density may be about 30,000 to about 90,000 lbs/acre (e.g., about 30,000, about 35,000, about 40,000, about 45,000, about 60,000, about 65,000, about 70,000, about 80,000, about 85,000, or about 90,000 lbs/acre, and any range or value therein; e.g., about 30,000 lbs/acre to about 50,000 lbs/acre, about 30,000 lbs/acre to about 60,000 lbs/acre, about 30,000 lbs/acre to about 70,000 lbs/acre, about 40,000 lbs/acre to about 60,000 lbs/acre, about 40,000 lbs/acre to about 70,000 lbs/acre, about 40,000 lbs/acre to about 80,000 lbs/acre, about 40,000 lbs/acre to about 90,000 lbs/acre, and any range or value therein) as compared to about 15,000 lbs/acre to about 22,000 lbs/acre (e.g., about 15,000, about 16,000, about 17,000, about 18,000, about 19,000, about 20,000, about 21,000 and/or about 22,000 lbs/acre; e.g., about 15,000 lbs/acre to about 18,000 lbs/acre, about 15,000 lbs/acre to about 20,000 lbs/acre, about 16,000 lbs/acre to about 20,000 lbs/acre, about 16,000 lbs/acre to about 22,000 lbs/acre, about 18,000 lbs/acre to about 20,000 lbs/acre, about 18,000 lbs/acre to about 22,000 lbs/acre, about 20,000 lbs/acre to about 22,000 lbs/acre) for Rubus plants not comprising the at least one mutation in an endogenous TFL gene and planted under standard growing practices.

In some embodiments, harvesting efficiency (e.g., harvested fruit weight (lbs) per person per minute spent harvesting) of Rubus plants having the least one mutation in an endogenous TFL gene and exhibiting compact growth may be increased by about 20% to about 30% (e.g., an increase in harvesting efficiency of about 20, 21, 22, 23, 24, 25, 26, 27, 28, 29, or 30%) as compared to a control.

In some embodiments, the Brix of the fruits of the Rubus plants having the least one mutation in an endogenous TFL gene and exhibiting compact growth may be increased by about 10% to about 35% (e.g., about 10, 11, 12, 13, 14, 15, 16, 17, 18, 19, 20, 21, 22, 23, 24, 25, 26, 27, 28, 29, 30, 31, 32, 33, 34, or 35%) as compared to a control. In some embodiments, the Brix:acidity ratio of the fruits of the Rubus plants having the least one mutation in an endogenous TFL gene and exhibiting compact growth may be increased by about 10% to about 60% (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60%) as compared to a control.

In some embodiments, water usage for Rubus plants having the least one mutation in an endogenous TFL gene and exhibiting compact growth (gallons of water used per pound of fruit produced) may be decreased by about 20% to about 90% (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90%) as compared to a control.

In some embodiments, nitrogen usage (the amount of nitrogen applied per amount of marketable fruit harvested) for Rubus plants having the least one mutation in an endogenous TFL gene and exhibiting compact growth may be decreased by about 20% to about 80% (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80%) as compared to a control.

In some embodiments, the amount of unmarketable fruit produced (pounds per acre) may be reduced by about 1% to about 2.5% (e.g., about 1, 1.25, 1.5, 1.75, 2, 2.25 or 2.5%) in Rubus plants having the least one mutation in an endogenous TFL gene and exhibiting compact growth as compared to a control.

In some embodiments, the amount of marketable fruit produced (pounds per acre) by Rubus plants having the least one mutation in an endogenous TFL gene and exhibiting compact growth may be increased by about 100% to about 200% (e.g., about 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200%) as compared to a control.

In some embodiments, the amount of marketable fruit harvested per pound of nitrogen applied for Rubus plants having the least one mutation in an endogenous TFL gene and exhibiting compact growth may be increased by about 20% to about 125% (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125%) as compared to a control.

In addition to trellising, regular pruning, known as “tipping” is necessary to manage the growth of a Rubus plant not having the mutation in the at least one endogenous gene as described herein (to maintain their height, increase yield and to keep them from getting leggy), and for ease of harvesting. “Tipping” typically occurs in the spring or early summer as the canes are developing before the onset of flowering. Notably, and as a further advantage over control Rubus plants (e.g., a Rubus plant of the same species that does not comprise the same mutation), Rubus plants of this invention that can be planted at a high density resulting in increased yield, do not require tipping to control their growth.

Types of mutations useful for production of Rubus plants having compact growth and having an improvement in at least one agronomic trait (e.g., yield per acre, harvesting efficiency, Brix, Brix/acidity ratio, resource usage and fruit quality) (optionally when planted at a high density) as compared to Rubus plants devoid of the mutation in one or more of the endogenous genes as described herein, include substitutions, deletions and/or insertions. In some embodiments, mutations useful for producing a phenotype of increased yield per acre when planted at high density as compared to a control Rubus plant not comprising the mutation in one or more of the endogenous gene(s) as described herein may be generated by mutations that result in truncated genes or truncated polypeptides encoded by the endogenous gene. In some aspects, a mutation in an endogenous gene as described herein may be a hypomorphic mutation. or entirely deleted the polypeptides encoded by the endogenous gene. Such a mutation may be a null mutation.

In some embodiments, a Rubus plant or plant part thereof is provided, the Rubus plant or plant part thereof comprising at least one mutation (e.g., 1, 2, 3, 4, 5, or more mutations) in at least one copy of an endogenous gene including, but not limited to, TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) (e.g., in 1, 2, 3, 4 or more copies of such genes) and exhibiting a compact growth habit. In some embodiments, at least one mutation in the Rubus plant or plant part may be a null allele. In some embodiments, at least one mutation in the Rubus plant or plant part may be a dominant-negative mutation, semi-dominant mutation, weak loss of function mutation, or a hypomorphic mutation. In some embodiments, at least one mutation in the Rubus plant or plant part may be a non-natural mutation.

In some embodiments, a Rubus plant or plant part thereof is provided, the Rubus plant or plant part thereof comprises at least one mutation (e.g., 1, 2, 3, 4, 5, or more mutations) in at least one copy of an endogenous gene encoding a TFL polypeptide (e.g., in 1, 2, 3, 4 or more copies) and exhibits a compact growth habit. In some embodiments, a Rubus plant or plant part may comprise 1, 2, 3, or 4 TFL alleles and one or more (e.g., 1, 2, 3, or 4) may be mutated as described herein, optionally wherein all TFL alleles of the Rubus plant comprise one or more mutation as described herein. In some embodiments, at least one mutation in the Rubus plant or plant part may be a null allele of a TFL gene. In some embodiments, at least one mutation in an endogenous TFL gene in a Rubus plant or plant part may be a dominant-negative mutation, semi-dominant mutation, weak loss of function mutation, or a hypomorphic mutation. In some embodiments, at least one mutation in an endogenous TFL gene in the Rubus plant or plant part may be a non-natural mutation.

In some embodiments, a mutation in an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) may provide Rubus plants having a more compact growth habit as compared to a control Rubus plant. Rubus plants not comprising a mutation in an endogenous gene as described herein may display taller growth habit with a longer juvenile period before initiating flowering, which then terminates the vegetative growth period.

A plant having a “compact growth habit” means the plant comprises an architecture that is shorter than a wild type Rubus plant or a control Rubus plant not comprising a mutation(s) as described herein. A Rubus plant having a compact growth habit may have increased apical dominance (increased determinacy), a reduced height, reduced/shortened stem length, reduced cane length, increased number of canes, reduced lateral branching, and/or more determinate flowering (shorter time to flowering) as compared to a control Rubus plant not comprising the mutation as described herein. A Rubus plant having a compact growth habit typically requires less pruning than either a control or wild type Rubus plant.

In the case of Rubus plants useful with methods of the present invention, pruning or tipping is not required due to their compact growth habit. In some embodiments, a Rubus plant having at least one mutation in a TEL gene or a an edited gene ofany one or more of CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BET, or orthologue thereof, and exhibiting a compact growth habit may have a height of 5 feet or less (e.g., 2.5, 3, 3.25, 3.5, 3.75, 4, 4.25, 4.5, 4.75 or 5 feet tall) or a height of 150 cm or less (e.g., 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 cm).

In some embodiments, in addition to modification of TERMINAL FLOWER (TEL) genes, compact growth habit in Rubus may be achieved by modification of any one or more of the genes (or orthologues thereof) provided below in Table 1.

TABLE 1
Endogenous genes of interest for modifying Rubus as described herein.

| | Gene(s) | | |
|---|---|---|---|
| Pathway | of Interest | Gene Names | Mechanism |
| Flowering and | CEN | CENTRORADIALIS | Loss of function promotes |
| Architecture | | | determinate growth habit |
| Regulation | | | |
| Flowering and | BRC1 | BRANCHED1 | Increase expression to arrest |
| Architecture | | | axillary bud development, create |
| Regulation | | | more compact canopy |
| Flowering and | AP1 | APETALA1 | Mild upregulation to promote |
| Architecture | | | transition to flowering |
| Regulation | | | |
| Flowering and | FT | FLOWERING | Upregulate to promote transition |
| Architecture | | LOCUS T | to flowring |
| Regulation | | | |
| GA Biosynthesis | GA20ox/ | GIBBERELLIN 20- | Shorter internodes from reduced |
| and Signalling | GA3ox | OXIDASE, | GA synthesis |
| | | GIBBERELLIN 3- | |
| | | OXIDASE | |
| GA Biosynthesis | GA2ox | GIBBERELLIN 2- | Shorter internodes from |
| and Signalling | | OXIDASE | increased GA deactivation |
| GA Biosynthesis | GID1 | GIBBERELLIN | Decrease GA perception for |
| and Signalling | | INSENSITIVE | semi-dwarf phenotype |
| | | DWARF1 | |
| GA Biosynthesis | DELLA | DELLA PROTEIN (GA | Increase expression to restrain |
| and Signalling | | SIGNALING | growth |
| | | REPRESSOR) | |
| GA Biosynthesis | SLY1/ | SLEEPY1, | Prevent DELLA turnover to |
| and Signalling | GID2 | GIBBERELLIN | restrain growth |
| | | INSENSITIVE | |
| | | DWARF2 | |
| Auxin | YUCCA | YUCCA FLAVIN | Decrease IAA for shorter |
| Biosynthesis and | | MONOOXYGENASE- | internodes |
| Transport | | LIKE | |
| Auxin | PIN1/PIN3/ | PIN-FORMED 1 AUXIN | Reduce auxin transport, limiting |
| Biosynthesis and | PIN4 | TRANSPORTER, PIN3, | vegetative elongation |
| Transport | | PIN4 | |
| Auxin | AUX1 | AUX1 | Limit auxin transport for compact |
| Biosynthesis and | | | growth habit |
| Transport | | | |
| Auxin | BR1/BR2 | BR1, BR2 | Limit auxin transport for compact |
| Biosynthesis and | | | growth habit |
| Transport | | | |
| Auxin | AUX/IAA | AUX, IAA | Reduce auxin response for |
| Biosynthesis and | | | dwarfing effect |
| Transport | | | |
| Brassinosteroid | DWF4/ | DWARF4 STEROL C- | Reduce BR synthesis for |
| Biosynthesis and | CPD | 22 HYDROXYLASE, | compact phenotype |
| Signalling | | CPD | |
| Brassinosteroid | BRI1/ | BRASSINOSTEROID | Limit BR perception for compact |
| Biosynthesis and | BAK1 | INSENSITIVE 1, BAK1 | phenotype |
| Signalling | | | |
| Brassinosteroid | BIN2 | BIN2 | Increase expression to limit BR |
| Biosynthesis and | | | signalling for dwarfing effect |
| Signalling | | | |
| Strigolactone | CCD7/ | CCD7, CCD8, D27, | Limit SL production for shorter |
| Biosynthesis and | CCD8/D27/ | MAX | plants with more laterals |
| Signalling | MAX | | |
| Strigolactone | D14/MAX2 | DWARF14 α/β | Limit SL perception for compact, |
| Biosynthesis and | | HYDROLASE, MORE | branched phenotype |
| Signalling | | AXILLARY GROWTH 2 | |
| Cytokinin | CKX | CYTOKININ | Increase expression to reduce |
| Metabolism and | | OXIDASE/ | CK, reducing intenode |
| Signalling | | DEHYDROGENASE | elongation |
| Cytokinin | IPT | ISOPENTENYL | Reduce BK synthesis for |
| Metabolism and | | TRANSFERASE | semidwarf phenotype |
| Signalling | | | |
| Photomorphogenesis | PIF4/PIF5 | PIF4, PIF5 | Weaken expression to suppress |
| and Shade | | | elongation |
| Avoidance | | | |
| Photomorphogenesis | PHYB | PHYB | Increase expression to limit |
| and Shade | | | shade avoidance response |
| Avoidance | | | |
| Photomorphogenesis | HFR1 | HFR1 | Increase expression to reduce |
| and Shade | | | internode elongation |
| Avoidance | | | |

Compact, determinate, or semi-dwarf phenotypes can arise from alterations in diverse regulatory pathways, including those governing flowering time, shoot architecture, hormonal signaling, and axillary branching. Exemplary gene classes and representative targets are summarized in Table 1. These genes have been associated in other species (e.g., maize, rice, Arabidopsis) with traits such as reduced plant height, shorter internodes, decreased apical dominance, and improved harvest index. Orthologs of these genes in Rubus are identifiable using standard bioinformatic and molecular techniques as known in the art and as described below and may be modified/edited as described herein.

Each of the gene families as provided in Table 1 may influence plant stature or architecture through distinct mechanisms, including modulation of meristem determinacy, hormone biosynthesis or signaling, and cellular elongation processes. Modulating these targets in Rubus, whether by knockout, knockdown, base editing, or overexpression, can produce phenotypes characterized by shorter internodes, reduced lateral branching, or determinate flowering. Such traits permit increased planting density as described herein without excessive canopy overlap, thereby enhancing land-use efficiency and improving one or more agronomic parameters, including yield per acre, harvest efficiency, and water or nitrogen-use efficiency.

In some embodiments, Rubus plants comprising at least one mutation in an endogenous gene as described herein, and exhibiting a compact growth habit when planted at a high density may be practicable both commercially and agriculturally when compared to a control Rubus plant planted at a high density. In some embodiments, the Rubus plant comprising at least one mutation in an endogenous gene as described herein, and exhibiting a compact growth habit, when planted at a high density as described herein results in reduced resource utilization (less labor, less pruning, easier harvesting and trellising resulting in reduced labor costs), and reduced resource competition (less growth results in less demand for water and nutrients between plants); less susceptibility to disease and pests due to more open canopy). In some embodiments, the Rubus plants of the present invention may also show additionally improved agronomic characteristics as compared to control Rubus plants even when planted at a standard planting density as described herein.

A mutation in an endogenous gene as described herein, e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof, of a Rubus plant or plant part thereof may be any type of mutation, including a base substitution, a base deletion, and/or a base insertion, optionally wherein the mutation is a point mutation. Thus, in some embodiments, a mutation in a TFL gene of a Rubus plant, part thereof may be any type of mutation, including a base substitution, a base deletion, and/or a base insertion, optionally wherein the mutation is a point mutation. In some embodiments, a Rubus plant or part thereof may comprise one or more endogenous TFL genes and one or more alleles of the one or more genes may each comprise a mutation as described herein (see also, WO2023/192838 for its discussion of mutations in TFL).

A mutation in an endogenous gene as described herein in a Rubus plant, plant part or plant cell may be any mutation as described herein, including a base deletion, base substitution or base insertion. In some embodiments, the at least one mutation may a dominant negative mutation, a semi-dominant mutation, a weak loss of function mutation, a null mutation, or a hypomorphic mutation, optionally, wherein the at least one mutation results in a null allele. In some embodiments, at least one mutation may be a non-natural mutation. In some embodiments, the mutation in an endogenous gene may be a hypomorphic mutation and results in an amino acid substitution within the encoded polypeptide. In some embodiments, the mutation may be a truncation or deletion that results in a truncation of the encoded protein, which may result in no detectable protein (e.g., a null mutation, null allele or knockout).

In some embodiments, an endogenous gene as described herein may be mutated using an editing system. The nucleic acid binding domain of an editing system useful with this invention may be from a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, a nuclease useful with the invention is a zinc finger nuclease, a transcription activator-like effector nuclease (TALEN), an endonuclease (e.g., Fok1) or a CRISPR-Cas effector protein. In some embodiments, a mutation of an endogenous TFL gene may be made following cleavage by an editing system that comprises a nuclease and a nucleic acid binding domain that binds to a target site within a sequence having least 80% sequence identity to a sequence encoding of any one of SEQ ID NOs:72, 73, 106, 107, 114, 115, 126, 127, 140, 141, 153, 162, 163, 172, 173, 184, 185, 214, 215, 234, 235, or 252, or having at least 80% sequence identity to a sequence encoding any one of the amino acid sequences of SEQ ID NOs:74, 108, 116, 128, 142, 154, 164, 174, 186, 216, 236 or 253, and the at least one mutation within a TFL gene is made following cleavage by the nuclease, optionally wherein the mutation may be a non-natural mutation.

In some embodiments, a Rubus plant part/plant cell edited as described herein may be regenerated into a Rubus plant, thereby providing a Rubus plant with a mutation in one or more of the endogenous genes as described herein and which exhibits a compact growth habit as compared to a Rubus plant not comprising the mutation in the one or more of the endogenous genes as described herein and that does not exhibit a compact growth habit.

In some embodiments, a Rubus plant part/plant cell edited as described herein may be regenerated into a Rubus plant, thereby providing a Rubus plant with a mutation in a TFL gene and which exhibits a compact growth habit as compared to a Rubus plant not comprising the mutation in the TFL gene.

A Rubus plant, part thereof, or cell useful with this invention may be (or may be from) any caneberry plant, optionally a blackberry plant, a red raspberry plant or a black raspberry plant.

In some embodiments, a method of producing/breeding a transgene-free edited Rubus plant is provided, comprising: crossing a Rubus plant of the present invention (e.g., a Rubus plant comprising a mutation in a TFL protein/gene or a mutation in any one or more of CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof and exhibiting a compact growth habit) with a transgene free Rubus plant, thereby introducing the at least one mutation into the Rubus plant that is transgene-free; and selecting a progeny Rubus plant that comprises the at least one mutation and is transgene-free, thereby producing a transgene free edited Rubus plant, optionally the at least one mutation may be a non-natural mutation.

Also provided is a method of providing a plurality of Rubus plants exhibiting a compact growth habit comprising planting two or more Rubus plants of the invention (e.g., 2, 3, 4, 5, 6, 7, 8, 9, 10 or more Rubus plants comprising a mutation in an endogenous gene as described herein and exhibiting a compact growth habit) in a growing area (e.g., a field (e.g., a cultivated field, an agricultural field), a growth chamber, a greenhouse, a recreational area, a lawn, and/or a roadside and the like), thereby providing a plurality of Rubus plants having a compact growth habit.

In some embodiments, a Rubus plant (or a plurality of Rubus plants of this invention) may be selfed and/or may be outcrossed with another Rubus plant.

In some embodiments, a method for editing a specific site in the genome of a Rubus plant cell, the method comprising: cleaving, in a site specific manner, a target site within an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof) in the Rubus plant cell. In some embodiments, the edit results in a mutation, including but not limited to a deletion, substitution, or insertion. In some embodiments, the edit may be a nucleotide substitution to an A, a T, a G, or a C. In some embodiments, the edit results in a non-natural mutation. In some embodiments, the edit may result in a null allele, a dominant negative mutation, a semi-dominant mutation, a weak loss of function mutation, a null mutation (knock-out), or a hypomorphic mutation. In some embodiments, the edit is a deletion that results in a truncation of the encoded polypeptide or no detectable encoded polypeptide (e.g., a null mutation or knock-out).

In some embodiments, the method for editing may further comprise regenerating a Rubus plant from the Rubus plant cell comprising the edit in the endogenous gene as described herein, thereby producing a Rubus plant comprising the edit in the endogenous gene and exhibiting a compact growth habit.

In some embodiments, a method for editing a specific site in the genome of a Rubus plant cell is provided, the method comprising: cleaving, in a site specific manner, a target site within an endogenous TFL gene in the Rubus plant cell, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby generating an edit in the endogenous TFL gene of the Rubus plant cell and producing a plant cell comprising the edit in the endogenous TFL gene. In some embodiments, the edit results in a mutation, including but not limited to a deletion, substitution, or insertion. In some embodiments, the edit may be a nucleotide substitution to an A, a T, a G, or a C. In some embodiments, the edit results in a non-natural mutation. In some embodiments, the edit may result in a null allele, a dominant negative mutation, a semi-dominant mutation, a weak loss of function mutation, a null mutation (knock-out), or a hypomorphic mutation. In some embodiments, the edit is a deletion that results in a truncation of the TFL polypeptide or no detectable TFL polypeptide as described herein (e.g., a null mutation or knock-out).

In some embodiments, the method for editing may further comprise regenerating a Rubus plant from the Rubus plant cell comprising the edit in the endogenous TFL gene, thereby producing a Rubus plant comprising the edit in the endogenous TFL gene and exhibiting a compact growth habit.

In some embodiments, a method for making a Rubus spp. having a compact growth habit, optionally resulting in the improvement of at least one agronomic trait as described herein, is provided, the method comprising: (a) contacting a population of Rubus plant cells comprising at least one endogenous target gene, with a nuclease linked to a nucleic acid binding domain (e.g., an editing system) that binds to a target site in the at least one endogenous target gene, wherein the at least one endogenous target gene is TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof; (b) selecting a Rubus plant cell from said population that comprises a mutation in the at least one endogenous target gene; and (c) growing the selected Rubus plant cell into a Rubus plant, optionally wherein the mutation in the at least one endogenous target gene results in a null allele of the endogenous target gene. In some embodiments, the mutation in the at least one endogenous target gene may be a non-natural mutation.

In some embodiments, a method for making a Rubus spp. having a compact growth habit, optionally resulting in the improvement of at least one agronomic trait as described herein, is provided, the method comprising: (a) contacting a population of Rubus plant cells comprising at least one endogenous TFL gene with a nuclease linked to a nucleic acid binding domain (e.g., an editing system) that binds to a target site in the at least one endogenous TFL gene, wherein the at least one endogenous TFL gene: (i) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (ii) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (iii) encodes an amino acid sequence having at least 80% sequence identity to any one of SEQ ID NOs:74, 108, 116, 128, 142, 154, 164, 174, 186, 216, 236 or 253, and/or (iv) encodes a region having at least 80% sequence identity to any one of the amino acid sequences of SEQ ID NOs:193-197, 247, 248, or 271-276; (b) selecting a Rubus plant cell from said population that comprises a mutation in the at least one endogenous TFL gene; and (c) growing the selected Rubus plant cell into a Rubus plant, optionally wherein the mutation in the at least one endogenous TFL gene results in a null allele of the endogenous TFL gene. In some embodiments, the mutation in the at least one endogenous TFL gene may be a non-natural mutation.

In some embodiments, a method is provided for producing a Rubus plant or part thereof having comprising at least one cell having an endogenous gene with a mutation as described herein, and optionally comprising in the improvement of at least one agronomic trait as described herein, the method comprising contacting a target site in an endogenous gene in the Rubus plant or part with a nuclease comprising a cleavage domain and a DNA-binding domain, wherein the endogenous gene is TFL, CEN, BRC1, AP1, FT, GA20ox GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX IPT, PIF4/PIF5, PHYB, HFR1, BFT, or an orthologue thereof, and the nucleic acid binding domain binds to a target site in the endogenous gene, thereby producing the Rubus plant or part thereof comprising at least one cell having an endogenous gene of any one of TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof, with a mutation and exhibiting a compact growth habit.

In some embodiments, a method is provided for producing a Rubus plant or part thereof having comprising at least one cell having an endogenous TFL gene with a mutation, a compact growth habit, optionally comprising an improvement of at least one agronomic trait as described herein, the method comprising contacting a target site in an endogenous TFL gene in the Rubus plant or part with a nuclease comprising a cleavage domain and a DNA-binding domain, wherein the nucleic acid binding domain binds to a target site in the endogenous TFL gene, wherein the endogenous TFL gene (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276, thereby producing the Rubus plant or part thereof comprising at least one cell having an endogenous TFL gene with a mutation and exhibiting a compact growth habit.

In some embodiments, a Rubus plant or part thereof produced by the methods of this invention is provided that comprises at least one mutation in an endogenous gene as described herein and exhibits a compact growth habit resulting in at least one improved agronomic trait as described herein, optionally the improved agronomic trait is observed when the Rubus plant comprising the at least one mutation and a compact growth habit is planted at a high density, wherein the improvement is as compared to a control Rubus plant grown under the same conditions.

In some embodiments, a nuclease may cleave an endogenous gene as described herein, thereby introducing the mutation into the endogenous gene. A nuclease useful with the invention may be any nuclease that can be utilized to edit/modify a target nucleic acid. Such nucleases include, but are not limited to a zinc finger nuclease, transcription activator-like effector nucleases (TALEN), endonuclease (e.g., Fok1) and/or a CRISPR-Cas effector protein. Likewise, any nucleic acid binding domain useful with the invention may be any DNA binding domain or RNA binding domain that can be utilized to edit/modify a target nucleic acid. Such nucleic acid binding domains include, but are not limited to, a zinc finger, transcription activator-like DNA binding domain (TAL), an argonaute and/or a CRISPR-Cas effector DNA binding domain.

In some embodiments, a nucleic acid binding domain (e.g., DNA binding domain) is comprised in a nucleic acid binding polypeptide. A “nucleic acid binding protein” or “nucleic acid binding polypeptide” as used herein refers to a polypeptide that binds and/or is capable of binding a nucleic acid in a site- and/or sequence-specific manner. In some embodiments, a nucleic acid binding polypeptide may be a sequence-specific nucleic acid binding polypeptide (e.g., a sequence-specific DNA binding domain) such as, but not limited to, a sequence-specific binding polypeptide and/or domain from, for example, a polynucleotide-guided endonuclease, a CRISPR-Cas effector protein (e.g., a CRISPR-Cas endonuclease), a zinc finger nuclease, a transcription activator-like effector nuclease (TALEN) and/or an Argonaute protein. In some embodiments, a nucleic acid binding polypeptide comprises a cleavage polypeptide (e.g., a nuclease polypeptide and/or domain) such as, but not limited to, an endonuclease (e.g., Fok1), a polynucleotide-guided endonuclease, a CRISPR-Cas endonuclease, a zinc finger nuclease, and/or a transcription activator-like effector nuclease (TALEN). In some embodiments, the nucleic acid binding polypeptide associates with and/or is capable of associating with (e.g., forms a complex with) one or more nucleic acid molecule(s) (e.g., forms a complex with a guide nucleic acid as described herein) that can direct or guide the nucleic acid binding polypeptide to a specific target nucleotide sequence (e.g., a gene locus of a genome) that is complementary to the one or more nucleic acid molecule(s) (or a portion or region thereof), thereby causing the nucleic acid binding polypeptide to bind to the nucleotide sequence at the specific target site. In some embodiments, the nucleic acid binding polypeptide is a CRISPR-Cas effector protein as described herein. In some embodiments, reference is made to specifically to a CRISPR-Cas effector protein for simplicity, but a nucleic acid binding polypeptide as described herein may be used. In some embodiments, a polynucleotide and/or a nucleic acid construct of the invention can be an “expression cassette” or can be comprised within an expression cassette.

A mutation provided by methods of the invention may be a substitution, an insertion and/or a deletion, optionally wherein the insertion or deletion is a frameshift mutation, e.g., an in-frame insertion or in-frame deletion or an out-of-frame insertion or out-of-frame deletion. In some embodiments, the mutation may be a deletion of about 1 base pair, optionally about 3 consecutive base pairs to about 519 consecutive base pairs or about 3 consecutive base pairs to about 2600 consecutive base pairs, or any value or range therein. In some embodiments, the mutation may be a non-natural mutation. See, e.g., WO2023/192838 for its discussion of mutations in, for example, a TFL gene.

In another aspect, Rubus plants or plant part produced by the methods of the invention are provided that comprise in their genome one or more mutated endogenous TFL genes as described herein. In some embodiments, the mutated endogenous TFL gene comprises a non-natural mutation.

In some embodiments, the present invention provides a method of producing a Rubus plant comprising a mutation in an endogenous gene as described herein and at least one polynucleotide of interest, the method comprising crossing a Rubus plant of the invention comprising at least one mutation in the endogenous gene (a first Rubus plant) with a second Rubus plant that comprises the at least one polynucleotide of interest to produce progeny plants; and selecting progeny plants comprising at least one mutation in the endogenous gene and the at least one polynucleotide of interest, thereby producing the Rubus plant comprising a mutation in an endogenous gene as described herein and at least one polynucleotide of interest.

The present invention further provides a method of producing a Rubus plant comprising a mutation in an endogenous gene as described herein and at least one polynucleotide of interest, the method comprising introducing at least one polynucleotide of interest into a Rubus plant of the present invention comprising at least one mutation in an endogenous gene, thereby producing the Rubus plant comprising at least one mutation in an endogenous gene and at least one polynucleotide of interest.

In some embodiments, the present invention provides a method of producing a Rubus plant comprising a mutation in an endogenous gene as described herein and at least one polynucleotide of interest, the method comprising introducing at least one polynucleotide of interest into a Rubus plant of the invention comprising at least one mutation in an endogenous gene, thereby producing the Rubus plant comprising at least one mutation in an endogenous gene and at least one polynucleotide of interest.

In some embodiments, also provided is a method of producing a Rubus plant comprising a mutation in an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof) and exhibiting a compact growth habit that results in at least one improved agronomic trait as described herein, the method comprising crossing a first Rubus plant, which is a Rubus plant of the present invention comprising at least one mutation in the endogenous gene as described herein and exhibiting a compact growth habit, with a second Rubus plant; and selecting progeny plants comprising the mutation in the endogenous gene and exhibiting a compact growth habit and resulting in at least one improved agronomic trait as described herein, thereby producing the Rubus plant comprising a mutation in the endogenous gene resulting in at least one improved agronomic trait as described herein as compared to a control Rubus plant.

A TFL gene useful with this invention includes any TFL gene that when mutated as described herein (see also WO2023/192838) can confer a more compact growth habit (as compared to a control plant not comprising the mutation). Such TFL genes include, but are not limited to, a TFL gene (a) comprising a nucleotide sequence having at least 80% % sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:72, 73, 106, 107, 114, 115, 126, 127, 140, 141, 153, 162, 163, 172, 173, 184, 185, 214, 215, 234, 235, or 252, (b) comprising a region having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:75-105, 109-113, 117-125, 129-139, 143-152, 155-161, 165-171, 175-183, 187-192, 217-233, 237-246, or 254-270, (c) encoding a sequence having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of SEQ ID NOs:74, 108, 116, 128, 142, 154, 164, 174, 186, 216, 236 or 253, and/or (d) encoding a region having at least 80% sequence identity (e.g., 80, 81, 82, 83, 84, 85, 86, 87, 88, 89, 90, 91, 92, 93, 94, 95, 96, 97, 98, 99, or 100%, optionally about 90, 91, 92, 93, 94, 95, 96, 97, 98, 99, or 100% or about 95, 96, 97, 98, 99, or 100% sequence identity) to any one of the amino acid sequences of SEQ ID NOs:193-197, 247, 248, or 271-276.

Any mutation in a endogenous gene of this invention (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof) that produces Rubus plants or parts thereof having a compact growth habit and when planted at a high density results in at least one improved agronomic trait as described herein (as compared to a control Rubus devoid of the same mutation) may be used with this invention. In some embodiments, the mutation in the endogenous gene may produce a protein that is reduced in functionality may be also used to produce Rubus plants or parts thereof of this invention having a compact growth habit and at least one improved agronomic trait as described herein as compared to a control Rubus plant.

In some embodiments, the at least one mutation in an endogenous gene as described herein may be a null allele (e.g., produces a non-functional protein or no protein). In some embodiments, the at least one mutation in an endogenous gene as described herein may be a dominant negative mutation (e.g., produces a protein having aberrant function that interferes with the function wild type gene product). In some embodiments, the at least one mutation in an endogenous gene as described herein in a Rubus plant may be a substitution, a deletion and/or an insertion. In some embodiments, the at least one mutation in an endogenous gene as described herein in a Rubus plant may be a substitution, a deletion and/or an insertion that results in a null allele, semi-dominant allele, weak loss of function allele, a null allele, or a hypomorphic mutation and a Rubus plant exhibiting at least one improved agronomic trait as described herein (e.g., a phenotype of reduced time to flowering, a longer duration of flowering, a more determinate flowering pattern, a more compact growth habit (e.g., increased apical dominance (increased determinacy), a reduced height, reduced/shortened stem length, reduced cane length, increased number of canes, reduced/shortened internode length, reduced lateral branching), improved yield characteristics, and the like). In some embodiments, the at least one mutation in an endogenous gene as described herein in a Rubus plant may be a substitution, a deletion and/or an insertion that results in a dominant negative mutation, a semi-dominant mutation, a weak loss of function mutation, a null mutation, or a hypomorphic mutation and a Rubus plant exhibiting a compact growth habit and optionally comprising at least one improved agronomic trait as described herein as compared to a control Rubus plant. For example, the mutation may be a substitution, a deletion and/or an insertion of one or more amino acid residues (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more consecutive amino acid residues, or more amino acids of the transcription factor) or the mutation may be a substitution, a deletion and/or an insertion of at least 5 consecutive nucleotides (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more consecutive nucleotides (e.g., up to the full length of the genomic sequence), or any range or value therein) (e.g., a base substitution, deletion and/or insertion) from the gene encoding the transcription factor. In some embodiments, the at least one mutation may be a base substitution to an A, a T, a G, or a C.

In some embodiments, any mutation in a TFL gene that produces a non-functional TFL polypeptide may be used to produce Rubus plants or parts thereof of this invention having a compact growth habit and resulting in at least one improved agronomic trait as described herein as compared to a control Rubus not comprising the same TFL gene mutation, optionally the at least one improved agronomic trait may be observed when the plants are planted at a higher density than the standard density. In some embodiments, the mutation in the TFL gene may produce a TFL protein that is reduced in functionality may be also used to produce Rubus plants or parts thereof of this invention having a compact growth habit and having at least one improved agronomic trait as described herein as compared to a control Rubus plant.

In some embodiments, the at least one mutation in an endogenous TFL gene may be a null allele (e.g., produces a non-functional protein or no protein). In some embodiments, the at least one mutation in an endogenous TFL gene may be a dominant negative mutation (e.g., produces a protein having aberrant function that interferes with the function of the wild type gene product). In some embodiments, the at least one mutation in an endogenous TFL gene in a Rubus plant may be a substitution, a deletion and/or an insertion. In some embodiments, the at least one mutation in an endogenous TFL gene in a Rubus plant may be a substitution, a deletion and/or an insertion that results in a null allele, semi-dominant allele, weak loss of function allele, a null allele, or a hypomorphic mutation and a Rubus plant exhibiting a phenotype of reduced time to flowering, a longer duration of flowering, a more determinate growth and flowering pattern (increased determinacy), a more compact growth habit (e.g., increased apical dominance, a reduced height, reduced/shortened stem length, reduced cane length, increased number of canes, reduced/shortened internode length, reduced lateral branching), and improved yield characteristics and the like. In some embodiments, the at least one mutation in an endogenous TFL gene in a Rubus plant may be a substitution, a deletion and/or an insertion that results in a dominant negative mutation, a semi-dominant mutation, a weak loss of function mutation, a null mutation, or a hypomorphic mutation and a Rubus plant exhibiting a compact growth habit and at least one improved agronomic trait as described herein as compared to a control Rubus plant. For example, the mutation may be a substitution, a deletion and/or an insertion of one or more amino acid residues (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more consecutive amino acid residues, or more amino acids of the transcription factor) or the mutation may be a substitution, a deletion and/or an insertion of at least 5 consecutive nucleotides (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or more consecutive nucleotides (e.g., up to the full length of the TFL genomic sequence), or any range or value therein) (e.g., a base substitution, deletion and/or insertion) from the gene encoding the transcription factor. In some embodiments, the at least one mutation may be a base substitution to an A, a T, a G, or a C.

In some embodiments, a mutation in an endogenous gene as described herein or its encoded protein, which is produced by methods of this invention may be a deletion. In some embodiments, a deletion may result in a truncation of the encoded protein or a deletion of a portion or the entire encoded polypeptide. In some embodiments, the mutation may be an N-terminal truncation or a C-terminal truncation. In some embodiments, the deletion may be a within the polypeptide or may encompass the entire polypeptide. When the mutation results in a C-terminal truncation in the encoded protein, the C-terminal truncation may comprise a truncation of at least 1 amino acid residue.

A mutation in an endogenous gene as described herein provides Rubus plants that exhibit a compact growth habit and have at least one improved agronomic trait as described herein as compared to a control Rubus may be a null allele (optionally, when the plants are planted at a higher density than the standard density as described herein). In some embodiments, a mutation in the endogenous gene that provides Rubus plants having a compact growth habit and at least one improved agronomic trait as described herein as compared to a control Rubus may be dominant negative mutation, a semi-dominant mutation, weak loss of function mutation, a null mutation, or a hypomorphic mutation, optionally where the mutation may be a non-natural mutation.

In some embodiments, a mutation in a TFL protein/gene produced by methods of this invention may be a deletion. In some embodiments, a deletion may result in a truncation of the TFL protein or a deletion of a portion or the entire TFL polypeptide. In some embodiments, the mutation may be an N-terminal truncation or a C-terminal truncation. In some embodiments, the deletion may be a within the polypeptide or may encompass the entire polypeptide. When the mutation results in a C-terminal truncation in the TFL protein, the C-terminal truncation may comprise a truncation of at least 1 amino acid residue (e.g., about 1, about 5, about 10, about 15, about 20, about 30, about 40 or about 50 amino acid residues to about 60, about 70, about 80, about 90, about 100, about 110, about 120, about 130, about 140, about 150, about 160, about 170, or about 172 consecutive amino acid residues or more) (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 171, or 172 consecutive amino acid residues, or more, or any range or value therein) from the C- or N-terminus of the TFL protein (e.g., SEQ ID NOs:74, 108, 116, 128, 142, 154, 164, 174, 186, 216, 236 or 253). In some embodiments, the polynucleotide encoding a truncated TFL polypeptide may comprise a deletion of at least 3 consecutive base pairs (e.g., about 3, 4, 5, 6, 9, 12, 15, 18, 21, 24, 27, 30, 33, 36, 39, 40, 50, 100 consecutive base pairs to about 150, 200, 250, 300, 350, 400, 450, 500, 510, 515, 516, 517, 518, 519, 520, 525, 550, 600, 700, 800, 900, or more consecutive base pairs; e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 130, 140, 150, 175, 200, 225, 250, 300, 350, 400, 450, 500, 505, 510, 515, 516, 517, 518, 519, 520, 525,550, 600, 650, 700, 750, 800, 850, 900, or 950 or more consecutive base pairs, or any range or value therein) from an endogenous gene encoding the TFL polypeptide (e.g., SEQ ID NOs:72, 73, 106, 107, 114, 115, 126, 127, 140, 141, 153, 162, 163, 172, 173, 184, 185, 214, 215, 234, 235, or 252). In some embodiments, a mutated endogenous TFL gene may be a non-natural mutation.

A mutation in an endogenous gene encoding a TFL protein that provides Rubus plants that exhibit a compact growth habit and having at least one improved agronomic trait as described herein as compared to a control Rubus may be a null allele. In some embodiments, a mutation in an endogenous gene encoding a TFL protein that provides Rubus plants having compact growth and at least one improved agronomic trait as described herein as compared to a control Rubus may be dominant negative mutation, a semi-dominant mutation, weak loss of function mutation, a null mutation, or a hypomorphic mutation, optionally where the mutation may be a non-natural mutation.

In some embodiments, a mutation in an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof) may be made following cleavage by an editing system that comprises a nuclease and a DNA-binding domain that binds to a target site within the endogenous gene. In some embodiments, a mutation in an endogenous TFL gene may be made following cleavage by an editing system that comprises a nuclease and a DNA-binding domain that binds to a target site within a target nucleic acid comprising a nucleotide sequence having at least 80% sequence identity to any one of the nucleotide sequence of SEQ ID NOs:72, 73, 106, 107, 114, 115, 126, 127, 140, 141, 153, 162, 163, 172, 173, 184, 185, 214, 215, 234, 235, or 252, or encoding a polypeptide comprising the sequence of any one of the amino acid sequences of SEQ ID NOs:74, 108, 116, 128, 142, 154, 164, 174, 186, 216, 236 or 253. In some embodiments, the nuclease cleaves the endogenous TFL gene, and a mutation is introduced into the endogenous TFL gene.

Guide nucleic acids (e.g., gRNA, gDNA, crRNA, crDNA) may be designed to target the endogenous genes for modification/editing as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof). In some embodiments, guide nucleic acids are provided that bind to a target site in a TFL gene, wherein the endogenous TFL gene: (a) comprises a nucleotide sequence having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, (b) comprises a region having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270, (c) encodes an amino acid sequence having at least 80% sequence identity to any one of SEQ ID NOs:74, 108, 116, 128, 142, 154, 164, 174, 186, 216, 236 or 253, and/or (d) encodes a region having at least 80% sequence identity to any one of the amino acid sequences of SEQ ID NOs:193-197, 247, 248, or 271-276.

Additionally provided are guide nucleic acids that binds to a target site in a TFL gene, wherein the target site is in a region of the TFL gene having at least 80% sequence identity to any one of SEQ ID NOs:75-105, 109-113, 117-125, 129-139, 143-152, 155-161, 165-171, 175-183, 187-192, 217-233, 237-246, or 254-270. In some embodiment, a guide nucleic acid comprises a spacer having at least 80% sequence identity to the nucleotide sequence of any one of SEQ ID NOs:198-210, 211-213 or 249-251 optionally comprising the nucleotide sequence of any one of SEQ ID NOs:198-210, 211-213 or 249-251.

As used herein, “a CRISPR-Cas effector protein in association with a guide nucleic acid” refers to the complex that is formed between a CRISPR-Cas effector protein and a guide nucleic acid in order to direct the CRISPR-Cas effector protein to a target site in a gene.

In some embodiments, expression cassettes are provided that comprise (a) a polynucleotide encoding CRISPR-Cas effector protein comprising a cleavage domain and (b) a guide nucleic acid that binds to a target site in an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or an orthologue thereof), wherein the guide nucleic acid comprises a spacer sequence that is complementary to and binds to a portion of the endogenous gene.

In some embodiments, expression cassettes are provided that comprise (a) a polynucleotide encoding CRISPR-Cas effector protein comprising a cleavage domain and (b) a guide nucleic acid that binds to a target site in an endogenous TFL gene, wherein the guide nucleic acid comprises a spacer sequence that is complementary to and binds: (i) a portion of nucleic acid having at least 80% sequence identity to any one of the nucleotide sequences of SEQ ID NOs:72, 73, 106, 107, 114, 115, 126, 127, 140, 141, 153, 162, 163, 172, 173, 184, 185, 214, 215, 234, 235, or 252; (ii) a portion of nucleic acid having at least 80% sequence identity to any one of the nucleotide sequences of SEQ ID NOs:75-105, 109-113, 117-125, 129-139, 143-152, 155-161, 165-171, 175-183, 187-192, 217-233, 237-246, or 254-270; (iii) a portion of a nucleic acid encoding an amino acid sequence having at least 80% sequence identity to any one of the amino acid sequences SEQ ID NOs:74, 108, 116, 128, 142, 154, 164, 174, 186, 216, 236 or 253; and/or (iv) a portion of a nucleic acid encoding an amino acid sequence having at least 80% sequence identity to any one of the amino acid sequences of SEQ ID NOs:193-197, 247, 248, or 271-276.

Also provided herein is an endogenous TFL gene having a mutation, wherein the endogenous TFL gene having the mutation comprises a nucleic acid sequence having a mutation as described herein, optionally a mutation resulting in a truncated TFL polypeptide or no detectable TFL polypeptide. In some embodiments, a mutation in a TFL1 gene may be a non-natural mutation. Further provided is a nucleic acid encoding a null allele of a TFL gene, wherein the null allele when present in a Rubus plant results in the ability of the Rubus plant to be planted at a high density resulting in at least one improved agronomic trait as described herein as compared to a control Rubus. Additionally provided are nucleic acids encoding a dominant negative mutation of a TFL gene, which when present in a Rubus plant results in the Rubus plant exhibiting a compact growth habit and the ability of to be planted at a high density resulting in at least one improved agronomic trait as described herein as compared to a control Rubus plant. Also provided herein, are nucleic acids encoding a semi-dominant mutation, a weak loss of function mutation, a null mutation, or a hypomorphic mutation, wherein the semi-dominant mutation, weak loss of function mutation, null mutation, or hypomorphic mutation of a TFL gene as described herein, which when present in a Rubus plant results in the plant having a compact growth habit and the ability of the Rubus plant to be planted at a high density resulting in at least one improved agronomic trait as described herein as compared to a control Rubus plant. In some embodiments, the mutated endogenous TFL gene comprises a non-natural mutation.

Nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids (e.g., endogenous TFL genes of Rubus plants or endogenous CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) and/or their expression.

Any Rubus plant comprising an endogenous gene as described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof) that when mutated as described herein results in the ability of the Rubus plant to be planted at a high density and having at least one improved agronomic trait as described herein as compared to a control Rubus plant may be modified (e.g., mutated, e.g., base edited, cleaved, nicked, etc.) as described herein (e.g., using the polypeptides, polynucleotides, RNPs, nucleic acid constructs, expression cassettes, and/or vectors of the invention) to provide ability of the Rubus plant to be planted at a high density and having at least one improved agronomic trait as described herein as compared to a control Rubus plant. For example, the invention may provide a Rubus plant having a more compact growth habit when compared to a Rubus plant that is devoid of the mutated endogenous gene.

In some embodiments, the invention may provide a Rubus plant having one or more improved agronomic characteristics, such as a compact growth habit and, for example, increased yield per acre as compared to a Rubus plant that is devoid of the mutated endogenous gene. In some embodiments, an improved agronomic characteristic may be an increase in fruit yield, optionally an increase of about 25% to about 450% in fruit yield per acre (e.g., about 25, 50, 75, 100, 125, 130, 135, 140, 140, 150, 160, 170, 180, 190, 200, 225, 250, 275, 300, 325, 350, 375, 400, 425, or 450% increase in fruit yield per acre) as compared to a control plant (e.g., an isogenic or WT plant devoid of the modification in an endogenous gene as described herein) or an increase in fruit yield per acre (e.g., lbs/acre) that is about 2 times to about 4 times greater than a control plant (e.g., an isogenic or WT plant devoid of the modification in an endogenous gene as described herein). In some embodiments, an improved agronomic characteristic or trait may be increased harvesting efficiency, increased Brix in fruits of the Rubus plants, increased Brix:acidity ratio in fruits of the Rubus plants, decreased water usage (e.g., a decrease in the amount of water used per amount of marketable fruit produced, decreased nitrogen usage (e.g., a decrease in the amount of nitrogen applied (e.g., amount of nitrogen applied per amount of marketable fruit harvested)), a reduction in the amount of unmarketable fruit produced, an increase in the amount of marketable fruit produced or an increase in the amount of marketable fruit harvested per pound of nitrogen applied as compared to a control and as described herein.

A Rubus plant and/or plant part that may be modified as described herein may be any Rubus species, variety and/or cultivar. In some embodiments, a Rubus plant or part thereof useful with this invention is a caneberry, optionally blackberry, black raspberry, or red raspberry. Non-limiting examples of Rubus spp. include blackberry, black raspberry or red raspberry, and the like. I n some embodiments, the Rubus plant is Rubus occidentalis L., Rubus pergratus Blanch., Rubus oklahomus L.H. Bailey Rubus originalis L.H. Bailey, Rubus ortivus (L.H. Bailey) L.H. Bailey, Rubus parcifrondifer L.H. Bailey, Rubus odoratus L., Rubus parvifolius L., Rubus pedatus Sm., or Rubus phoenicolasius Maxim

The term “plant part,” as used herein, includes but is not limited to reproductive tissues (e.g., petals, sepals, stamens, pistils, receptacles, anthers, pollen, flowers, fruits, flower bud, ovules, seeds, and embryos); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus. The term “stem” as used herein refers the above ground structural axis of the plant consisting of both nodes (e.g., leaves and flowers) and internodes (e.g., connecting material between nodes).

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. A “protoplast” is an isolated plant cell without a cell wall or with only parts of the cell wall. Thus, in some embodiments of the invention, a transgenic cell comprising a nucleic acid molecule and/or nucleotide sequence of the invention is a cell of any plant or plant part including, but not limited to, a root cell, a leaf cell, a tissue culture cell, a seed cell, a flower cell, a fruit cell, a pollen cell, and the like. In some aspects of the invention, the plant part can be a plant germplasm. In some aspects, a plant cell can be non-propagating plant cell that does not regenerate into a plant.

“Plant cell culture” means cultures of plant units such as, for example, protoplasts, cell culture cells, cells in plant tissues, pollen, pollen tubes, ovules, embryo sacs, zygotes and embryos at various stages of development.

As used herein, a “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

In some embodiments of the invention, a transgenic tissue culture or transgenic plant cell culture is provided, wherein the transgenic tissue or cell culture comprises a nucleic acid molecule/nucleotide sequence of the invention. In some embodiments, transgenes may be eliminated from a plant developed from the transgenic tissue or cell by breeding of the transgenic plant with a non-transgenic plant and selecting among the progeny for the plants comprising the desired gene edit and not the transgenes used in producing the edit.

An editing system that may be used to modify an endogenous gene as described herein of a Rubus plant to generate a Rubus plant, which can be planted at a high density as described herein, may be any site-specific (sequence-specific) genome editing system now known or later developed, which system can introduce mutations in a target specific manner. For example, an editing system (e.g., site- or sequence-specific editing system) can include, but is not limited to, a CRISPR-Cas editing system, a meganuclease editing system, a zinc finger nuclease (ZFN) editing system, a transcription activator-like effector nuclease (TALEN) editing system, a base editing system and/or a prime editing system, each of which can comprise one or more polypeptides and/or one or more polynucleotides that when expressed as a system in a cell can modify (mutate) a target nucleic acid in a sequence specific manner. In some embodiments, an editing system (e.g., site- or sequence-specific editing system) can comprise one or more polynucleotides and/or one or more polypeptides, including but not limited to a nucleic acid binding domain (DNA binding domain), a nuclease, and/or other polypeptide, and/or a polynucleotide.

In some embodiments, an editing system can comprise one or more sequence-specific nucleic acid binding domains (DNA binding domains) that can be from, for example, a polynucleotide-guided endonuclease, a CRISPR-Cas endonuclease (e.g., CRISPR-Cas effector protein), a zinc finger nuclease, a transcription activator-like effector nuclease (TALEN) and/or an Argonaute protein. In some embodiments, an editing system can comprise one or more cleavage domains (e.g., nucleases) including, but not limited to, an endonuclease (e.g., Fok1), a polynucleotide-guided endonuclease, a CRISPR-Cas endonuclease (e.g., CRISPR-Cas effector protein), a zinc finger nuclease, and/or a transcription activator-like effector nuclease (TALEN). In some embodiments, an editing system can comprise one or more polypeptides that include, but are not limited to, a deaminase (e.g., a cytosine deaminase, an adenine deaminase), a reverse transcriptase, a Dna2 polypeptide, and/or a 5′ flap endonuclease (FEN). In some embodiments, an editing system can comprise one or more polynucleotides, including, but is not limited to, a CRISPR array (CRISPR guide) nucleic acid, extended guide nucleic acid, and/or a reverse transcriptase template.

In some embodiments, a method of modifying or editing an endogenous gene as described herein (e.g., TFL, CEN, BRC1, AP1, FT GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX IPL PIF4/PIF5, PHYB, HFR1, BFT, or orthologues thereof) may comprise contacting a target nucleic acid (e.g., an endogenous gene as described herein) with a base-editing fusion protein (e.g., a sequence specific DNA binding protein (e.g., a CRISPR-Cas effector protein or domain) fused to a deaminase domain (e.g., an adenine deaminase and/or a cytosine deaminase)) and a guide nucleic acid, wherein the guide nucleic acid is capable of guiding/targeting the base editing fusion protein to the target nucleic acid, thereby editing a locus within the target nucleic acid. In some embodiments, a base editing fusion protein and guide nucleic acid may be comprised in one or more expression cassettes. In some embodiments, the target nucleic acid may be contacted with a base editing fusion protein and an expression cassette comprising a guide nucleic acid. In some embodiments, the sequence-specific nucleic acid binding fusion proteins and guides may be provided as ribonucleoproteins (RNPs). In some embodiments, a cell may be contacted with more than one base-editing fusion protein and/or one or more guide nucleic acids that may target one or more target nucleic acids in the cell.

In some embodiments, a method of modifying or editing a TFL gene or another endogenous gene as described herein may comprise contacting a target nucleic acid (e.g., an endogenous gene as described herein) with a sequence-specific nucleic acid binding fusion protein (e.g., a sequence-specific DNA binding protein (e.g., a CRISPR-Cas effector protein or domain) fused to a peptide tag, a deaminase fusion protein comprising a deaminase domain (e.g., an adenine deaminase and/or a cytosine deaminase) fused to an affinity polypeptide that is capable of binding to the peptide tag, and a guide nucleic acid, wherein the guide nucleic acid is capable of guiding/targeting the sequence-specific nucleic acid binding fusion protein to the target nucleic acid and the sequence-specific nucleic acid binding fusion protein is capable of recruiting the deaminase fusion protein to the target nucleic acid via the peptide tag-affinity polypeptide interaction, thereby editing a locus within the target nucleic acid. In some embodiments, the sequence-specific nucleic acid binding fusion protein may be fused to the affinity polypeptide that binds the peptide tag and the deaminase may be fused to the peptide tag, thereby recruiting the deaminase to the sequence-specific nucleic acid binding fusion protein and to the target nucleic acid. In some embodiments, the sequence-specific binding fusion protein, deaminase fusion protein, and guide nucleic acid may be comprised in one or more expression cassettes. In some embodiments, the target nucleic acid may be contacted with a sequence-specific binding fusion protein, deaminase fusion protein, and an expression cassette comprising a guide nucleic acid. In some embodiments, the sequence-specific nucleic acid binding fusion proteins, deaminase fusion proteins and guides may be provided as ribonucleoproteins (RNPs).

In some embodiments, templated editing may be used to modify an endogenous gene as described herein in a Rubus plant or part thereof. Templated editing includes, but is not limited to, PRIME editing and REDRAW editing. In some embodiments, unique enzymes may be used, such as SHARC (see e.g., PCT/US2024/018165). In general, templated editing comprises a Cas polypeptide, a reverse transcriptase, a guide RNA, a primer binding site and a reverse transcriptase template. In some embodiments, a reverse transcriptase may comprise DNA polymerase activity. These components and their exact make up can vary depending on the type of templated editing that is used. For example, a guide RNA may be an “extended” guide RNA that comprises an extended portion (in addition to a spacer) that comprises the primer binding site and an edit to be incorporated into the target nucleic acid (e.g., reverse transcriptase template). Methods for templated editing are known in the art, see for example, U.S. Pat. Nos. 11,926,834, 11,643,652, U.S. patent application Ser. No. 17/142,570, U.S. patent application Ser. No. 17/078,919 and PCT/US2024/018165.

As used herein, a “CRISPR-Cas effector protein” is a protein or polypeptide or domain thereof that cleaves or cuts a nucleic acid, binds a nucleic acid (e.g., a target nucleic acid and/or a guide nucleic acid), and/or that identifies, recognizes, or binds a guide nucleic acid as defined herein. In some embodiments, a CRISPR-Cas effector protein may be an enzyme (e.g., a nuclease, endonuclease, nickase, etc.) or portion thereof and/or may function as an enzyme. In some embodiments, a CRISPR-Cas effector protein refers to a CRISPR-Cas nuclease polypeptide or domain thereof that comprises nuclease activity or in which the nuclease activity has been reduced or eliminated, and/or comprises nickase activity or in which the nickase has been reduced or eliminated, and/or comprises single stranded DNA cleavage activity (ss DNAse activity) or in which the ss DNAse activity has been reduced or eliminated, and/or comprises self-processing RNAse activity or in which the self-processing RNAse activity has been reduced or eliminated. A CRISPR-Cas effector protein may bind to a target nucleic acid.

In some embodiments, a sequence-specific DNA binding domain may be a CRISPR-Cas effector protein. In some embodiments, a CRISPR-Cas effector protein may be from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a CRISPR-Cas effector protein of the invention may be from a Type II CRISPR-Cas system or a Type V CRISPR-Cas system. In some embodiments, a CRISPR-Cas effector protein may be Type II CRISPR-Cas effector protein, for example, a Cas9 effector protein. In some embodiments, a CRISPR-Cas effector protein may be Type V CRISPR-Cas effector protein, for example, a Cas12 effector protein.

In some embodiments, a CRISPR-Cas effector protein may include, but is not limited to, a Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5 nuclease, optionally wherein the CRISPR-Cas effector protein may be a Cas9, Cas12a (Cpf1), Cas12b, Cas12c (C2c3), Cas12d (CasY), Cas12e (CasX), Cas12g, Cas12h, Cas12i, C2c4, C2c5, C2c8, C2c9, C2c10, Cas14a, Cas14b, and/or Cas14c effector protein.

In some embodiments, a CRISPR-Cas effector protein useful with the invention may comprise a mutation in its nuclease active site (e.g., RuvC, HNH, e.g., RuvC site of a Cas12a nuclease domain; e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

A CRISPR Cas9 effector protein or CRISPR Cas9 effector domain useful with this invention may be any known or later identified Cas9 nuclease. In some embodiments, a CRISPR Cas9 polypeptide can be a Cas9 polypeptide from, for example, Streptococcus spp. (e.g., S. pyogenes, S. thermophilus), Lactobacillus spp., Bifidobacterium spp., Kandleria spp., Leuconostoc spp., Oenococcus spp., Pediococcus spp., Weissella spp., and/or Olsenella spp. Example Cas9 sequences include, but are not limited to, the amino acid sequences of SEQ ID NO:59 and SEQ ID NO:60 or the nucleotide sequences of any one of SEQ ID NOs:61-71.

In some embodiments, the CRISPR-Cas effector protein may be a Cas9 polypeptide derived from Streptococcus pyogenes and recognizes the PAM sequence motif NGG, NAG, NGA (Mali et al, Science 2013; 339(6121): 823-826). In some embodiments, the CRISPR-Cas effector protein may be a Cas9 polypeptide derived from Streptococcus thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the CRISPR-Cas effector protein may be a Cas9 polypeptide derived from Streptococcus mutans and recognizes the PAM sequence motif NGG and/or NAAR (R=A or G) (See, e.g., Deveau et al, J BACTERIOL 2008; 190(4): 1390-1400). In some embodiments, the CRISPR-Cas effector protein may be a Cas9 polypeptide derived from Streptococcus aureus and recognizes the PAM sequence motif NNGRR (R=A or G). In some embodiments, the CRISPR-Cas effector protein may be a Cas9 protein derived from S. aureus, which recognizes the PAM sequence motif N GRRT (R=A or G). In some embodiments, the CRISPR-Cas effector protein may be a Cas9 polypeptide derived from S. aureus, which recognizes the PAM sequence motif N GRRV (R=A or G). In some embodiments, the CRISPR-Cas effector protein may be a Cas9 polypeptide that is derived from Neisseria meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the CRISPR-Cas effector protein may be a Cas13a protein derived from Leptotrichia shahii, which recognizes a protospacer flanking sequence (PFS) (or RNA PAM (rPAM)) sequence motif of a single 3′ A, U, or C, which may be located within the target nucleic acid.

In some embodiments, the CRISPR-Cas effector protein may be derived from Cas12a, which is a Type V Clustered Regularly Interspaced Short Palindromic Repeats (CRISPR)-Cas nuclease see, e.g., SEQ ID NOs:1-20). Cas12a differs in several respects from the more well-known Type II CRISPR Cas9 nuclease. For example, Cas9 recognizes a G-rich protospacer-adjacent motif (PAM) that is 3′ to its guide RNA (gRNA, sgRNA, crRNA, crDNA, CRISPR array) binding site (protospacer, target nucleic acid, target DNA) (3′-NGG), while Cas12a recognizes a T-rich PAM that is located 5′ to the target nucleic acid (5′-TTN, 5′-TTTN. In fact, the orientations in which Cas9 and Cas12a bind their guide RNAs are very nearly reversed in relation to their N and C termini. Furthermore, Cas12a enzymes use a single guide RNA (gRNA, CRISPR array, crRNA) rather than the dual guide RNA (sgRNA (e.g., crRNA and tracrRNA)) found in natural Cas9 systems, and Cas12a processes its own gRNAs. Additionally, Cas12a nuclease activity produces staggered DNA double stranded breaks instead of blunt ends produced by Cas9 nuclease activity, and Cas12a relies on a single RuvC domain to cleave both DNA strands, whereas Cas9 utilizes an HNH domain and a RuvC domain for cleavage.

A CRISPR Cas12a effector protein/domain useful with this invention may be any known or later identified Cas12a polypeptide (previously known as Cpf1) (see, e.g., U.S. Pat. No. 9,790,490, which is incorporated by reference for its disclosures of Cpf1 (Cas12a) sequences). The term “Cas12a”, “Cas12a polypeptide” or “Cas12a domain” refers to an RNA-guided nuclease comprising a Cas12a polypeptide, or a fragment thereof, which comprises the guide nucleic acid binding domain of Cas12a and/or an active, inactive, or partially active DNA cleavage domain of Cas12a. In some embodiments, a Cas12a useful with the invention may comprise a mutation in the nuclease active site (e.g., RuvC site of the Cas12a domain). A Cas12a domain or Cas12a polypeptide having a mutation in its nuclease active site, and therefore, no longer comprising nuclease activity, is commonly referred to as deadCas12a (e.g., dCas12a). In some embodiments, a Cas12a domain or Cas12a polypeptide having a mutation in its nuclease active site may have impaired activity, e.g., may have nickase activity.

Any deaminase domain/polypeptide useful for base editing may be used with this invention. In some embodiments, the deaminase domain may be a cytosine deaminase domain or an adenine deaminase domain. A cytosine deaminase (or cytidine deaminase) useful with this invention may be any known or later identified cytosine deaminase from any organism (see, e.g., U.S. Pat. No. 10,167,457 and Thuronyi et al. Nat. Biotechnol. 37:1070-1079 (2019), each of which is incorporated by reference herein for its disclosure of cytosine deaminases). Cytosine deaminases can catalyze the hydrolytic deamination of cytidine or deoxycytidine to uridine or deoxyuridine, respectively. Thus, in some embodiments, a deaminase or deaminase domain useful with this invention may be a cytidine deaminase domain, catalyzing the hydrolytic deamination of cytosine to uracil. In some embodiments, a cytosine deaminase may be a variant of a naturally occurring cytosine deaminase, including but not limited to a primate (e.g., a human, monkey, chimpanzee, gorilla), a dog, a cow, a rat, or a mouse. Thus, in some embodiments, an cytosine deaminase useful with the invention may be about 70% to about 100% identical to a wild type cytosine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring cytosine deaminase).

In some embodiments, a cytosine deaminase useful with the invention may be an apolipoprotein B mRNA-editing complex (APOBEC) family deaminase. In some embodiments, the cytosine deaminase may be an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, an APOBEC4 deaminase, a human activation induced deaminase (hAID), an rAPOBEC1, FERNY, and/or a CDA1, optionally a pmCDA1, an atCDA1 (e.g., At2g19570), and evolved versions of the same (e.g., SEQ ID NO:27, SEQ ID NO:28 or SEQ ID NO:29). In some embodiments, the cytosine deaminase may be an APOBEC1 deaminase having the amino acid sequence of SEQ ID NO:23. In some embodiments, the cytosine deaminase may be an APOBEC3A deaminase having the amino acid sequence of SEQ ID NO:24. In some embodiments, the cytosine deaminase may be an CDA1 deaminase, optionally a CDA1 having the amino acid sequence of SEQ ID NO:25. In some embodiments, the cytosine deaminase may be a FERNY deaminase, optionally a FERNY having the amino acid sequence of SEQ ID NO:26. In some embodiments, a cytosine deaminase useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical) to the amino acid sequence of a naturally occurring cytosine deaminase (e.g., an evolved deaminase). In some embodiments, a cytosine deaminase useful with the invention may be about 70% to about 99.5% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identical) to the amino acid sequence of SEQ ID NO:23, SEQ ID NO:24, SEQ ID NO:25 or SEQ ID NO:26 (e.g., at least 80%, at least 85%, at least 90%, at least 92%, at least 95%, at least 96%, at least 97%, at least 98%, at least 99%, or at least 99.5% identical to the amino acid sequence of SEQ ID NO:23, SEQ ID NO:24, SEQ ID NO:25, SEQ ID NO:26, SEQ ID NO:27, SEQ ID NO:28 or SEQ ID NO:29). In some embodiments, a polynucleotide encoding a cytosine deaminase may be optimized for expression in a plant and the optimized polypeptide may be about 70% to 99.5% identical to the reference polynucleotide.

In some embodiments, a nucleic acid construct of this invention may further encode a uracil glycosylase inhibitor (UGI) (e.g., uracil-DNA glycosylase inhibitor) polypeptide/domain. Thus, in some embodiments, a nucleic acid construct encoding a CRISPR-Cas effector protein and a cytosine deaminase domain (e.g., encoding a fusion protein comprising a CRISPR-Cas effector protein domain fused to a cytosine deaminase domain, and/or a CRISPR-Cas effector protein domain fused to a peptide tag or to an affinity polypeptide capable of binding a peptide tag and/or a deaminase protein domain fused to a peptide tag or to an affinity polypeptide capable of binding a peptide tag) may further encode a uracil-DNA glycosylase inhibitor (UGI), optionally wherein the UGI may be optimized for expression in a plant. In some embodiments, the invention provides fusion proteins comprising a CRISPR-Cas effector polypeptide, a deaminase domain, and a UGI and/or one or more polynucleotides encoding the same, optionally wherein the one or more polynucleotides may be optimized for expression in a plant. In some embodiments, the invention provides fusion proteins, wherein a CRISPR-Cas effector polypeptide, a deaminase domain, and a UGI may be fused to any combination of peptide tags and affinity polypeptides as described herein, thereby recruiting the deaminase domain and UGI to the CRISPR-Cas effector polypeptide and a target nucleic acid. In some embodiments, a guide nucleic acid may be linked to a recruiting RNA motif and one or more of the deaminase domain and/or UGI may be fused to an affinity polypeptide that is capable of interacting with the recruiting RNA motif, thereby recruiting the deaminase domain and UGI to a target nucleic acid.

A “uracil glycosylase inhibitor” useful with the invention may be any protein that is capable of inhibiting a uracil-DNA glycosylase base-excision repair enzyme. In some embodiments, a UGI domain comprises a wild type UGI or a fragment thereof. In some embodiments, a UGI domain useful with the invention may be about 70% to about 100% identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% identical and any range or value therein) to the amino acid sequence of a naturally occurring UGI domain. In some embodiments, a UGI domain may comprise the amino acid sequence of SEQ ID NO:41 or a polypeptide having about 70% to about 99.5% sequence identity to the amino acid sequence of SEQ ID NO:41 (e.g., at least 80%, at least 85%, at least 90%, at least 92%, at least 95%, at least 96%, at least 97%, at least 98%, at least 99%, or at least 99.5% identical to the amino acid sequence of SEQ ID NO:41). For example, in some embodiments, a UGI domain may comprise a fragment of the amino acid sequence of SEQ ID NO:41 that is 100% identical to a portion of consecutive nucleotides (e.g., 10, 15, 20, 25, 30, 35, 40, 45, 50, 55, 60, 65, 70, 75, 80 consecutive nucleotides; e.g., about 10, 15, 20, 25, 30, 35, 40, 45, to about 50, 55, 60, 65, 70, 75, 80 consecutive nucleotides) of the amino acid sequence of SEQ ID NO:41. In some embodiments, a UGI domain may be a variant of a known UGI (e.g., SEQ ID NO:41) having about 70% to about 99.5% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%0, 90%, 91%, 92%, 93%, 94%, 95%, 96%, 97%, 98%, 99%, 99.5% sequence identity, and any range or value therein) to the known UGI. In some embodiments, a polynucleotide encoding a UGI may be optimized for expression in a plant (e.g., a plant) and the optimized polypeptide may be about 70% to about 99.5% identical to the reference polynucleotide.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a wild type E. coli TadA comprises the amino acid sequence of SEQ ID NO:30. In some embodiments, a mutated/evolved E. coli TadA* comprises the amino acid sequence of SEQ ID NOs:31-40 (e.g., SEQ ID NOs:31, 32, 33, 34, 35, 36, 37, 38, 39 or 40). In some embodiments, a polynucleotide encoding a TadA/TadA* may be optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention comprising a CRISPR-Cas effector protein or a fusion protein thereof may be used in combination with a guide RNA (gRNA, CRISPR array, CRISPR RNA, crRNA), designed to function with the encoded CRISPR-Cas effector protein or domain, to modify a target nucleic acid. A guide nucleic acid useful with this invention comprises at least one spacer sequence and at least one repeat sequence. The guide nucleic acid is capable of forming a complex with the CRISPR-Cas nuclease domain encoded and expressed by a nucleic acid construct of the invention and the spacer sequence is capable of hybridizing to a target nucleic acid, thereby guiding the complex (e.g., a CRISPR-Cas effector fusion protein (e.g., CRISPR-Cas effector domain fused to a deaminase domain and/or a CRISPR-Cas effector domain fused to a peptide tag or an affinity polypeptide to recruit a deaminase domain and optionally, a UGI) to the target nucleic acid, wherein the target nucleic acid may be modified (e.g., cleaved or edited) or modulated (e.g., modulating transcription) by the deaminase domain.

As an example, a nucleic acid construct encoding a Cas9 domain linked to a cytosine deaminase domain (e.g., fusion protein) may be used in combination with a Cas9 guide nucleic acid to modify a target nucleic acid, wherein the cytosine deaminase domain of the fusion protein deaminates a cytosine base in the target nucleic acid, thereby editing the target nucleic acid. In a further example, a nucleic acid construct encoding a Cas9 domain linked to an adenine deaminase domain (e.g., fusion protein) may be used in combination with a Cas9 guide nucleic acid to modify a target nucleic acid, wherein the adenine deaminase domain of the fusion protein deaminates an adenosine base in the target nucleic acid, thereby editing the target nucleic acid.

Likewise, a nucleic acid construct encoding a Cas12a domain (or other selected CRISPR-Cas nuclease, e.g., C2c1, C2c3, Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5) linked to a cytosine deaminase domain or adenine deaminase domain (e.g., fusion protein) may be used in combination with a Cas12a guide nucleic acid (or the guide nucleic acid for the other selected CRISPR-Cas nuclease) to modify a target nucleic acid, wherein the cytosine deaminase domain or adenine deaminase domain of the fusion protein deaminates a cytosine base in the target nucleic acid, thereby editing the target nucleic acid.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof, a repeat of a Type V C2cl CRISPR Cas system, or a fragment thereof, a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer.

A “repeat sequence” as used herein, refers to, for example, any repeat sequence of a wild-type CRISPR Cas locus (e.g., a Cas9 locus, a Cas12a locus, a C2cl locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. Nucleic Acids Res. 35(Web Server issue):W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to a target nucleic acid (e.g., target DNA) (e.g., protospacer) (e.g., a portion of consecutive nucleotides of a sequence of any one of the endogenous Rubus genes described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, BFT, or orthologues thereof). For example, a spacer sequence for use in editing an endogenous TFL gene may be complementary to a portion of a sequence (a) having at least 80% sequence identity to any one of SEQ ID NOs:72, 73, 106, 107, 114, 115, 126, 127, 140, 141, 153, 162, 163, 172, 173, 184, 185, 214, 215, 234, 235, or 252, or SEQ ID NOs:75-105, 109-113, 117-125, 129-139, 143-152, 155-161, 165-171, 175-183, 187-192, 217-233, 237-246, or 254-270; and/or (b) encoding an amino acid sequence having at least 80% sequence identity to any one of SEQ ID NOs:74, 108, 116, 128, 142, 154, 164, 174, 186, 216, 236 or 253, or SEQ ID NOs:193-197, 247, 248, or 271-276). In some embodiments, a spacer sequence (e.g., one or more spacers) targeting an endogenous Rubus TFL gene may include, but is not limited to, the nucleotide sequences of any one of SEQ ID NOs:193-197, 247, 248, or 271-276, or any combination thereof. A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more (e.g., 99.1, 99.2, 99.3, 99.4, 99.5, 99.6, 99.7, 99.8, 99.9, or 100%)) to a target nucleic acid. Thus, in some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length.

In some embodiments, the 5′ region of a spacer sequence of a guide nucleic acid may be identical to a target DNA, while the 3′ region of the spacer may be substantially complementary to the target DNA (e.g., Type V CRISPR-Cas), or the 3′ region of a spacer sequence of a guide nucleic acid may be identical to a target DNA, while the 5′ region of the spacer may be substantially complementary to the target DNA (e.g., Type II CRISPR-Cas), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

As used herein, a “target nucleic acid”, “target DNA,” “target nucleotide sequence,” “target region,” or a “target region in the genome” refers to a region of a plant's genome that is fully complementary (100% complementary) or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a guide nucleic acid of this invention. A target region useful for a CRISPR-Cas system may be located immediately 3′ (e.g., Type V CRISPR-Cas system) or immediately 5′ (e.g., Type II CRISPR-Cas system) to a PAM sequence in the genome of the organism (e.g., a plant genome). A target region may be selected from any region of at least 15 consecutive nucleotides (e.g., 16, 17, 18, 19, 20, 21, 22, 23, 24, 25, 26, 27, 28, 29, 30 nucleotides, and the like) located immediately adjacent to a PAM sequence.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type V CRISPR-Cas systems, the PAM is located at the 5′ end on the non-target strand and at the 3′ end of the target strand (see below, as an example).

5′-NNNNNNNNNNNNNNNNNNN-3′ RNA Spacer
||||||||||||||||||||
3′AAANNNNNNNNNNNNNNNNNNN-5′ Target strand)
||||
5′TTTNNNNNNNNNNNNNNNNNNN-3′ Non-target strand

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems (Nature Reviews Microbiology 13:722-736 (2015)). Guide structures and PAMs are described in by R. Barrangou (Genome Biol. 16:247 (2015)).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. 2013. Nat. Methods 10:1116-1121; Jiang et al. 2013. Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. 2014. Appl. Environ. Microbiol. 80:994-1001; Mojica et al. 2009. Microbiology 155:733-740).

In some embodiments, the present invention provides expression cassettes and/or vectors comprising the nucleic acid constructs of the invention (e.g., one or more components of an editing system of the invention). In some embodiments, expression cassettes and/or vectors comprising the nucleic acid constructs of the invention and/or one or more guide nucleic acids may be provided. In some embodiments, a nucleic acid construct of the invention encoding a base editor (e.g., a construct comprising a CRISPR-Cas effector protein and a deaminase domain (e.g., a fusion protein)) or the components for base editing (e.g., a CRISPR-Cas effector protein fused to a peptide tag or an affinity polypeptide, a deaminase domain fused to a peptide tag or an affinity polypeptide, and/or a UGI fused to a peptide tag or an affinity polypeptide), may be comprised on the same or on a separate expression cassette or vector from that comprising the one or more guide nucleic acids. When the nucleic acid construct encoding a base editor or the components for base editing is/are comprised on separate expression cassette(s) or vector(s) from that comprising the guide nucleic acid, a target nucleic acid may be contacted with (e.g., provided with) the expression cassette(s) or vector(s) encoding the base editor or components for base editing in any order from one another and the guide nucleic acid, e.g., prior to, concurrently with, or after the expression cassette comprising the guide nucleic acid is provided (e.g., contacted with the target nucleic acid).

Fusion proteins of the invention may comprise sequence-specific nucleic acid binding domains (e.g., sequence-specific DNA binding domains), CRISPR-Cas polypeptides, and/or deaminase domains fused to peptide tags or affinity polypeptides that interact with the peptide tags, as known in the art, for use in recruiting the deaminase to the target nucleic acid. Methods of recruiting may also comprise guide nucleic acids linked to RNA recruiting motifs and deaminases fused to affinity polypeptides capable of interacting with RNA recruiting motifs, thereby recruiting the deaminase to the target nucleic acid. Alternatively, chemical interactions may be used to recruit polypeptides (e.g., deaminases) to a target nucleic acid.

A peptide tag (e.g., epitope) useful with this invention may include, but is not limited to, a GCN4 peptide tag (e.g., Sun-Tag), a c-Myc affinity tag, an HA affinity tag, a His affinity tag, an S affinity tag, a methionine-His affinity tag, an RGD-His affinity tag, a FLAG octapeptide, a strep tag or strep tag II, a V5 tag, and/or a VSV-G epitope. Any epitope that may be linked to a polypeptide and for which there is a corresponding affinity polypeptide that may be linked to another polypeptide may be used with this invention as a peptide tag. In some embodiments, a peptide tag may comprise 1 or 2 or more copies of a peptide tag (e.g., repeat unit, multimerized epitope (e.g., tandem repeats)) (e.g., 1, 2, 3, 4, 5, 6, 7, 8, 9, 10, 11, 12, 13, 14, 15, 16, 17, 18, 19, 20, 21, 22, 23, 24, 25 or more repeat units. In some embodiments, an affinity polypeptide that interacts with/binds to a peptide tag may be an antibody. In some embodiments, the antibody may be a scFv antibody. In some embodiments, an affinity polypeptide that binds to a peptide tag may be synthetic (e.g., evolved for affinity interaction) including, but not limited to, an affibody, an anticalin, a monobody and/or a DARPin (see, e.g., Sha et al., Protein Sci. 26(5):910-924 (2017)); Gilbreth (Curr Opin Struc Biol 22(4):413-420 (2013)), U.S. Pat. No. 9,982,053, each of which are incorporated by reference in their entireties for the teachings relevant to affibodies, anticalins, monobodies and/or DARPins. Example peptide tag sequences and their affinity polypeptides include, but are not limited to, the amino acid sequences of SEQ ID NOs:45-47.

In some embodiments, a guide nucleic acid may be linked to an RNA recruiting motif, and a polypeptide to be recruited (e.g., a deaminase) may be fused to an affinity polypeptide that binds to the RNA recruiting motif, wherein the guide binds to the target nucleic acid and the RNA recruiting motif binds to the affinity polypeptide, thereby recruiting the polypeptide to the guide and contacting the target nucleic acid with the polypeptide (e.g., deaminase). In some embodiments, two or more polypeptides may be recruited to a guide nucleic acid, thereby contacting the target nucleic acid with two or more polypeptides (e.g., deaminases). Example RNA recruiting motifs and their affinity polypeptides include, but are not limited to, the sequences of SEQ ID NOs:48-58.

In some embodiments, a polypeptide fused to an affinity polypeptide may be a reverse transcriptase and the guide nucleic acid may be an extended guide nucleic acid linked to an RNA recruiting motif. In some embodiments, an RNA recruiting motif may be located on the 3′ end of the extended portion of an extended guide nucleic acid (e.g., 5′-3′, repeat-spacer-extended portion (RT template-primer binding site)-RNA recruiting motif). In some embodiments, an RNA recruiting motif may be embedded in the extended portion.

In some embodiments of the invention, an extended guide RNA and/or guide RNA may be linked to one or to two or more RNA recruiting motifs (e.g., 1, 2, 3, 4, 5, 6, 7, 8, 9, 10 or more motifs; e.g., at least 10 to about 25 motifs), optionally wherein the two or more RNA recruiting motifs may be the same RNA recruiting motif or different RNA recruiting motifs. In some embodiments, an RNA recruiting motif and corresponding affinity polypeptide may include, but is not limited, to a telomerase Ku binding motif (e.g., Ku binding hairpin) and the corresponding affinity polypeptide Ku (e.g., Ku heterodimer), a telomerase Sm7 binding motif and the corresponding affinity polypeptide Sm7, an MS2 phage operator stem-loop and the corresponding affinity polypeptide MS2 Coat Protein (MCP), a PP7 phage operator stem-loop and the corresponding affinity polypeptide PP7 Coat Protein (PCP), an SfMu phage Com stem-loop and the corresponding affinity polypeptide Com RNA binding protein, a PUF binding site (PBS) and the affinity polypeptide Pumilio/fem-3 mRNA binding factor (PUF), and/or a synthetic RNA-aptamer and the aptamer ligand as the corresponding affinity polypeptide. In some embodiments, the RNA recruiting motif and corresponding affinity polypeptide may be an MS2 phage operator stem-loop and the affinity polypeptide MS2 Coat Protein (MCP). In some embodiments, the RNA recruiting motif and corresponding affinity polypeptide may be a PUF binding site (PBS) and the affinity polypeptide Pumilio/fem-3 mRNA binding factor (PUF).

In some embodiments, the components for recruiting polypeptides and nucleic acids may those that function through chemical interactions that may include, but are not limited to, rapamycin-inducible dimerization of FRB-FKBP; Biotin-streptavidin; SNAP tag; Halo tag; CLIP tag; DmrA-DmrC heterodimer induced by a compound; bifunctional ligand (e.g., fusion of two protein-binding chemicals together, e.g., dihyrofolate reductase (DHFR).

In some embodiments, the nucleic acid constructs, expression cassettes or vectors of the invention that are optimized for expression in a plant may be about 70% to 100% identical (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100%) to the nucleic acid constructs, expression cassettes or vectors comprising the same polynucleotide(s) but which have not been optimized for expression in a plant (e.g., a Rubus plant).

Further provided herein are cells comprising one or more polynucleotides, guide nucleic acids, nucleic acid constructs, expression cassettes or vectors of the invention.

The nucleic acid constructs of the invention (e.g., a construct comprising a sequence specific DNA binding domain, a CRISPR-Cas effector domain, a deaminase domain, reverse transcriptase (RT), RT template and/or a guide nucleic acid, etc.) and expression cassettes/vectors comprising the same may be used as an editing system of this invention for modifying target nucleic acids and/or their expression.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### EXAMPLES

### Example 1. Growing and Trialing of Compact and Control Plants

Plugs for control and compact (edited) blackberry plants were vegetatively propagated before planting in the field. The plants were spaced at about 1.5 ft to about 2 ft apart with about 4 ft to about 5 ft 5 between rows. Plants were trellised in a commercial standard blackberry “V Trellis”. The marketable yields from the trial are collected at the plot level, and the yield per plant calculated by dividing by number of plants per plot.

A second harvest is provided after the plants are cutback completely to the crown of the plant and the plants then regrown Fruit is harvested bi-weekly by plot and sorted by marketable and unmarketable fruit.

Yield from the trials are collected at the plot level, and the yield divided by number of plants per plot to provide yield per plant. The yield per plant is multiplied by the number of plants per acre to provide overall or total yield (crates per acre and/or pounds per acre).

### Example 2. Identification of Endogenous Genes of Interest for Conferring Compactness to Rubus when Modified

Orthologs, paralogs, or functional homologs of genes described herein (e.g., TFL, CEN, BRC1, AP1, FT, GA20ox/GA3ox, GA2ox, GID1, DELLA, SLY1/GID2, YUCCA, PIN1/PIN3/PIN4, AUX1, BR1/BR2, AUX/IAA, DWF4/CPD, BRI1/BAK1, BIN2, CCD7/CCD8/D27/MAX, D14/MAX2, CKX, IPT, PIF4/PIF5, PHYB, HFR1, or BFT) are identified in Rubus species (e.g., Rubus argutus, Rubus fruticosus, Rubus idaeus, Rubus occidentalis) or in other fruit-bearing members of the family Rosaceae using standard molecular biology and bioinformatics techniques known to those of ordinary skill in the art.

Identification of these sequences is carried outby comparing one or more amino acid or nucleic acid sequences of a reference gene (e.g., a TFL, CEN, GA2ox, DELLA, BRC1, or YUCCA gene from maize, rice, or Arabidopsis thaliana) to a genomic or transcriptomic dataset from Rubus using sequence alignment tools such as BLASTP, TBLASTN, or BLASTN, or equivalent alignment algorithms. Searches employ typical parameters such as a BLOSUM62 substitution matrix and an expectation threshold of approximately 1e-10 or lower, though other settings of comparable stringency may also be suitable.

Candidate sequences showing strong alignment to the reference genes and conservation of key motifs are selected for further study. Sequence comparisons emphasize conservation across known functional domains rather than overall length.

Orthology is additionally confirmed by reciprocal best-hit analysis, wherein the Rubus candidate sequence, when used as a query against the source organism's proteome, retrieves the original reference gene as the best-scoring hit. Phylogenetic analysis may also be performed using maximum likelihood or Bayesian methods to demonstrate clustering of the Rubus sequence with known TFL/CEN or pathway-specific homologs within the corresponding clade.

Conservation of diagnostic motifs or catalytic domains characteristic of a given gene family is used to support orthology or functional equivalence. Examples include, but are not limited to: the PEBP domain (e.g., GNHD motif) of TFL/CEN family members; the DELLA and VHYNP motifs of growth-repressing DELLA proteins; the conserved HxD and GA recognition sites in GA20ox or GA3ox dioxygenases; the GHWT and SLY1-like domains of F-box proteins; the TCP domain of BRC1 transcription factors; and the YUCCA family FMO-like catalytic domain responsible for indole-3-acetic acid biosynthesis. Such motifs may exhibit conservation of at least 80% identity relative to the reference sequence within the aligned motif region.

An orthologous or functionally equivalent Rubus sequence may be identified by hybridization to a nucleic acid probe corresponding to a reference gene under stringent hybridization conditions (e.g., 0.1×SSC at 65° C.). Such hybridizing sequences, as well as allelic variants, splice variants, and isoforms thereof, are considered within the scope of “a gene encoding a TFL/CEN polypeptide or a functional homolog thereof” as used herein.

Syntenic analysis may also be employed to confirm orthology, particularly in cases of gene family expansion or duplication. For such an analysis, a candidate Rubus sequence is considered an ortholog if it resides in a chromosomal region colinear with the locus of the reference gene in a model species, and is flanked by one or more conserved neighboring genes. For example, a Rubus gene flanked by FT or AP1-like loci in a region corresponding to the TFL1/CEN neighborhood of Arabidopsis or Antirrhinum may be considered a bona fide ortholog.

Functional equivalence is assessed by expression profiling, mutant complementation, or phenotypic correlation. For example, expression of the candidate gene in a tissue corresponding to that of the reference gene (e.g., shoot apical meristem, axillary bud, stem elongation zone) provides evidence of functional conservation. Complementation of an Arabidopsis tfl1 or cen mutant by a Rubus candidate gene, or conversely, production of a compact or determinate phenotype in Rubus upon loss or modification of the candidate gene, likewise indicates functional equivalence.

In addition, expression analysis may be conducted using qRT-PCR, RNA-seq, or in situ hybridization to confirm developmental or tissue-specific expression patterns. In some embodiments, sequence alignments and phylogenetic trees may be generated using software such as MEGA, Clustal Omega, MAFFT, or PhyML, employing default parameters or adjusted gap opening penalties to achieve optimal alignment quality.

Once identified, Rubus orthologs may be modified to alter their expression or activity using any gene editing or transgenic approach known in the art, including but not limited to: (i) targeted mutagenesis using CRISPR-Cas nucleases, TALENs, or zinc-finger nucleases; (ii) base editing or prime editing to introduce point mutations affecting protein function; (iii) RNA interference, antisense suppression, or artificial microRNA to reduce transcript abundance; and (iv) promoter replacement or enhancer modification to achieve upregulation or ectopic expression. Resulting plants exhibiting compact, semi-dwarf, or determinate growth phenotypes are encompassed within the scope of the present invention.

Accordingly, the invention encompasses not only specific genes exemplified herein, but also any Rubus nucleic acid or protein sequence sharing sufficient structural or functional identity with a reference plant architecture gene to confer, when modified, a compact growth phenotype suitable for high-density cultivation as described herein.

### Example 3. Results from Modifying Endogenous TFL Genes in Rubus

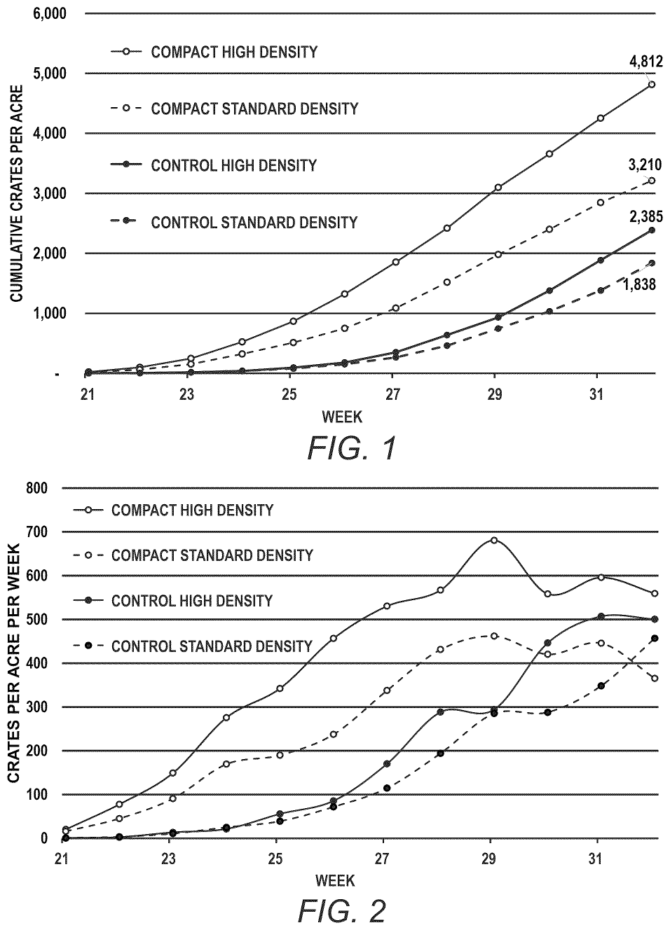

As shown in FIG. 1, yield in cumulative crates per acre per week (after planting) for Rubus plants comprising a mutation in an endogenous TFL gene (compact plants) is increased as compared to the control plants grown under standard density conditions and high density conditions. FIG. 2 shows yield in crates per acre per week (after planting) of Rubus plants comprising a mutation in an endogenous TFL gene (compact plants) is increased as compared to control plants grown under standard density conditions and high density conditions.

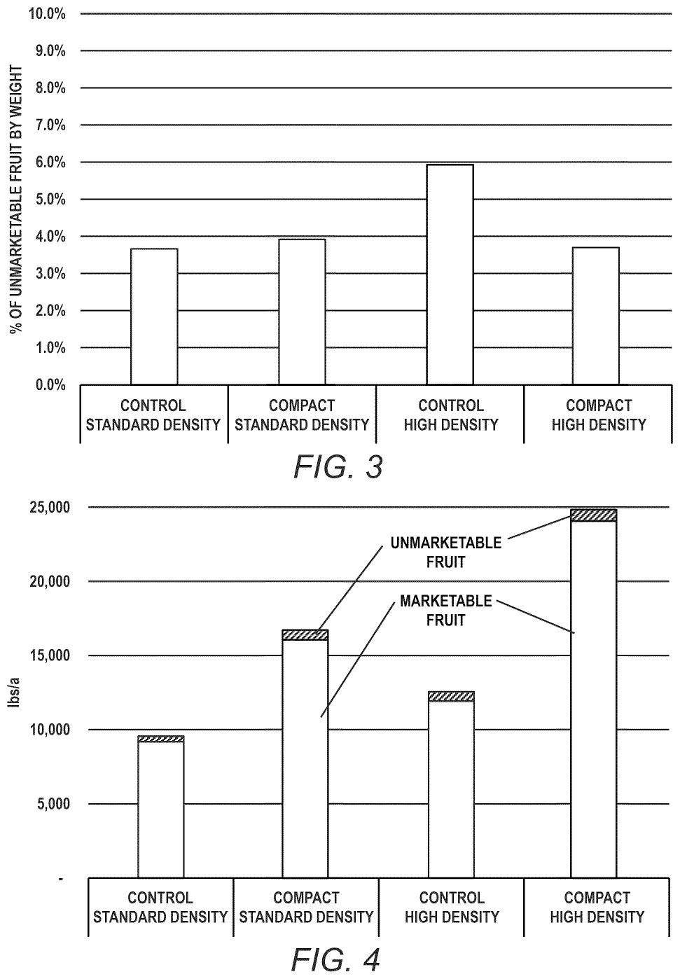

FIG. 3 shows that the percent of unmarketable fruit by weight for Rubus plants comprising a mutation in an endogenous TFL gene is less than the control plants grown under standard density conditions and high density conditions. FIG. 4 shows that the Rubus plants comprising a mutation in an endogenous TFL gene (compact plants) have increased marketable and decreased unmarketable fruit (pounds per acre) as compared to control plants grown under both standard density conditions and high density conditions.

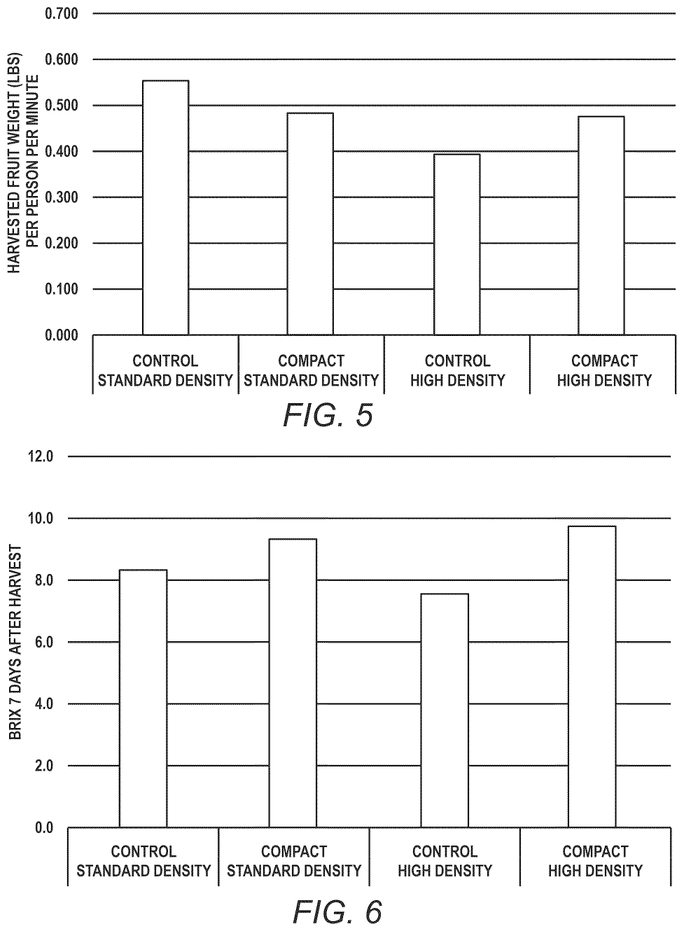

FIG. 5 shows an increase in harvesting efficiency (as measured by harvested fruit weight (lbs) per person per minute) for Rubus plants comprising a mutation in an endogenous TFL gene (compact plants) as compared to control plants grown under high density conditions.

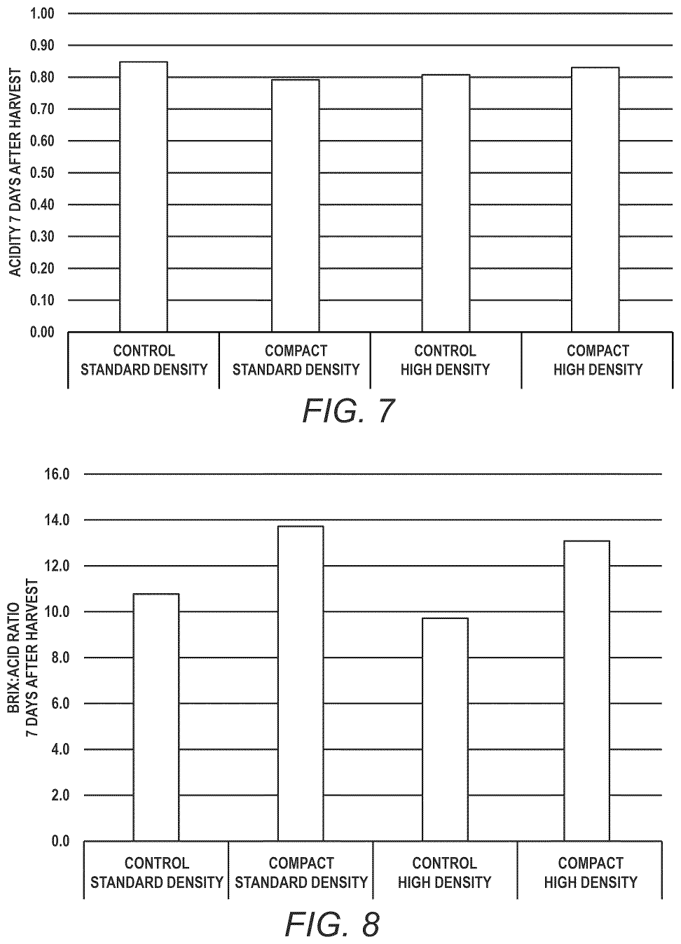

Brix was measured seven days after harvest for the fruit Rubus plants comprising a mutation in an endogenous TFL gene (compact plants) and the control plants. FIG. 6 shows that the Brix for the compact plants (comprising a mutation in an endogenous TFL gene) was increased over that of the control plants grown under both standard density conditions and high density conditions. In addition, the acidity was measured for the fruit of both the Rubus plants comprising a mutation in an endogenous TFL gene (compact plants) and control plants grown under standard density conditions and high density conditions. FIG. 7 shows that acidity was similar for the compact plants (comprising a mutation in an endogenous TFL gene) and the control plants grown under either standard density conditions of high density conditions. The Brix:acidity ratio was measured seven days after harvest for the fruit Rubus plants comprising a mutation in an endogenous TFL gene (compact plants) and control plants grown under standard density conditions and high density conditions. FIG. 8 shows that the Brix:acidity ratio was increased for the compact plants as compared to the control plants under both standard density conditions and high density conditions.

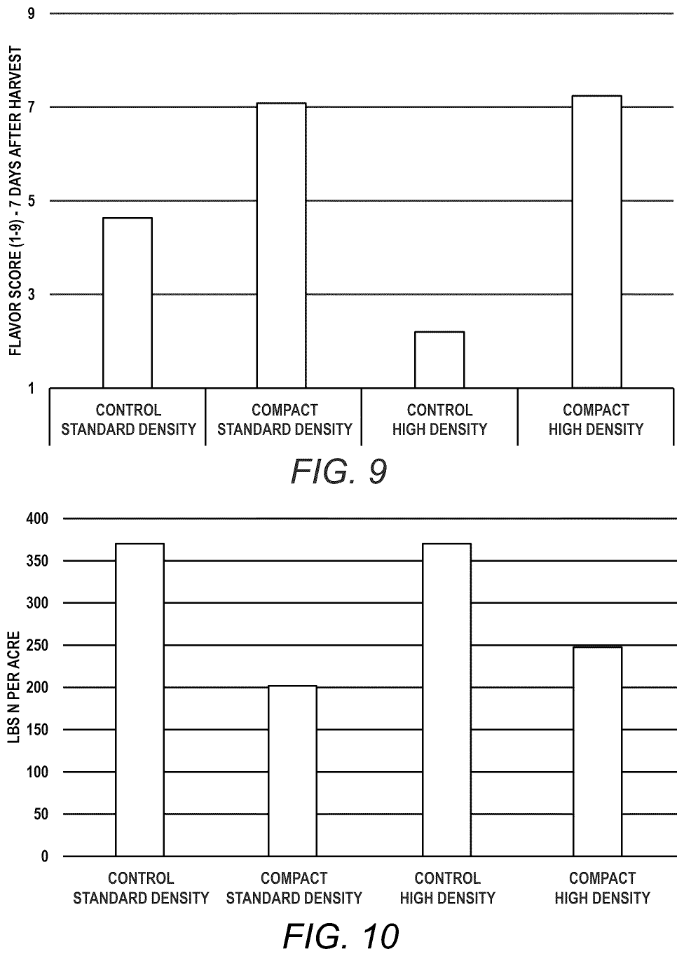

FIG. 9 provides the flavor score (score of 1-9) seven days after harvest for the fruit Rubus plants comprising a mutation in an endogenous TFL gene (compact plants) and control plants grown under standard density conditions and high density conditions and shows that the compact plants were given a higher flavor score than the control plants under both conditions.

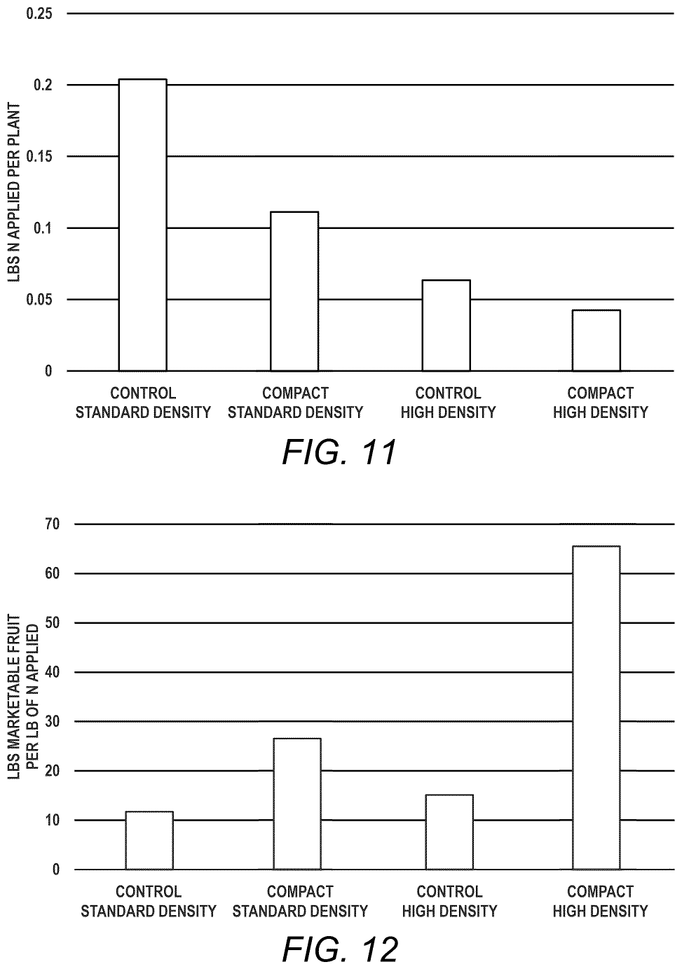

The Rubus plants comprising a mutation in an endogenous TFL gene (compact plants) also showed a reduction in the amount of nitrogen needed to grow the plants and produce fruit per acre (FIG. 10) and per plant (FIG. 11) as compared to control plants grown under standard density conditions and high density conditions. In addition, the pounds of marketable fruit harvested per pound of nitrogen applied for Rubus plants comprising a mutation in an endogenous TFL gene (compact plants) as compared to the control plants when grown under both conditions but particularly when grown under high density conditions (FIG. 12).

### Example 4. Additional Results from Modifying Endogenous TFL Genes in Rubus

In further study, the plant architecture of blackberry events having an edited endogenous TFL gene was compared to unedited control plants during a growout in a greenhouse setting. At the end of the season, plants were destructively harvested in order to compare size and number of canes. We observed that plant height (distance from soil line to highest point on plant) was reduced in all edited plants relative to the unedited control. We observed a similar trend when evaluating average cane length. For this measurement, we recorded lengths of all canes from all plants within each plot. Again plants with edited TFL genes exhibited a shorter average cane length and terminated in a flower more rapidly (increased determinacy) compared to the unedited control. In some cases, the canes of the edited plants exhibited a greater than 50% reduction in length when compared to the unedited control. Further, the average number of canes (shoots emerging from below the soil line or within 5 cm above soil line) for the edited plants was increased over the unedited control. These observations highlight the increased determinacy and propensity for branching in the TFL edited plants.

Overall, the Rubus plants of the present invention having at least one mutation in an endogenous TERMINAL FLOWER (TFL) gene encoding a TFL polypeptide and exhibiting a compact growth habit are shown to provide significant agronomic advantages over control Rubus plants not comprising the at least one mutation in an endogenous TFL gene encoding a TFL polypeptide and not exhibiting a compact growth habit, including, but not limited to, an increased yield per acre, increased harvesting efficiency, fruit with increased Brix and increased Brix:acidity ratio, increased flavor score, an increase in the amount of marketable fruit produced, an increased amount of marketable fruit per pound of nitrogen applied, and a decrease in water and nitrogen usage. The Rubus plants of the present invention also showed reduced height, reduced cane length, and increased cane numbers as compared to control Rubus plant not having at least one mutation in an endogenous TERMINAL FLOWER (TFL) gene encoding a TFL polypeptide and not exhibiting a compact growth habit.

The foregoing is illustrative of the present invention and is not to be construed as limiting thereof. The invention is defined by the following claims, with equivalents of the claims to be included therein.

What is claimed is:

1. A method of cultivating Rubus plants to improve agronomic performance, comprising planting Rubus plants at a high density, wherein the Rubus plants comprise at least one mutation in one or more endogenous genes associated with plant architecture and exhibit a compact growth habit resulting in an increased in yield per acre and improved agronomic efficiency as compared to control Rubus plants.

2. The method of claim 1, wherein improved agronomic efficiency comprises increased yield per acre, increased harvesting efficiency, increased Brix in fruits of the Rubus plants, increased Brix:acidity ratio in fruits of Rubus plants, decreased water usage (e.g., a decrease in the amount of water used per amount of marketable fruit produced, decreased nitrogen usage (e.g., a decrease in the amount of nitrogen applied (amount of nitrogen applied per amount of marketable fruit harvested)), increased amount of marketable fruit produced and a reduction in the amount of unmarketable fruit produced, an increase in the amount of marketable fruit produced and/or an increase in the amount of marketable fruit harvested per pound of nitrogen applied.

3. The method of claim 1, wherein planting the Rubus plants that comprise at least one mutation in one or more endogenous genes associated with plant architecture and exhibit a compact growth habit at a high density means planting at a density that is about 4000 plants per acre to about 7500 plants per acre, means planting at a density that is increased by about 100% to about 300% compared to planting of Rubus plants devoid of the at least one mutation in an endogenous gene and/or means planting at a density that is increased by about 2 times to 3 times compared to Rubus plants devoid of the at least one mutation in an endogenous gene.

4. The method of claim 1, wherein planting Rubus plants comprising at least one mutation in one or more endogenous genes associated with plant architecture and exhibiting compact growth habit at a high density means the Rubus plants are planted in rows that are about 3 foot to about 5.5 foot apart as compared to a row spacing of 6 ft to about 10 foot rows for Rubus plants devoid of the at least one mutation in an endogenous gene.

5. A crop production system comprising: (a) a population of Rubus plants having a compact growth habit and comprising at least one mutation in one or more endogenous genes that associated with plant architecture; and (b) the population of Rubus plants is planted at a density of at least 4,000 plants per acre, wherein the system provides improved yield per unit area and reduced resource usage per pound of marketable fruit relative to a system comprising control Rubus plants.

6. A method of improving at least one agronomic trait in a Rubus plant, comprising planting compact Rubus plants at a high density, wherein the compact Rubus plants comprise at least one mutation in one or more endogenous genes associated with plant architecture and exhibit increased apical dominance (increased determinacy), reduced height, reduced cane length, increased cane number, reduced lateral branching and/or reduced time to flowering, thereby resulting in at least one improved agronomic trait as compared to a control Rubus plant (devoid of the at least one mutation in the endogenous gene and does not exhibit compact growth).

7. The method of claim 1, wherein the one or more endogenous genes associated with plant architecture are TFL (TERMINAL FLOWER), CEN (CENTRORADIALIS), BRC1 (BRANCHED1), AP1 (APETALA1), FT (FLOWERING LOCUS T), GA20ox/GA3ox (GIBBERELLIN 20-OXIDASE, GIBBERELLIN 3-OXIDASE), GA2ox (GIBBERELLIN 2-OXIDASE), GID1 (GIBBERELLIN INSENSITIVE DWARF1), DELLA (DELLA PROTEIN (GA SIGNALING REPRESSOR), SLY1/GID2 (SLEEPY1, GIBBERELLIN INSENSITIVE DWARF2), YUCCA (YUCCA FLAVIN MONOOXYGENASE-LIKE), PIN1/PIN3/PIN4 (PIN-FORMED 1 AUXIN TRANSPORTER, PIN3, PIN4), AUX1, BR1/BR2 (BR1, BR2), AUX/IAA (AUX, IAA), DWF4/CPD (DWARF4 STEROL C-22 HYDROXYLASE, CPD), BRI1/BAK1 (BRASSINOSTEROID INSENSITIVE 1, BAK1), BIN2, CCD7/CCD8/D27/MAX (CCD7, CCD8, D27, MAX), D14/MAX2 (DWARF14 α/β HYDROLASE, MORE AXILLARY GROWTH 2), CKX (CYTOKININ OXIDASE/DEHYDROGENASE), IPT (ISOPENTENYL TRANSFERASE), PIF4/PIF5 (PIF4, PIF5), PHYB, HFR1 or BFT (BROTHER OF TFL AND FT), and/or orthologues thereof.

8. A method of improving at least one agronomic trait in a Rubus plant, comprising planting Rubus plants at a high density, wherein the Rubus plants comprise at least one mutation in an endogenous TERMINAL FLOWER (TFL) gene encoding a TFL polypeptide, and exhibit a compact growth habit resulting in an improvement of at least one agronomic trait as compared to a control Rubus plant (devoid of the at least one mutation in the TFL gene and does not exhibit compact growth).

9. The method of claim 8, wherein at least one agronomic trait comprises increased yield per acre, increased harvesting efficiency, increased Brix in fruits of the Rubus plants, increased Brix:acidity ratio in fruits of Rubus plants, decreased water usage (e.g., a decrease in the amount of water used per amount of marketable fruit produced, decreased nitrogen usage (e.g., a decrease in the amount of nitrogen applied (amount of nitrogen applied per amount of marketable fruit harvested)), increased amount of marketable fruit produced and a reduction in the amount of unmarketable fruit produced, an increase in the amount of marketable fruit produced or an increase in the amount of marketable fruit harvested per pound of nitrogen applied.

10. The method of claim 8, wherein planting the Rubus plants that comprise at least one mutation in an endogenous TERMINAL FLOWER (TFL) gene encoding a TFL polypeptide, and exhibit a compact growth habit at a high density means planting at a density that is about 4000 plants per acre to about 7500 plants per acre, means planting at a density that is increased by about 100% to about 300% compared to planting of Rubus plants devoid of the at least one mutation in an endogenous gene and/or means planting at a density that is increased by about 2 times to 3 times compared to Rubus plants devoid of the at least one mutation in an endogenous gene.

11. The method of claim 8, wherein planting Rubus plants comprising at least one mutation in an endogenous TFL gene encoding a TFL polypeptide and exhibiting compact growth habit at a high density means the Rubus plants are planted in rows that are about 3 foot to about 5.5 foot apart as compared to a row spacing of 6 ft to about 10 foot rows for Rubus plants devoid of the at least one mutation in an endogenous TFL gene.

12. The method of claim 9, wherein the yield per acre for the Rubus plants having the least one mutation in an endogenous TFL gene and exhibiting a compact growth habit is about 5000 crates/acre to about 10,000 crates/acre as compared to about 4000 crates/acre to about 4500 crates/acre for Rubus plants not comprising the at least one mutation in an endogenous TFL gene and planted under standard growing practices.

13. The method of claim 9, wherein the yield per acre for the Rubus plants having the least one mutation in an endogenous TFL gene and exhibiting a compact growth habit is about 20,000 pounds (lbs)/acre to about 50,000 lbs/acre, is about 30,000 pounds (lbs)/acre to about 90,000 lbs/acre, is about 25% to about 450% greater than that for Rubus plants that are devoid of the at least one mutation in an endogenous TFL gene and planted under standard growing practices, optionally wherein the yield per acre is about 100% to about 250% greater, or is about 2 times to about 4 times greater than that for Rubus plants that are devoid of the at least one mutation in an endogenous TFL gene and planted under standard growing practices.

14. The method of claim 9, wherein the harvesting efficiency (harvested fruit weight per person minute) is increased by about 20% to about 30% as compared to the control when grown under high density planting conditions, the Brix is increased by about 10% to about 35% as compared to the control, the Brix:acidity ratio is increased by about 10% to about 60% as compared to the control, water usage (gallons of water used per pound of fruit produced) is decreased by about 20% to about 90% as compared to the control, the amount of nitrogen applied per amount of marketable fruit produced is decreased by about 20% to about 80% as compared to the control, the amount of unmarketable fruit produced is reduced by at least about 1% to about 2.5% as compared to the control and/or the amount of marketable fruit produced is increased by at least about 100% to about 200% as compared to the control, and/or the amount of marketable fruit harvested per pound of nitrogen applied may be increased by about 20% to about 125%.

15. The method of claim 8, wherein the at least one mutation in an endogenous TFL gene results in a truncated TFL polypeptide and/or no detectable TFL polypeptide.

16. The method of claim 8, wherein the Rubus plant or part thereof comprises at least two endogenous TFL genes and at least one allele of the at least two endogenous TFL genes comprises the at least one mutation.

17. The method of claim 8, wherein the Rubus plant comprising at least one mutation in an endogenous TERMINAL FLOWER (TFL) gene (e.g., one or more TFL genes) encoding a TFL polypeptide, and exhibiting a compact growth habit when planted at a high density is more practicable commercially and agriculturally as compared to a control Rubus plant.

18. The method of claim 1, wherein the Rubus plants having the least one mutation in an endogenous gene associated with plant architecture do not require pruning or tipping.

19. The method of claim 8, wherein the Rubus plants having the least one mutation in the endogenous TFL gene and comprise a compact growth habit do not require pruning or tipping.

20. The method of claim 1, wherein the Rubus plant is a blackberry, raspberry, and/or black raspberry, optionally wherein the Rubus plant is Rubus occidentalis L., Rubus pergratus Blanch., Rubus oklahomus L.H. Bailey Rubus originalis L.H. Bailey, Rubus ortivus (L.H. Bailey) L.H. Bailey, Rubus parcifrondifer L.H. Bailey, Rubus odoratus L., Rubus parvifolius L., Rubus pedatus Sm., and Rubus phoenicolasius Maxim, optionally, wherein the Rubus plant is a blackberry plant.

21. The method of claim 8, wherein the Rubus plant is a blackberry, raspberry, and/or black raspberry, optionally wherein the Rubus plant is Rubus occidentalis L., Rubus pergratus Blanch., Rubus oklahomus L.H. Bailey Rubus originalis L.H. Bailey, Rubus ortivus (L.H. Bailey) L.H. Bailey, Rubus parcifrondifer L.H. Bailey, Rubus odoratus L., Rubus parvifolius L., Rubus pedatus Sm., and Rubus phoenicolasius Maxim, optionally, wherein the Rubus plant is a blackberry plant.


SEQUENCE LISTING (276 sequences)

SEQ ID NO: 1 - protein, 1228 aa, unidentified
  source 1..1228: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1241 aa, Butyrivibrio proteoclasticus
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEDARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 4 - protein, 1238 aa, Methanoplasma termitum
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDIVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIDRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLRPVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 5 - protein, 1281 aa, Eubacterium eligens
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLDKIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 6 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKVNYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 7 - protein, 1206 aa, unidentified
  source 1..1206: note = Lachnospiraceae sp.
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSVIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFDTQASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGLYLQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 8 - protein, 1233 aa, unidentified
  source 1..1233: note = Lachnospiraceae sp.
MDYGNGQFERRAPLTKTITLRLKPIGETRETIREQKLLEQDAAFRKLVETVTPIVDDCIRKIADNALCHFGTEYDFSCLGNAISKNDSKAIKKETEKVEKLLAKVLTENLPDGLRKVNDINSAAFIQDTLTSFVQDDADKRVLIQELKGKTVLMQRFLTTRITALTVWLPDRVFENFNIFIENAEKMRILLDSPLNEKIMKFDPDAEQYASLEFYGQCLSQKDIDSYNLIISGIYADDEVKNPGINEIVKEYNQQIRGDKDESPLPKLKKLHKQILMPVEKAFFVRVLSNDSDARSILEKILKDTEMLPSKIIEAMKEADAGDIAVYGSRLHELSHVIYGDHGKLSQIIYDKESKRISELMETLSPKERKESKKRLEGLEEHIRKSTYTFDELNRYAEKNVMAAYIAAVEESCAEIMRKEKDLRTLLSKEDVKIRGNRHNTLIVKNYFNAWTVFRNLIRILRRKSEAEIDSDFYDVLDDSVEVLSLTYKGENLCRSYITKKIGSDLKPEIATYGSALRPNSRWWSPGEKFNVKFHTIVRRDGRLYYFILPKGAKPVELEDMDGDIECLQMRKIPNPTIFLPKLVFKDPEAFFRDNPEADEFVFLSGMKAPVTITRETYEAYRYKLYTVGKLRDGEVSEEEYKRALLQVLTAYKEFLENRMIYADLNFGFKDLEEYKDSSEFIKQVETHNTFMCWAKVSSSQLDDLVKSGNGLLFEIWSERLESYYKYGNEKVLRGYEGVLLSILKDENLVSMRTLLNSRPMLVYRPKESSKPMVVHRDGSRVVDRFDKDGKYIPPEVHDELYRFFNNLLIKEKLGEKARKILDNKKVKVKVLESERVKWSKFYDEQFAVTFSVKKNADCLDTTKDLNAEVMEQYSESNRLILIRNTTDILYYLVLDKNGKVLKQRSLNIINDGARDVDWKERFRQVTKDRNEGYNEWDYSRTSNDLKEVYLNYALKEIAEAVIEYNAILIIEKMSNAFKDKYSFLDDVTFKGFETKKLAKLSDLHFRGIKDGEPCSFTNPLQLCQNDSNKILQDGVIFMVPNSMTRSLDPDTGFIFAINDHNIRTKKAKLNFLSKFDQLKVSSEGCLIMKYSGDSLPTHNTDNRVWNCCCNHPITNYDRETKKVEFIEEPVEELSRVLEENGIETDTELNKLNERENVPGKVVDAIYSLVLNYLRGTVSGVAGQRAVYYSPVTGKKYDISFIQAMNLNRKCDYYRIGSKERGEWTDFVAQLIN

SEQ ID NO: 9 - protein, 1227 aa, unidentified
  source 1..1227: note = Lachnospiraceae sp.
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIANINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIASNKEWLEYAQTSVKH

SEQ ID NO: 10 - protein, 1264 aa, Leptospira inadai
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYNKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNPLNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDISGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 11 - protein, 1373 aa, Moraxella bovoculi
MLFQDFTHLYPLSKTVRFELFIDRTLEHIHAKNFLSQDETMADMHQKVKVILDDYHRDFIADMMGEVKLTKLAEFYDVYLKFRKNPKDDELQKAQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFKDINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQKDFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIQASQAFFGKFDKICYNADKDYFEFHIDYAKFTDKAKNSRQIWTICSHGDKRYVYDKTANQNKGAAKGINVNDILKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 12 - protein, 1352 aa, unidentified
  source 1..1352: note = Parcubacteria bacterium
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITESKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKIKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 13 - protein, 1260 aa, Porphyromonas crevioricanis
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 14 - protein, 1324 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIDALKGFYIEDGFENNVLTPLATLLSSLDKYNLNGIFIRNNEALSSLSQNVYRNFSIDEAIDAQNAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNKDDLNLSISSTEWLDFVREKPYLK

SEQ ID NO: 15 - protein, 1484 aa, unidentified
  VARIANT 1073: note = misc_feature - Xaa can be any naturally occurring amino acid
  source 1..1484: note = Peregrinibacteria bacterium
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAMIKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTWIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDXWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKKKRPAATKK

SEQ ID NO: 16 - protein, 1245 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSISEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQHGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 17 - protein, 1250 aa, Smithella sp.
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYQWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 18 - DNA, 3987 bp, synthetic construct
  misc_feature 1..3987: note = Synthetic polynucleotide
  source 1..3987: mol_type = other DNA
atggccgggagcaagaagcgccggataaagcaggacacgcagttcgagggcttcaccaacctgtaccaagtctccaagacgctccggttcgagcttatcccgcaagggaagaccctgaaacacatccaggaacaaggtttcatcgaggaggacaaggcccgcaacgaccactacaaggagctcaagcccataatcgatcggatctacaagacgtacgccgaccagtgcctccaactggtgcagctcgactgggagaacctgagcgccgccattgacagctaccgcaaggaaaagacggaggagacgcgcaacgcccttattgaggagcaagccacctaccgcaacgccatccacgactacttcatcgggcgcaccgacaacctgacggacgcgatcaacaagcgccacgcggaaatctacaagggccttttcaaggccgagctcttcaacgggaaggtcctaaaacagctcgggactgtcacgacaaccgagcatgagaacgccctccttcgcagcttcgacaagttcaccacatacttctcgggcttctaccggaaccgcaagaacgttttcagcgccgaggacatctccaccgccatcccgcacaggatcgtccaggacaacttccccaagttcaaggagaactgccacatcttcacgcgcctgattacagccgtaccttcacttcgtgagcacttcgagaacgtcaaaaaggccatcgggatcttcgtctccacgtccatcgaggaggtattctctttcccgttctataaccagctcctgacccagacgcagatcgacctctacaaccagctactgggcggcatcagccgggaggccgggaccgagaaaataaagggcctcaacgaagttctcaacctggccatccagaagaacgacgagaccgcgcatatcatcgcatccctgccgcatcgcttcattcctttgttcaagcagatattgagcgaccggaacaccctctcgttcatcctcgaagaattcaagagcgacgaggaggtcattcagtctttctgcaagtacaagacgctcctacggaatgagaatgtgctggagaccgcggaggcactcttcaatgagctgaactccattgacctgacccacatcttcattagccacaagaaactggagacgatctccagcgccctgtgcgaccactgggacactctccgcaacgccctctacgaacgccggatctccgaacttaccggcaagataactaagtcggctaaggagaaggtgcaacggagcctcaagcacgaggacatcaaccttcaggaaatcatctcagccgcgggcaaggagctgagcgaggcgtttaagcagaaaacatcggagatactgagccacgcgcacgcggccctggatcaaccgctgccgacgactctcaagaagcaagaggagaaggaaatccttaagtcccagctcgactcgctgctcggcctctatcacttgctcgactggttcgcggttgatgagtccaacgaggtggacccggagttctccgcgcgcctcacgggtattaagctggagatggagccaagcttaagcttctacaacaaggcccgcaactacgcgaccaaaaaaccgtactcagtcgagaaattcaagctgaatttccagatgcctacattggcgagggggtgggacgtgaaccgcgagaagaacaatggagccatcctgttcgtcaaaaatgggttgtactacctgggcatcatgcccaagcagaagggccgttacaaggccctgtcattcgagcctaccgagaagacctcggagggcttcgacaagatgtactacgactatttcccggacgccgccaagatgatcccgaagtgctccacgcagctcaaagccgtcacggcccacttccagacgcataccacgccgatacttctgagcaacaacttcattgagccgctagagatcacgaaggagatatacgacctaaacaaccccgaaaaggagcccaagaagttccagacagcctacgctaagaagacaggtgatcagaagggatatagggaggcactctgcaagtggatcgacttcacgcgcgacttcctgtcgaaatatacaaagacgaccagcattgacctaagttctctccgcccatcctcccagtacaaggatctgggcgagtattatgcggagctgaacccattgctgtaccacatcagcttccagaggatcgccgagaaggagattatggacgcggtggagacggggaaactatacctgttccaaatatataacaaggacttcgctaaagggcaccacgggaagcccaacctgcacacactctactggacgggcttgttttcgccagaaaatttggccaagacttcgatcaagctcaacggccaggcggagttgttttaccgtcccaagtctcgcatgaagcgcatggcgcatcgcctcggagagaaaatgcttaacaagaagctcaaggatcagaagacgcccatacctgatacgttgtaccaggaattgtacgactacgtgaaccaccgcctatcgcacgacctctcagacgaggcccgcgccctcctcccaaacgtgattactaaggaggtttcccatgaaataatcaaggaccgacggttcaccagcgacaaattttttttccacgtgcctatcacgctcaattaccaggcggccaactccccatcgaagttcaaccagcgcgtgaacgcctaccttaaggagcacccggagaccccaatcatcgggatcgaccgtggcgagcggaacctgatctatattacggtgatcgatagcaccgggaagatcctggagcagcgctccctgaacacaatccagcagtttgactaccagaagaaactcgacaaccgggagaaggagcgcgtcgcagcccggcaagcatggagtgtggtcggcaccataaaggacctgaaacagggttacctaagtcaagttatccacgagatcgttgacctgatgatacactatcaagccgtagtcgtgctggagaacctcaacttcgggtttaagtccaagcgcaccggcatcgcggagaaggcggtgtaccagcagttcgagaagatgctgatcgacaagctgaactgcctggtgctcaaggactaccctgcggagaaggtcggcggggtcttgaacccgtaccagctaaccgaccagttcacgagcttcgccaaaatgggcacgcagtccggattcttgttttatgtcccggctccatatacaagtaagatcgacccgctgacagggtttgttgacccattcgtgtggaagaccatcaagaaccacgagagcaggaaacacttcttagagggcttcgacttcctgcattacgacgttaagacaggcgacttcatcctgcacttcaagatgaaccgcaacctgtcgttccagaggggcctgcccggcttcatgcccgcctgggatatcgtctttgagaagaatgagacgcagttcgacgcgaaggggacgccgttcatcgctggaaagcggatcgtgccggtcatcgagaaccaccgcttcacgggtcgctaccgagatttataccccgccaacgaactaattgcgctgctggaggagaaggggatcgtgttccgagatggcagcaacattctcccgaagctgctggagaacgacgactcgcacgctattgacacgatggtcgccctcatacggagcgtgcttcagatgcggaacagtaacgctgccacgggcgaggactacattaactcccccgtccgcgacctcaacggggtctgcttcgatagccgcttccagaacccggagtggcctatggatgcggacgcgaacggggcctaccacatcgccctcaagggccaactcctgctcaaccacttgaaggaaagcaaagacctcaaattgcagaatggcatcagtaaccaggactggctcgcgtacatccaggaactgagaaacgggtccaagaagcggcgtatcaagcaagattga

SEQ ID NO: 19 - DNA, 3987 bp, synthetic construct
  misc_feature 1..3987: note = Synthetic polynucleotide
  source 1..3987: mol_type = other DNA
atggcgggaagcaaaaagcgccggattaagcaagacacgcagttcgagggcttcacgaacctctaccaagtcagcaagaccctccggttcgagctgataccacagggaaagacgctcaagcacatccaggaacagggcttcatcgaggaggacaaggcgcgcaacgaccactacaaggagttgaaaccgatcatcgaccgcatctacaagacgtacgccgaccagtgcctccagctcgtgcagctcgactgggagaacctctccgccgccattgactcgtaccggaaggagaagactgaggagacccgcaacgccctgatcgaggagcaagcaacctaccggaacgccatccacgactacttcatcggccgcaccgacaacctcaccgacgcgatcaacaagcggcacgcggagatatacaaagggctgttcaaggcggagctgttcaacggcaaggtgctcaagcagctagggacggtgaccacgaccgagcacgagaacgcgctcctccgcagcttcgacaagttcaccacctacttcagcggcttctaccggaaccgcaagaatgtgttcagcgcggaggacatcagcacggccatcccgcaccgcatcgtccaggacaacttcccgaagttcaaggagaactgccacatcttcacccgcctgataaccgccgtcccctccctgcgggagcacttcgagaacgtcaaaaaggcaattgggatcttcgtctcgaccagcattgaggaggtgttcagcttccccttctacaaccagctcctcacccagacgcagatcgacctgtacaatcagttgctcggcgggataagccgcgaggcgggaaccgaaaaaatcaaggggctgaacgaagtgttgaacctcgccatccagaagaacgacgagaccgcgcacatcatcgcctccctgccccaccggttcatcccgctgttcaagcagatcctctctgaccggaacaccctgtccttcattcttgaggagttcaagtcggacgaggaggtcatccagagcttctgcaagtacaagacgctgctacggaacgagaacgtgctggagacggcggaggcactgttcaacgagctaaacagcatcgacctcacgcacatcttcatcagtcacaagaaactggagaccatctcctccgcgctgtgcgaccactgggacacgctcaggaacgcgctctacgagcgccgaatcagtgagctgacgggcaagatcacgaagtccgcgaaggagaaggtgcagcggtccctcaagcacgaggacatcaacctccaggagatcatctcagcggctgggaaagagctgtccgaggcgttcaagcagaaaacgagcgaaatcctgtcccacgcgcacgcggccctggatcagcctctgccgacgaccctcaagaaacaagaagaaaaggaaatcctcaagtcgcagctcgactcgctgctgggcctgtaccatctcctcgactggttcgccgtggacgagagcaacgaggtggaccccgagttctccgcgcggcttacggggatcaagctggagatggagcccagcctgtccttctacaacaaggcgcgcaactacgccaccaagaagccctacagcgtggagaagttcaagctcaacttccagatgcccactctcgcacgtgggtgggacgtcaaccgcgaaaaaaataatggggcgatcctgttcgtcaagaacggcctgtactacttgggcatcatgccgaaacagaagggccgctacaaggccctgagcttcgaaccgaccgagaaaacgagcgaggggttcgacaagatgtactacgactacttccccgacgccgcgaagatgattccaaagtgctccacgcagcttaaggccgtgacggcccacttccagacgcacacgaccccgatcctcctcagcaacaacttcatcgagcccctggagatcacgaaggagatatacgacctgaacaacccggagaaggagcccaagaaattccagaccgcctacgccaagaagacaggcgaccaaaagggttacagggaggccctctgcaagtggatcgacttcactagggacttcctgtccaagtacaccaagactacctctatcgacctgtccagcctccgcccgtcgtcccagtacaaagatttgggcgagtattacgcggagctgaacccactgctctaccacatcagcttccagcgcatcgcggagaaggagatcatggacgcagtggagacgggcaagctatacctatttcagatatacaacaaagacttcgctaagggacaccacggcaagcctaacctgcacaccctctactggacggggctcttcagcccggagaacctcgccaagacctcgatcaagctcaacggccaggccgagctgttctaccggcccaagtcccgcatgaagcggatggcccaccggctcggggagaaaatgctcaacaagaaattgaaggaccaaaaaacgccgatacccgacaccctataccaggagctgtacgactatgtgaaccaccgcctgagccacgacctcagcgacgaggcgcgggccctcctgccgaacgtcatcacaaaggaggtcagccacgagatcatcaaggaccggcgcttcacctccgacaagtttttctttcacgtgcccatcacgctcaactaccaggccgccaactcgccgtccaagttcaaccagcgcgtgaacgcctacctcaaggagcaccccgagaccccgatcatcgggattgaccgaggggagcggaacctcatctacatcaccgtcatcgacagcaccgggaagatccttgaacagcggtcgctcaacaccatccagcagttcgactaccagaagaaactcgacaaccgggagaaggagagagtggcggcccgccaggcttggtccgtcgtcgggacgattaaggacttgaaacaaggttacctgtcgcaagtgatccacgagatcgttgacctgatgatccactaccaagccgtcgtggtcctggagaacctcaacttcggcttcaagagcaaacgaaccggcatcgcggagaaggccgtgtaccagcagttcgaaaaaatgctgatcgacaagctgaactgcctcgtgctcaaggactaccccgctgagaaggtcggcggggtgctgaacccgtaccagctcactgaccagttcaccagcttcgcaaagatgggcacccagtccggcttcctgttctacgtgcctgcgccatacacctcgaagatcgacccgctcaccgggttcgtggaccccttcgtctggaagaccatcaagaaccacgagagccgcaagcacttcctggagggcttcgacttcctccactacgacgtcaagaccggggacttcatcctgcacttcaagatgaaccgcaacctcagtttccagcgcggcctgccggggttcatgcccgcttgggatatagtcttcgagaagaatgagacgcagttcgacgcgaagggcaccccgttcatcgccgggaagcgcatcgtgccggtcatcgagaaccaccggttcaccgggcgctaccgcgacctatacccggcgaacgagttgatcgccctcctggaggagaagggcatcgtgttccgcgacggctccaacatcctcccgaagctgctcgaaaacgacgactcccacgccatcgacacgatggtcgcgctgatccggtcggtgctccagatgcggaactccaacgccgcgacgggcgaggactacatcaacagtccggtccgcgatctgaacggcgtctgcttcgactcccggttccagaaccccgagtggccgatggacgcggacgcgaacggcgcataccacatcgccctaaaagggcaattgctgctcaaccacctcaaggaatccaaagacctaaagctccagaacggcatctccaaccaggactggctggcgtacatccaggaactgcggaacgggagcaaaaaacgtcggatcaagcaagattga

SEQ ID NO: 20 - DNA, 3987 bp, synthetic construct
  misc_feature 1..3987: note = Synthetic polynucleotide
  source 1..3987: mol_type = other DNA
atggcgggctccaagaaacgccggattaagcaagatacccagttcgaggggttcacgaacctctaccaagtgagcaagaccctccgattcgaactgattcctcaggggaagaccctcaagcacatccaggagcaagggttcatcgaggaggacaaggcgcggaacgaccactacaaggaactcaaacccatcatcgaccgcatctacaagacctacgccgatcagtgcctccagctcgtgcagttggactgggagaacctcagcgcggccattgactcctaccggaaggagaaaacggaggagacgcgcaacgcgctcatcgaggaacaggcaacctatcgcaacgccatccacgactacttcatcgggaggactgacaacctcactgacgcgattaacaagcgccacgcggagatatacaagggactcttcaaagcggagctgtttaacggcaaggttctcaagcaactcggcactgtgaccacgaccgagcatgagaacgccctgctccgctccttcgacaagttcaccacctacttctccgggttctaccgcaaccgcaagaatgtcttcagcgcggaggacatcagcacggccattccacatcgaatcgtccaagataacttcccgaagttcaaggagaactgccacatcttcacccgactcattactgctgtaccgtcgttacgcgaacacttcgagaacgtcaagaaggcaattggaatcttcgtctctacgtcaatagaggaggtgttcagcttccctttctacaaccagctccttacgcagacccagatagacctgtacaatcagctcctcggtgggatcagccgggaggcggggactgagaagattaaagggctcaacgaggtcttgaacctggccatccaaaaaaacgatgagacggcgcacatcatcgcctcgctgccccaccggttcatcccgctgttcaagcagatcctcagtgacaggaacaccttgagctttatcctagaggagttcaagagcgacgaggaggtgatccagagcttctgcaagtacaaaaccctgctgaggaacgagaacgtcctggagacggcggaggcgctgttcaacgagctgaactctatcgacttaactcacatattcatctcgcacaagaagctggagactattagctctgcactctgcgaccactgggacaccctccgcaacgcgctctacgagcgccgcatctcggagctgaccgggaagatcaccaaatccgcgaaggaaaaggtccagcgttccctcaaacacgaggatattaacttacaggagattatctcagcggctgggaaggagttgtcagaggcgttcaagcagaaaacttccgagatcctgagccacgcgcacgcagcgctcgaccagcctctgcccaccaccctcaaaaagcaggaagaaaaagagatcctcaagagccagttggactccctgctggggctctatcaccttctcgactggttcgccgtcgatgagtcgaacgaggtggaccccgagttctccgcccggctgaccggcatcaagctagagatggagccgtccctcagcttctacaataaggcccgcaactacgcgaccaaaaaaccctacagcgtggagaagttcaagctgaacttccagatgccgaccttagcacgcggttgggacgtaaacagggagaagaacaatggagccatcctgttcgtcaagaacgggctttactacctcgggataatgcccaagcagaagggccgctacaaggccctttccttcgagccgacggagaaaacctccgaggggttcgacaagatgtactacgactacttccccgacgccgccaagatgatcccgaagtgctcaacgcagctaaaagccgtgaccgcccacttccagacccacacgacgccgatcctgctgagcaacaacttcatcgagccccttgagatcactaaggagatatacgacctgaacaaccccgagaaggagcccaagaagtttcaaaccgcctacgccaaaaaaactggcgaccaaaagggctacagggaggcgctgtgtaagtggatcgacttcacacgcgacttcctttcgaagtatacgaagacaacctctattgacctgagcagcctgcgtcctagctcccagtacaaagatttgggcgagtactacgcggagcttaatccactactctaccacatctcattccagcgcatcgctgagaaggaaatcatggacgcggtggagacaggcaaactgtacctcttccagatatacaacaaagacttcgctaaggggcaccacgggaagcccaaccttcatacgctctactggacgggcctattcagccccgaaaatctggccaagacctccatcaagctgaacggccaagcggagctgttctacagacccaagagccggatgaagcggatggcccacaggctcggcgagaaaatgcttaacaaaaagttgaaggaccagaaaacccctatccccgacaccctctaccaggaactgtacgactacgtgaaccacaggctctcgcacgacctttccgacgaggcccgtgccctactcccgaacgtcattaccaaagaggtttcgcacgagatcatcaaggaccggcggttcacgagcgacaagtttttctttcacgtccccatcacccttaactaccaggcggccaactccccatccaagttcaaccagcgtgtgaatgcctacctcaaggagcacccagagaccccgatcattgggatcgaccggggcgagcggaacctgatctacatcaccgtcatcgactcgacgggcaagattcttgagcagagatcgttgaataccatacagcagttcgactaccagaagaaactcgacaaccgcgagaaggagcgcgtggcggcccgccaggcgtggtccgtcgttgggacgattaaggacttgaaacaaggttatctgtcccaagtcatccacgagatcgttgatctgatgatccactatcaggcagtggtggtgctggagaatctcaacttcggcttcaagagtaagcggacgggaatcgccgagaaggccgtgtaccagcagttcgagaagatgctgatcgacaagctcaactgccttgtgctgaaagactacccggccgagaaggtcggcggcgtcctcaacccgtaccaacttaccgaccagttcacctccttcgccaagatgggcactcagtccgggttcttgttctacgtccccgcaccttacacctctaagatcgaccctctgactggcttcgtagatccattcgtgtggaagaccattaagaaccacgagagccgcaagcacttcctggagggcttcgacttcctgcactacgacgtgaagaccggggacttcatccttcacttcaagatgaaccggaacctcagcttccagcggggcctgccggggttcatgcccgcctgggacatcgtgttcgagaagaacgagacccagttcgacgcgaagggcacgcccttcatcgccgggaagcgtatcgtgccggtgatcgagaaccatcgtttcacgggtcgctaccgtgacctctacccggcgaacgagcttatcgcactcctggaggagaagggcatcgtcttccgggacggctccaacatcctcccgaaactgctggaaaacgacgactctcacgccatcgacacgatggtggccctcatccggtccgtgctccaaatgcggaacagcaacgccgccaccggtgaggactacatcaacagcccggtccgggatctgaacggggtgtgcttcgattcgcggttccagaatcctgagtggccgatggacgcggatgcaaacggggcgtaccacatcgcgctcaagggccagttacttctgaaccaccttaaggagtctaaagatttgaaactccagaacgggatctcgaaccaggactggctggcctacatccaagagttgcggaacggcagcaagaagcggcggattaagcaagattag

SEQ ID NO: 21 - DNA, 1592 bp, Medicago truncatula
  source 1..1592: mol_type = other DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacttactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagaggtgccgttggcgtaatataacagccaatcgatttttgctataaaagcaaatcaggtaaactaaacttcttcattcttttcttccccatcgctacaaaaccggttcctttggaaaagagattcattcaaacctagcacccaattccgtttcaaggtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagg

SEQ ID NO: 22 - DNA, 2000 bp, Zea mays
  source 1..2000: mol_type = other DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttc

SEQ ID NO: 23 - protein, 228 aa, Rattus norvegicus
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - DNA, 621 bp, Petromyzon marinus
  source 1..621: mol_type = other DNA
acagatgcagagtatgtgagaattcacgaaaagctggacatctataccttcaagaagcagttctttaacaataagaagtctgtgagccataggtgctacgtgctgttcgagctgaagagaaggggtgaaagaagggcatgtttttgggggtatgctgtgaacaagccccagtctggaactgagagaggcattcacgccgaaattttcagcatcagaaaggtggaggaatacctgagggataaccctggacagtttacaattaattggtattctagctggtctccatgcgctgactgtgccgagaagatcctggaatggtacaaccaggagctgagaggaaatggccataccctgaagatttgggcctgcaagctgtactatgaaaagaacgcaagaaatcagatcggactgtggaacctgagggataatggtgtggggctgaacgtgatggtgtccgagcactatcagtgctgtagaaagattttcattcagtcctcacataatcagctgaacgagaatagatggctggaaaagactctgaagagggctgagaagagaaggtccgaactgtcaattatgatccaggtgaagatcctgcacaccactaagtcacctgccgtg

SEQ ID NO: 26 - protein, 160 aa, synthetic construct
  REGION 1..160: note = Synthetic polypeptide
FERNYDPRELRKETYLLYEIKWGKSGKLWRHWCQNNRTQHAEVYFLENIFNARRFNPSTHCSITWYLSWSPCAECSQKIVDFLKEHPNVLEIYVARLYYHEDERNRQGLRDLVNSGVTIRIMDLPDYNYCWKTFVSDQGGDEDYWPGHFAPWIKQYSLKL

SEQ ID NO: 27 - protein, 207 aa, synthetic construct
  REGION 1..207: note = Synthetic polypeptide
TDAEYVRIHEKLDIYTFKKQFSNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWVCKLYYEKNARNQIGLWNLRDNGVGLNVMVSEHYQCCRKIFIQSSHNQLNENRWLEKTLKRAEKRRSELSIMFQVKILHTTKSPAV

SEQ ID NO: 28 - protein, 228 aa, synthetic construct
  REGION 1..228: note = Synthetic polypeptide
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 29 - protein, 162 aa, synthetic construct
  REGION 1..162: note = Synthetic polypeptide
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYPENERNRQGLRDLVNSGVTIRIMDLPDYNYCWKTFVSDQGGDEDYWPGHFAPWIKQYSLKL

SEQ ID NO: 30 - protein, 166 aa, Escherichia coli
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 31 - protein, 166 aa, synthetic construct
  REGION 1..166: note = Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 32 - protein, 166 aa, synthetic construct
  REGION 1..166: note = Synthetic polypeptide
SEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 33 - protein, 166 aa, synthetic construct
  REGION 1..166: note = Synthetic polypeptide
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 34 - protein, 166 aa, synthetic construct
  REGION 1..166: note = Synthetic polypeptide
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 35 - protein, 1763 aa, synthetic construct
  REGION 1..1763: note = Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 1565 aa, synthetic construct
  REGION 1..1565: note = Synthetic polypeptide
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 37 - protein, 1565 aa, synthetic construct
  REGION 1..1565: note = Synthetic polypeptide
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 38 - protein, 364 aa, synthetic construct
  REGION 1..364: note = Synthetic polypeptide
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 39 - protein, 167 aa, synthetic construct
  REGION 1..167: note = Synthetic polypeptide
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 40 - protein, 167 aa, synthetic construct
  REGION 1..167: note = Synthetic polypeptide
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 41 - protein, 83 aa, unidentified
  source 1..83: note = Bacillus phage AR9
TNLSDIIEKETGKQLVIQESILMLPEEVEEVIGNKPESDILVHTAYDESTDENVMLLTSDAPEYKPWALVIQDSNGENKIKML

SEQ ID NO: 42-44 - skipped (3 entries)

SEQ ID NO: 45 - protein, 24 aa, synthetic construct
  REGION 1..24: note = Synthetic peptide
EELLSKNYHLENEVARLKKGSGSG

SEQ ID NO: 46 - protein, 241 aa, synthetic construct
  REGION 1..241: note = Synthetic polypeptide
EEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSGEELLSKNYHLENEVARLKKGSGSG

SEQ ID NO: 47 - protein, 277 aa, synthetic construct
  REGION 1..277: note = Synthetic polypeptide
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGS

SEQ ID NO: 48 - DNA, 66 bp, Saccharomyces bayanus
  source 1..66: mol_type = other DNA
ttcttgtcgtacttatagatcgctacgttatttcaattttgaaaatctgagtcctgggagtgcgga

SEQ ID NO: 49 - protein, 605 aa, Homo sapiens
MSGWESYYKTEGDEEAEEEQEENLEASGDYKYSGRDSLIFLVDASKAMFESQSEDELTPFDMSIQCIQSVYISKIISSDRDLLAWFYGTEKDKNSVNFKIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPLKEACRAYGLKSGLKKQELLEALTKHFQD

SEQ ID NO: 50 - protein, 482 aa, synthetic construct
  REGION 1..482: note = Synthetic polypeptide
MVRSGNKAAWLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISERHSIHWPCRLTIGSNLSIRIAAYKSILQERVKKTTWDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDIWAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAK

SEQ ID NO: 51 - DNA, 10 bp, Methanobacterium thermoautotrophicum
  source 1..10: mol_type = other DNA
aatttttgga

SEQ ID NO: 52 - protein, 83 aa, Methanobacterium thermoautotrophicum
GSVIDVSSQRVNVQRPLDALGNSLNSPVIIKLKGDREFRGVLKSFDLHMNLVLNDAEELEDGEVTRRLGTVLIRGDNIVYISP

SEQ ID NO: 53 - DNA, 25 bp, Emesvirus zinderi
  source 1..25: mol_type = other DNA
gcgcacatgaggatcacccatgtgc

SEQ ID NO: 54 - protein, 116 aa, Emesvirus zinderi
MASNFTQFVLVDNGGTGDVTVAPSNFANGIAEISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 55 - DNA, 26 bp, Pepevirus rubrum
  source 1..26: mol_type = other DNA
ataaggagtttatatggaaaccctta

SEQ ID NO: 56 - protein, 127 aa, Pepevirus rubrum
MSKTIVLSVGEATRTLTEIQSTADRQIFEEKVGPLVGRLRLTASLRQNGAKTAYRVNLKLDQADWDCSTSVCGELPKVRYTQVWSHDVTIVANSTEASRKSLYDLTKSLVATSQVEDLVVNLVPLGR

SEQ ID NO: 57 - DNA, 19 bp, unidentified
  source 1..19: mol_type = other DNA; note = Shigella phage
ctgaatgcctgcgagcatc

SEQ ID NO: 58 - protein, 62 aa, unidentified
  REGION 1..62: note = Shigella phage
MKSIRCKNCNKLLFKADSFDHIEIRCPRCKRHIIMLNACEHPTEKHCGKREKITHSDETVRY

SEQ ID NO: 59 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 60 - protein, 1367 aa, synthetic construct
  REGION 1..1367: note = Synthetic polypeptide
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 61 - DNA, 4101 bp, synthetic construct
  misc_feature 1..4101: note = Synthetic polynucleotide
  source 1..4101: mol_type = other DNA
gacaagaagtacagcatcgggctggcgatcgggaccaactccgtcggctgggctgtgattaccgacgagtacaaggtgccatccaagaagttcaaggtcctcggcaacactgaccggcacagcattaagaagaacctgattggggcgctgctgttcgattcgggggagactgcggaggcgaccaggctgaagcggactgcgcgccggaggtacaccaggaggaagaatcggatctgctacctccaggagattttctcgaatgagatggccaaggtggacgattccttcttccatcgcctggaggagtcgttcctcgttgaggaggacaagaagcatgagaggcatcccattttcgggaatatcgttgacgaggtggcttaccatgagaagtacccgaccatctaccatctgcggaagaagctcgtcgattcgaccgataaggccgacctgcggctgatctacctggccctcgcgcacatgattaagttccggggccatttcctcatcgagggcgacctcaacccggacaactcggacgtggataagctcttcattcagctcgtgcagacatacaaccagctcttcgaggagaatcccattaacgcctcgggggtcgacgctaaggctattctctcggctcggctgtcgaagtcgcgccggctggagaatctcattgcccagctcccaggcgagaagaagaacggcctcttcggcaacctgattgccctgtcgctggggctcacaccgaatttcaagtcgaacttcgacctcgccgaggacgctaagctccagctcagcaaggatacttacgatgatgacctcgataacctgctcgcccagattggggatcagtacgcggatctgttcctcgcggccaagaatctcagcgatgctattctcctgtcggacattctccgcgtcaacacagagattactaaggccccactgtcggcgagcatgattaagaggtacgatgagcatcatcaggacctgacactgctcaaggcgctggtccggcagcagctccccgagaagtacaaggagattttcttcgatcagtcaaagaatgggtacgcgggctacattgatggcggcgcgtcccaggaggagttctacaagttcattaagcccatcctggagaagatggacgggaccgaggagctgctggtgaagctcaatcgggaggacctgctccggaagcagcgcacattcgacaatggctcgattcctcaccagattcacctgggcgagctgcacgccattctccgcaggcaggaggacttctacccgttcctcaaggacaaccgcgagaagatcgagaagatcctgaccttccggattccatactacgtggggccgctcgcgcgggggaactcccggttcgcgtggatgactcgcaagtccgaagaaacgattacaccgtggaatttcgaggaggtcgtcgacaagggcgctagtgcgcagtcattcattgagaggatgaccaatttcgataagaacctgcctaacgagaaggtgctgccgaagcattcgctgctctacgagtacttcaccgtttacaatgagctgaccaaggtgaagtatgtgactgagggcatgaggaagccagcgttcctgagcggcgagcagaagaaggctatcgtggacctgctcttcaagactaaccggaaggtgactgtgaagcagctcaaggaggactacttcaagaagattgagtgcttcgattccgttgagattagcggggtggaggatcggttcaatgcttcgctcgggacataccacgatctcctgaagatcattaaggataaggacttcctcgacaacgaggagaacgaggacattctcgaagatattgtcctgaccctcaccctcttcgaggatcgggagatgatcgaggagaggctcaagacatacgctcatctgttcgatgataaggtcatgaagcagctgaagcgcaggcggtacacagggtgggggcggctgagccggaagctgatcaacgggattcgggataagcagtccgggaagacaattctcgacttcctcaagtccgacgggttcgctaaccggaacttcatgcagctcattcatgatgactcgctgacattcaaggaggatattcagaaggcgcaggtttcggggcagggcgactcgctccacgagcatattgcgaatctggcgggctcccccgcgattaagaagggcattctgcaaaccgtcaaggtggttgatgagctggtcaaggtcatggggcggcataagccagagaatattgtcatcgagatggcgcgggagaatcagaccacacagaaggggcagaagaactcacgggagcggatgaagcgcatcgaggagggcatcaaggagctggggtcgcagatcctgaaggagcatcccgtggagaacactcagctgcaaaatgagaagctgtacctctactacctccagaacgggagggacatgtatgtggatcaggagctggatattaataggctgagcgattacgatgtcgaccacattgtcccacagtcgttcctgaaggacgacagcattgacaacaaggtgctgacccgctcggataagaacaggggcaagagcgataatgttccaagcgaggaggttgtgaagaagatgaagaactactggcggcagctcctgaacgcgaagctcatcacacagcggaagttcgacaacctcaccaaggctgagcgcgggggcctgagcgagctggacaaggcggggttcattaagaggcagctggtcgagacacggcagattacaaagcatgttgcgcagattctcgattcccggatgaacaccaagtacgatgagaacgataagctgattcgggaggtcaaggtaattaccctgaagtccaagctggtgtccgacttcaggaaggacttccagttctacaaggttcgggagatcaacaactaccaccacgcgcatgatgcctacctcaacgcggtcgtggggaccgctctcatcaagaagtacccaaagctggagtcagagttcgtctacggggattacaaggtttacgacgtgcggaagatgatcgctaagagcgagcaggagattggcaaggctaccgctaagtacttcttctactccaacatcatgaacttcttcaagacagagattaccctcgcgaatggcgagatccggaagaggcccctcatcgagacaaatggggagacaggggagattgtctgggataaggggcgggatttcgcgaccgtccggaaggtcctgtcgatgccccaggttaatattgtcaagaagactgaggtccagactggcggcttctcaaaggagtcgattctcccaaagaggaactccgataagctcattgctcggaagaaggattgggaccccaagaagtacgggggattcgactcccccactgttgcttactctgttctggttgttgctaaggtggagaaggggaagtcgaagaagctgaagagcgtgaaggagctgctcgggattacaattatggagaggtcatccttcgagaagaatcccatcgacttcctggaggccaagggctacaaggaggtgaagaaggacctgattattaagctgcccaagtactcgctcttcgagctggagaatgggcggaagcggatgctggcgtccgcgggggagctgcaaaaggggaacgagctggcgctcccctccaagtatgtgaacttcctctacctggcgtcgcactacgagaagctgaaggggtccccagaggataatgagcagaagcagctcttcgtcgagcagcataagcactacctggacgagattatcgagcagattagcgagttctcgaagcgggtcatcctcgcggatgcgaacctggataaggtgctcagcgcctacaataagcaccgggacaagccgattcgggagcaggcggagaatattattcacctcttcacactcaccaacctcggggcaccagctgcgttcaagtacttcgacactactatcgaccggaagcggtacacctcgacgaaggaggtgctcgacgccaccctcattcaccagtcgatcacaggcctgtacgagacacggattgacctgtcccagctcgggggcgac

SEQ ID NO: 62 - DNA, 4101 bp, synthetic construct
  misc_feature 1..4101: note = Synthetic polynucleotide
  source 1..4101: mol_type = other DNA
gacaagaagtactccattggcctggcgattgggacaaactcggtggggtgggccgtgattacggatgagtacaaggttccaagcaagaagttcaaggtcctcgggaacacagatcggcattcgattaagaagaatctcattggggcgctcctcttcgactcgggggagacagcggaggctaccaggctcaagcggacagccaggcggcggtacacaaggcggaagaatcgcatctgctacctccaggagattttctcgaatgagatggcgaaggtggacgacagcttcttccatcggctggaggagtccttcctggtggaggaggataagaagcacgagaggcatccaattttcgggaacatcgtggacgaggttgcgtaccatgagaagtaccctacaatctaccatctgcggaagaagctggttgactccacagacaaggcggacctgaggctgatctacctcgctctggcccacatgattaagttccgcgggcatttcctgatcgagggggacctgaatcccgacaattcggatgtggacaagctcttcatccagctggtgcagacctacaaccagctgttcgaggagaatcccatcaatgcgtcgggcgttgacgctaaggccattctgtccgctaggctgtcgaagagcaggaggctggagaacctgatcgcccagctgccaggcgagaagaagaatgggctcttcgggaatctgattgcgctctccctggggctgacaccgaacttcaagagcaatttcgatctggctgaggacgcgaagctccagctctcgaaggacacttacgacgatgacctcgataacctcctcgcgcagatcggggaccagtacgctgatctcttcctcgccgctaagaacctctcggatgctatcctgctctccgacattctccgggttaataccgagattacaaaggccccactgtcggcgtccatgatcaagcggtacgatgagcatcatcaggatctcaccctgctcaaggccctcgtgcggcagcagctgcccgagaagtacaaggagattttcttcgaccagagcaagaatgggtacgctggctacattgacggcggggcctcacaggaggagttctacaagttcatcaagccaatcctggagaagatggatgggacagaggagctgctggtgaagctcaaccgggaggatctgctcaggaagcagcggacgttcgacaacgggtcgattccccatcagatccacctgggggagctgcacgcgatcctgcgccggcaggaggatttctaccctttcctgaaggataatcgggagaagatcgagaagattctcaccttccggattccctactacgtcgggccactcgcgcggggcaatagcaggttcgcctggatgacacggaagagcgaggagacaatcaccccctggaacttcgaggaggttgtcgacaagggggcgtccgcccagtcattcattgagcggatgaccaatttcgacaagaatctgccaaatgagaaggttctcccaaagcatagcctcctctacgagtacttcactgtttacaacgagctgaccaaggtgaagtatgtgaccgagggcatgcggaagcccgcgttcctgtccggcgagcagaagaaggccattgtggacctcctgttcaagaccaatcgcaaggtcacagtcaagcagctcaaggaggattacttcaagaagatcgagtgcttcgactcggttgagattagcggggtggaggatcggttcaacgcgagcctcggcacttaccacgacctcctgaagatcatcaaggataaggacttcctcgacaacgaggagaacgaggatattctggaggacatcgtgctcaccctgacgctgttcgaggatcgggagatgatcgaggagcgcctgaagacctacgctcatctcttcgatgataaggtcatgaagcagctgaagaggaggcggtacaccgggtggggccgcctgagcaggaagctcattaacgggatcagggacaagcagagcggcaagaccatcctggacttcctcaagagcgatggcttcgccaaccggaatttcatgcagctcatccacgacgactccctcaccttcaaggaggacattcagaaggctcaggtcagcggccagggcgactcgctgcatgagcacatcgctaacctggcgggcagcccagccatcaagaagggcatcctccagacagtgaaggtcgtggatgagctggtgaaggtcatgggccggcataagcccgagaatattgtgattgagatggcgcgggagaatcagaccactcagaagggccagaagaactcgcgggagcgcatgaagaggatcgaggaggggattaaggagctgggcagccagattctcaaggagcaccccgtggagaatacccagctccagaacgagaagctgtacctctactacctccagaatgggcgggacatgtatgttgatcaggagctggacatcaatcgcctctcggattacgacgtggaccacatcgtgccccagagcttcctgaaggatgatagcatcgacaataaggtcctgacccgctccgacaagaatcgcggcaagagcgacaacgtgccgagcgaggaggtcgtgaagaagatgaagaactactggcggcagctgctgaacgcgaagctcattacacagcggaagttcgataacctgacgaaggcggagaggggcggcctctccgagctggacaaggcgggcttcattaagaggcagctcgtggagactcgccagatcaccaagcacgtggctcagatcctcgatagccggatgaatacgaagtacgatgagaatgacaagctcatccgggaggtgaaggtaatcaccctgaagtcaaagctcgttagcgatttccggaaggacttccagttctacaaggtgcgggagattaacaactaccatcatgcgcacgatgcgtacctcaatgcggtggtgggcacagccctgattaagaagtaccccaagctggagagcgagttcgtctacggggactacaaggtgtacgatgttcggaagatgatcgccaagagcgagcaggagattgggaaggccaccgctaagtacttcttctactcgaatattatgaatttcttcaagaccgagatcacactcgctaatggggagattcggaagcggcccctcatcgagactaacggggagactggcgagattgtgtgggacaaggggcgcgacttcgctaccgtgcgcaaggtcctctcgatgccccaggttaatattgttaagaagacagaggtgcagacgggcgggttctccaaggagtctatcctgccgaagcggaactcggacaagctgatcgcccgcaagaaggattgggaccccaagaagtacgggggattcgatagcccaaccgtggcttacagcgtcctggtggtcgccaaggttgagaaggggaagtcgaagaagctcaagagcgttaaggagctgctgggcatcaccatcatggagcggtccagcttcgagaagaatcctatcgacttcctggaggctaaggggtacaaggaggtcaagaaggacctgatcattaagctgcccaagtactctctgttcgagctggagaacgggaggaagcggatgctggcgtctgctggcgagctacagaagggcaatgagctggcgctcccctcgaagtatgtcaacttcctctacctggcttcccattacgagaagctgaagggctcgcccgaggataatgagcagaagcagctcttcgtggagcagcacaagcactacctcgacgagatcattgagcagatttcggagttctcgaagcgggtcattctcgcggacgcgaacctcgacaaggtcctctcggcgtacaacaagcaccgggacaagcccatccgggagcaggccgagaacattatccacctcttcacactgaccaacctcggcgctcccgccgcgttcaagtacttcgacaccaccattgaccgcaagagatacacatccaccaaggaggtgctggacgcgaccctcatccaccagagcatcacaggcctctacgagacacggatcgacctctcgcagctcgggggcgat

SEQ ID NO: 63 - DNA, 4092 bp, synthetic construct
  misc_feature 1..4092: note = Synthetic polynucleotide
  source 1..4092: mol_type = other DNA
gacaagaagtactcgatcggcctggcgattggcacaaacagcgtggggtgggctgtgatcactgatgagtacaaggtgccatcgaagaagttcaaggtgctggggaatacagaccggcattcgatcaagaagaatctcattggcgctctcctcttcgattccggcgagactgctgaggcgacccgcctgaagcgcaccgcccggcggcgctacactcggcggaagaataggatttgctacctccaggagattttctcgaatgagatggccaaggtggatgacagcttcttccaccgcctggaggagtcgttcctggtcgaggaggacaagaagcatgagcggcaccctatcttcgggaatatcgttgatgaggtcgcctaccacgagaagtaccccactatctaccatctccgcaagaagctcgtggacagcacagataaggccgacctccgcctgatctacctcgccctcgcgcacatgattaagttccgggggcacttcctcattgagggggatctgaatcccgataactccgacgtggacaagctgttcatccagctggtgcagacatacaaccagctgttcgaggagaatcccatcaacgcgagcggcgtggacgctaaggccattctgtcggctaggctctcgaagtcgaggcggctggagaacctgattgcgcagctccccggcgagaagaagaacgggctgttcgggaatctcatcgccctctccctcggcctcacaccaaacttcaagagcaatttcgacctggctgaggacgctaagctgcaactctcaaaggatacatacgatgacgacctggacaatctcctggctcagatcggcgaccagtacgctgacctgttcctcgcggccaagaatctgtcggacgcgattctcctcagcgacatcctgcgcgtcaataccgagattacgaaggctccactgtctgcgtcaatgattaagcggtacgatgagcatcaccaggatctgaccctcctgaaggcgctcgtgcggcagcagctgcccgagaagtacaaggagattttcttcgatcagagcaagaatggctacgccggctacatcgacgggggcgcgagccaggaggagttctacaagttcatcaagcccatcctggagaagatggacggcaccgaggagctactcgtgaagctcaatcgggaggatctcctccggaagcagcggacattcgataacgggtctatcccacaccagatccacctcggcgagctgcatgcgattctgcggcggcaggaggatttctaccctttcctgaaggacaaccgggagaagatcgagaagatcctcacattccggattccatactacgtcggccccctggcgaggggcaatagccggttcgcgtggatgacaaggaagtccgaggagactattaccccgtggaatttcgaggaggtggttgacaagggcgcttccgcgcagagcttcattgagcggatgacaaacttcgacaagaatctccccaacgagaaggtcctgccgaagcatagcctcctgtacgagtacttcaccgtctacaatgagctaactaaggtcaagtatgtgacagagggcatgaggaagccagccttcctctcaggcgagcagaagaaggccattgtggacctcctgttcaagacaaaccgcaaggtgacagtgaagcagctgaaggaggattacttcaagaagattgagtgcttcgactcagtggagatttcaggcgtggaggatcggttcaacgcgagcctggggacttaccacgacctgctgaagattattaaggacaaggacttcctggataacgaggagaatgaggacatcctggaggatattgtgctcaccctcaccctgttcgaggacagggagatgattgaggagaggctcaagacctacgcgcacctgttcgatgacaaggtcatgaagcagctgaagaggcggcgctacactgggtggggccgcctgtcgcggaagctgatcaacggcattcgggataagcagtccgggaagaccattctggatttcctgaagtcggacggcttcgccaacaggaatttcatgcagctgatccacgacgactccctcaccttcaaggaggacattcagaaggcccaggttagcggccagggggactcactccacgagcatattgccaatctggccggctctccagctatcaagaagggcatcctgcaaacagttaaggttgttgacgagctggttaaggtcatggggcggcataagcccgagaacattgtcatcgagatggctcgggagaaccagacaactcagaagggccagaagaactccagggagcgcatgaagcggattgaggagggcattaaggagctggggtcccagatcctcaaggagcaccctgtcgagaacactcagctgcaaaacgagaagctctacctgtactacctccagaacgggcgggatatgtatgtggatcaggagctggacatcaacaggctctccgactacgacgtggatcacattgtcccacagtctttcctcaaggatgattccatcgacaacaaggtgctgacgcgcagcgacaagaatagggggaagtcggacaacgttccgagcgaggaggtcgtgaagaagatgaagaattactggaggcagctcctgaatgcgaagctgatcactcagaggaagttcgacaatctgacaaaggcggagaggggcgggctctcggagctggataaggcgggcttcatcaagcggcagctcgttgaaacccggcagatcaccaagcatgtcgcccagatcctcgatagccgcatgaacaccaagtacgatgagaacgacaagctcattcgggaggttaaggtcattacgctgaagtccaagctcgtcagcgacttcaggaaggatttccagttctacaaggttcgggagattaacaactaccaccacgcgcatgatgcgtacctgaacgctgttgtcggcactgctctcatcaagaagtacccaaagctggagtccgagttcgtctacggggactacaaggtctacgatgtccggaagatgatcgccaagtcggagcaggagatcgggaaggctactgcgaagtacttcttctacagcaacattatgaatttcttcaagacggagattacgctggcgaacggggagattaggaagaggcccctcattgagactaatggggagacaggcgagattgtttgggacaagggccgcgacttcgcgactgtgcggaaggtcctgtccatgccacaggtgaatattgttaagaagacagaggtgcagactgggggcttctcgaaggagagcattctcccaaagcggaacagcgataagctcatcgcgcgcaagaaggattgggaccctaagaagtacggcggcttcgattctcccactgtggcctactccgttctcgtggttgccaaggttgagaaggggaagtcgaagaagctgaagtcggtcaaggagctgctcgggattacaatcatggagcggagcagcttcgagaagaaccctattgatttcctggaggccaagggctacaaggaggttaagaaggatctcattatcaagctccctaagtactctctgttcgagctggagaatggccggaagaggatgctggcctcggctggcgagctacagaaggggaatgagctggccctcccgtcgaagtatgtgaatttcctgtacctcgcgtcgcactacgagaagctcaagggcagcccggaggataatgagcagaagcagctcttcgtggagcagcataagcactacctggacgagatcattgagcagatcagcgagttctcgaagcgggttattctggctgatgctaacctggacaaggttctgagcgcctacaataagcatcgcgacaagccgattcgcgagcaggcggagaatattatccacctgttcaccctcactaacctcggggctcccgcggccttcaagtacttcgataccacaatagataggaagcggtacacctcgacgaaggaggtcctcgacgccacactcatccatcagtcgattacaggcctgtacgagacacggattgacctctcgcagctg

SEQ ID NO: 64 - DNA, 4101 bp, synthetic construct
  misc_feature 1..4101: note = Synthetic polynucleotide
  source 1..4101: mol_type = other DNA
gacaagaagtattccataggcctggctatcggcaccaacagcgtgggctgggccgtcatcaccgacgagtacaaagtgccgagtaaaaagttcaaagtgctcggcaacaccgaccgccactccataaagaaaaacctgatcggggcgctcctgttcgacagcggcgagacggcggaggccacccgcttgaaacgcacggcccgacggcgctacacgcggcgcaagaaccggatctgttacctacaggagattttctctaacgagatggcgaaggtggacgactcgttctttcaccgcctcgaagagtccttcctcgtggaggaggacaagaaacacgagcgccacccgatcttcggcaacatcgtggacgaggtggcctaccacgagaagtacccgaccatctaccacctccggaagaaactcgtggacagcacggacaaggccgacctgaggctcatctacctcgccctggcgcacatgattaagttccggggccacttcctgatcgagggcgacctgaacccggacaacagcgacgtggacaagctgttcatccagctagtccagacctacaaccagcttttcgaggaaaaccccatcaacgccagcggggtggacgcgaaggcgatcctgtccgcccggctgagcaagtcccggcggctggagaacctcatcgcgcagttgcccggcgagaagaagaacgggctgttcgggaacctgatcgccctctccctggggctcaccccgaacttcaagtccaacttcgacctcgccgaggacgccaaactacagctgagcaaggacacctacgacgacgacctcgacaacctgctggcccagatcggggaccagtacgcagacctgttcctcgccgccaagaacctctccgacgccatcctgctgtcggacatcctgcgggtgaacacggagatcacgaaggccccgctctcggcctcgatgattaaacgctacgacgagcaccaccaggacttgaccctcctcaaggcgctggtccgccagcagcttcccgagaagtacaaggaaatctttttcgatcagagcaagaacgggtacgccgggtacatcgacggcggggcgtcccaggaggagttctacaagttcatcaagcccatcctggagaaaatggacgggaccgaggagctgctcgtgaagctcaaccgcgaagatttgctccgcaagcagcgcacgttcgacaacgggtcgatcccgcaccagatccacctgggcgagctgcacgcgatcctcaggcgtcaggaagacttctaccccttcctcaaggacaaccgcgagaagatagagaagattctgaccttcagaattccttattacgtgggcccgctggctcggggcaactcgcgcttcgcctggatgacgcgcaagtccgaggagaccatcaccccgtggaacttcgaggaggtggtggataagggtgcctcggcccagtccttcatcgagcggatgaccaacttcgacaagaacctgccgaacgagaaggtgctccccaagcacagcctgctctacgaatatttcacggtgtacaacgagctgacgaaggtcaagtacgtgaccgagggaatgaggaaacctgcattcctctccggggagcagaagaaagccatagtcgacctcctgttcaagaccaaccggaaggtcaccgtcaagcagctcaaggaggactacttcaagaagatcgagtgcttcgattcagtggagatcagcggcgtcgaggaccggttcaacgccagcctgggcacctaccacgacctgctcaagatcatcaaggacaaggacttcctcgacaacgaggagaacgaggacatcctggaggacatcgtgctgaccctgacgctcttcgaggaccgcgagatgatcgaggagcgcctcaagacctacgcccacctgttcgacgacaaggtgatgaagcagctcaagcggcggagatatactgggtggggccgcctctcccggaagctcattaacggtatcagggataagcagtccgggaagacgatcctcgacttcctcaagtcggacgggttcgccaaccgcaacttcatgcagctcatccacgacgactccctgacgttcaaggaggacatccagaaggcccaagtgtctggtcaaggtgactcgctccacgagcacatcgccaacctcgcgggcagcccggccatcaagaagggaatactccagaccgtcaaggtggtggacgagctggtgaaggtcatgggccgccacaagccggagaacatcgtcatcgagatggcgcgggagaaccagaccacgcagaaggggcagaaaaatagccgtgagcgcatgaagcgcatcgaggaggggattaaggagttgggcagccagatcctcaaggagcaccctgtggagaacacgcagttgcaaaacgagaagctctacctgtactacctccagaacgggagggatatgtacgtggaccaagaactggacatcaaccgcctgtccgactacgacgtggaccacatcgtgccgcagagcttcctcaaggacgacagcatcgacaacaaggtgctcacccggtccgacaagaatcggggcaagtccgacaacgtgcccagcgaggaggtcgtcaaaaagatgaaaaactactggcgacaactactgaacgccaagctcatcacccagcgcaagttcgacaacctcacaaaagccgagcgcggcgggttgagcgagctggacaaggccgggttcatcaagcgccagctcgtcgagacgcgccagatcacgaagcacgtcgcgcagatactcgacagccggatgaacaccaagtacgacgagaacgacaagctcatccgggaggtgaaggtcatcaccctcaagtcgaagctcgtgagcgacttccgcaaggacttccagttctacaaggtccgggagatcaacaactaccaccacgcccacgatgcttatcttaacgccgtggtggggacggccctcattaagaaatacccgaagctggagtcggagttcgtgtacggcgactacaaggtgtacgacgtcaggaagatgatcgccaagtccgaacaggagatcgggaaggccacggcgaaatacttcttctacagcaacatcatgaacttcttcaagaccgagatcaccctcgccaacggcgagatccgcaagcgcccgctcatcgagacgaacggggagaccggcgagatcgtctgggacaaggggcgcgacttcgccactgtgcggaaggtgctgtcgatgccccaggtcaacatcgtcaagaagacggaggtccagacgggcgggttcagcaaggagagcatcctgccgaagcgcaacagcgacaagctgatcgcccgcaaaaaggactgggatccaaaaaagtacggcggcttcgacagccccaccgtcgcctacagcgtcctcgtcgtcgctaaagtcgagaagggcaagtccaaaaagctcaagagcgtcaaggagctgctcgggatcaccatcatggagcggtccagcttcgagaagaacccaattgatttcctggaggcgaagggctacaaggaggtcaagaaagacctcatcataaagctgccgaagtactcactcttcgagctggagaacgggcgcaagcggatgctggcgtcggccggagagctccaaaagggcaacgagctggcgctgccgagcaagtacgtgaacttcctctacctggcgtcccactacgagaagctcaagggcagtccagaggataacgagcagaagcagctattcgtggagcagcacaagcactacctggacgagatcatcgagcagatcagcgagttctccaagcgcgtcatcctggcggacgccaacctggacaaggtgctgtccgcgtacaacaagcaccgcgacaagccgatccgcgagcaagccgagaacatcatccacctgttcaccctcacgaacctcggggcacccgccgccttcaaatatttcgacacgaccatcgaccgcaagcgctacaccagcacgaaggaggtgctcgacgccaccctgatccaccagagcatcaccgggctgtacgagacccgcatcgacctctcgcagctcggcggggac

SEQ ID NO: 65 - DNA, 4101 bp, synthetic construct
  misc_feature 1..4101: note = Synthetic polynucleotide
  source 1..4101: mol_type = other DNA
gacaagaagtacagtattggattggccatcgggacgaacagcgtgggctgggccgtcatcaccgacgagtacaaggtgccatccaagaagtttaaggttctggggaataccgaccgccactcgatcaagaaaaatctcatcggggcgctgcttttcgacagcggcgagacggcggaagcgacgcggctcaagcggacggctcgtcgccgttacacccggcgtaagaaccgcatctgttacctccaggagatattcagcaacgagatggcgaaggtggacgactcctttttccaccgtcttgaggagtccttcctggtcgaggaggacaagaagcacgagcgccacccgatcttcgggaacatcgtggacgaggtggcctaccacgagaagtaccccacgatctaccacctccgcaaaaaactcgtggactcaactgacaaggccgatttgaggcttatctacctcgccctcgcccacatgattaagttccgtgggcacttcctaatcgagggtgacctcaaccccgacaactctgacgtggacaagctgttcatccagcttgtgcagacctacaatcagctctttgaggagaatccgatcaacgcatctggtgtggacgcaaaggccatcctcagcgcgcggctgagcaagtctaggcggttggagaacctgatcgcccaactgcccggcgagaagaaaaatggcctcttcggcaacctgatcgccctgtcgctggggctcacgccgaacttcaagagtaactttgacctggcggaggacgctaagctccagctatctaaggacacatacgacgacgacctggacaacctgctggcccagatcggcgaccagtacgccgacctcttcctagccgccaagaacctgtccgacgccatcctcctcagcgacatcctgcgcgtgaacacggagatcacgaaggctccgctcagcgcctccatgattaagcggtacgacgagcaccaccaagacctaactttactcaaagccctcgtgcggcagcagcttcccgagaagtacaaagagatattttttgatcagtccaagaacggttatgcgggctacatcgacggcggcgcgagccaggaggagttctacaagttcatcaagcccatcctggagaagatggacggcacggaggagctgctcgtgaagctcaaccgtgaagacctcctgcgaaagcagcgaaccttcgacaacggttcgatcccgcaccagatccacctcggggagctgcacgccatcctgaggcgacaggaggacttctaccctttcctaaaggacaaccgcgagaagattgaaaaaatcctgacgtttcgcataccctactacgtcggcccgctggcgcgcggcaactcccggttcgcctggatgacccgtaagagcgaggagacgatcaccccgtggaacttcgaggaggtcgtggacaagggcgcgagcgcgcagagcttcatcgagcgcatgaccaacttcgacaagaacctcccgaacgagaaggtgctcccaaagcactccctcctgtacgagtatttcaccgtgtacaacgagttgacaaaggtgaagtacgtgacggagggaatgcggaagcctgcgttcctctcgggcgagcagaagaaggcaatcgtggacctgctcttcaagaccaaccggaaggtgacggtgaagcagctcaaggaggactacttcaaaaaaatcgagtgcttcgactccgtggagataagcggcgtggaggaccgattcaacgcctccctcggcacctaccacgacctccttaagatcatcaaggacaaggacttcctggacaacgaggagaacgaggacatcctggaggacatcgtgctcaccctgaccctcttcgaggaccgggagatgatcgaggagcgcctcaagacgtacgcccacttgttcgacgacaaggtgatgaagcagctcaagcggcggcgatacaccgggtggggccgcctatcccgcaaacttatcaacggcatccgcgacaagcagtccggcaagacgatcctggatttcctcaagtcggacgggttcgccaaccggaacttcatgcagctcatccacgacgacagcctcacgttcaaggaggacatccagaaggcccaagtgagcggtcaaggggacagcctccacgagcacattgcgaaccttgctgggagccctgcgatcaagaaggggatattgcaaaccgtgaaggtcgtggacgagttggtgaaggtcatggggcgacacaagcccgagaacatcgtgatcgagatggccagggaaaatcagaccacgcagaagggccaaaaaaacagccgcgagcggatgaagcggatcgaggagggcatcaaggagctggggtcgcagatcctcaaggagcacccggtggagaacacgcagctccagaacgagaagctgtacctctattacctacagaacgggcgggatatgtacgtggaccaggagctagacatcaaccgcctgtccgactacgacgtggaccatatcgtcccgcagtcgttcttgaaggacgacagcatcgacaacaaggtgctcacaagatcggataagaatcgaggcaagtccgacaacgtgccctcggaggaggtggtcaagaaaatgaaaaactactggcggcagttgctgaacgccaagctcattacgcagcggaagttcgacaacctgacgaaggctgaacgtggtgggctcagcgagctagacaaggcggggttcatcaagcggcagctcgtcgagacccggcagatcaccaagcacgtggcgcagatcctggactcgcgcatgaacaccaagtacgacgagaacgacaagctcatccgtgaggtgaaggtcatcacccttaagtctaagctggtcagtgacttccgcaaggacttccagttctacaaggtccgggagatcaacaactaccaccacgcgcacgacgcctacctcaacgcggtggtggggacggcgcttattaagaaatatcccaagctggaaagcgagttcgtttacggcgactacaaggtgtacgacgtccgcaagatgatcgcaaagtcggaacaggaaatcggaaaggcgacggccaaatatttcttttactccaacatcatgaatttttttaagacggagatcaccctggcgaacggggagatccgcaagcggcccctcatcgagaccaacggggagacgggcgagatcgtctgggacaagggccgggacttcgccaccgtgcggaaggtgctttctatgcctcaagtcaatatcgtcaaaaagacagaggtgcagaccggcgggttcagcaaggagtctatcctgccgaagcgcaactcggacaagctcatcgcgcgcaagaaagactgggaccccaaaaaatatggcgggttcgactcgccgaccgtcgcctacagcgtcctcgtggtggctaaggtcgagaagggcaagagcaaaaagctaaagtcggtgaaggagctgctgggcatcaccatcatggagcgctcgtctttcgagaagaatccaatcgacttcctagaggcgaaggggtacaaggaggtcaaaaaggatcttatcatcaaactgccgaagtacagtctgttcgagctggagaacgggcggaagcggatgctggctagtgcgggcgagttgcagaagggcaacgagttggcactgccctccaagtacgtgaacttcctgtacctggcctcccactacgagaagctcaaggggagccccgaggacaacgagcagaagcagctattcgtcgagcagcacaagcactacctggacgagatcatcgagcagatcagtgagttctccaagcgggtcatcctcgcggacgccaacctggacaaggtgctgagcgcgtacaacaagcacagggacaagccaatcagggaacaggccgagaacatcatccacctgttcaccctgaccaacctgggtgcaccggctgccttcaagtactttgacacgaccatcgaccggaagcgctacacctccacgaaggaggtgctggacgccacgctgatccaccagagcatcaccgggctctacgagacacggatcgacctgagccagcttggcggggac

SEQ ID NO: 66 - DNA, 4092 bp, synthetic construct
  misc_feature 1..4092: note = Synthetic polynucleotide
  source 1..4092: mol_type = other DNA
gacaaaaagtattccattggactcgctatcggcacgaacagcgtcgggtgggcggtcatcactgacgagtacaaggtgccgagcaagaagtttaaggtgctgggaaacaccgacaggcactcgatcaagaaaaatcttatcggggccctactcttcgactccggagaaaccgccgaggccacccggttgaagcgcacggcccgccgtcgctacaccaggcgcaagaaccggatctgctacctccaggagatattcagcaatgagatggcgaaggtggacgactcgttttttcacaggctagaggagtctttcctcgtggaggaggacaagaaacacgagcgccaccccatcttcggcaacatcgtggatgaggtggcatatcacgagaagtacccaaccatctaccacctccgcaaaaagctcgtggactctaccgacaaggccgacctccgtctgatctacctcgcgctggcccacatgattaagttccgaggacactttctgatcgagggcgacctgaacccagacaacagcgacgtggacaagctgttcatccaacttgtccagacctacaatcagctcttcgaggagaaccctatcaacgcctcgggcgtggacgcgaaggccatcctgtccgcccgcctgagcaagtcgcggcggctggagaacctgatcgcccagctccccggcgaaaaaaagaacggcctcttcggcaacctcatcgcgttgtcgctggggctcaccccgaacttcaagtccaacttcgacctggccgaggacgctaaactccagctctcgaaggatacctacgacgacgacctcgacaacctgctggcccagatcggcgaccagtacgcggaccttttcctggcggccaagaacctgagcgacgcgatcctccttagcgacatactccgtgtgaacaccgagatcacgaaggccccgctctccgcgtccatgattaagcgctacgacgagcaccaccaagaccttaccctgcttaaggcgctggtcaggcagcagttaccggagaagtacaaggagatcttttttgatcaatctaagaacggttacgccgggtacatcgacggcggcgcgtcccaggaggagttctacaagttcatcaagccgatcttggagaaaatggacgggaccgaggagctgctcgtgaagctcaaccgcgaagacctcctccgcaagcagcgcaccttcgacaacgggagcatcccgcaccagatccacctgggagagctgcacgcgatcctgcggagacaagaggacttctaccccttcctcaaggacaaccgggagaagattgaaaaaatacttacttttcgtatcccgtactacgtcgggccccttgcgaggggcaactccagattcgcgtggatgacccgcaagtccgaggagaccatcaccccgtggaacttcgaggaggtggtggacaagggcgcgtcggcccagtcgttcatcgagcgcatgaccaacttcgacaagaaccttccgaacgagaaggtgctcccgaagcacagcctgctctacgaatattttactgtgtacaacgagctgacgaaggtcaagtacgttacggaggggatgaggaagcccgccttcctctccggcgagcagaagaaagccattgtggatctcctgttcaagaccaaccgcaaggtgacggtgaaacagctcaaagaggactacttcaagaagatcgagtgcttcgactccgtagagatcagcggggtcgaggaccgcttcaacgcctcgctgggcacgtaccacgacctgctaaagattatcaaggacaaagacttcctagacaatgaggagaacgaggacattctggaggacatcgtgctgactctgacgctgttcgaagaccgcgagatgatcgaggagcggcttaagacgtacgcccacctgttcgacgacaaggtgatgaagcagttgaaacggcggcgctacaccgggtggggccgcctctcccgcaagctcatcaacggcatccgcgacaagcagtcggggaagacgatcctggacttcctcaagagcgacggcttcgccaaccgaaacttcatgcagctaatccacgacgacagcctgacgttcaaggaggacatccagaaggcccaagtgagcggccagggagactcgctacacgagcatatcgccaacctggctggcagcccggcgattaagaaaggaatcctccaaaccgtcaaagtggtggacgagctggtgaaggtgatgggccgccacaagcccgagaacattgtgatcgagatggcgcgggagaaccagacgacgcagaagggccaaaaaaatagcagggaaaggatgaagcgaatagaggaggggatcaaggagctggggagccagattctcaaagagcacccggtcgagaacacacagctccagaacgagaagctgtacctctactacctccaaaacggccgcgatatgtacgtggaccaggaactagacatcaaccggctgagcgactatgacgtggaccacatcgtgccgcagtccttcctcaaggacgactcgattgacaacaaagtgctcactagatccgacaagaacagaggcaagagcgataacgtcccgtcggaggaggtcgtcaagaaaatgaaaaactactggcggcagctcctaaacgccaagctcatcacgcagcgtaagttcgacaacctgacgaaggcggagcggggcgggctgagcgagctggacaaagcggggttcatcaagcggcagctcgttgagacgcggcagatcacaaagcacgtcgcgcaaatcctcgactcccgcatgaacaccaagtacgacgagaacgacaagctcatccgggaggtgaaggtcattacccttaaatcgaagctcgtcagcgactttcgtaaggacttccagttctacaaggtcagagagatcaacaactaccaccacgcccacgacgcctatctgaacgccgtggtgggcaccgcgcttattaagaagtaccccaagctggagtccgagttcgtgtacggcgactacaaggtttatgacgtcaggaagatgatcgccaagtcggaacaggagatcggaaaagctaccgccaaatatttcttctatagcaacatcatgaacttcttcaaaaccgagatcaccctcgccaacggcgagatccggaagcgcccgctcatcgagaccaacggggagaccggggagatcgtctgggacaaggggcgggacttcgctactgtccgaaaggtgctctccatgccacaagtgaatatcgtcaagaaaacagaggtgcagaccggagggttcagtaaggagtccatcctgcccaagcggaactccgacaagctaattgctcgcaaaaaggattgggatcctaaaaaatatggcggcttcgactcgcccacggtcgcctactctgtgctggtcgtggcgaaggtggagaagggcaagtccaagaagctcaagagcgtcaaggagctgctggggatcacgatcatggagcgtagttcgtttgagaagaatcccatcgacttcctggaggctaagggctacaaggaggtcaaaaaggacctcatcattaagctgccgaagtacagcctcttcgagctggagaacgggcggaagcgtatgctcgcctccgctggggagttacaaaaggggaacgagctggcgctgccgtctaagtacgtcaacttcctgtacctggcctcccactacgagaagctcaaggggtcgccggaggacaacgagcagaagcagctcttcgtagagcagcacaagcactacctggacgagatcatcgagcagatttcagagttctcaaagcgggtcatcctcgccgacgccaacctggacaaggtgctctcggcctacaacaagcaccgggacaagccgatccgcgaacaggccgaaaacatcatccacctgttcacgctcaccaacctcggtgccccggcggccttcaagtactttgacacgaccatcgaccggaagcgctatacctcgacgaaggaggtgctggacgccaccctgatccaccagtccatcaccgggctttacgagacccggatcgacctctcgcagcta

SEQ ID NO: 67 - DNA, 4101 bp, synthetic construct
  misc_feature 1..4101: note = Synthetic polynucleotide
  source 1..4101: mol_type = other DNA
gacaagaagtatagtattggactcgccatcggaaccaactctgtggggtgggctgttattacagatgaatataaggtgccatccaaaaagtttaaagttctgggcaatactgatagacactcaatcaagaagaatctgataggtgcacttctgtttgatagtggagagactgccgaggcaaccagacttaaaaggactgcaagaagaagatataccagaagaaagaataggatttgctatttgcaggaaatcttcagcaacgaaatggccaaggttgatgactcatttttccataggttggaggagagttttcttgtggaggaagataagaagcacgaaagacacccaattttcgggaatatagtggacgaggtggcttatcatgagaagtatcccactatctaccacctgagaaagaaacttgtggactcaaccgataaggctgatcttaggcttatatacttggcccttgcacatatgatcaaattcaggggccattttcttatcgaaggcgatcttaatcccgataactcagatgtggacaagctgtttatacaacttgtgcaaacctacaatcaactcttcgaggagaatcccattaacgcctccggcgtggatgcaaaagccatactgtcagccagactgagcaaaagtaggagactggagaatcttatagcccaactgcccggtgaaaagaagaatgggctcttcggaaatctgatcgctctttcattggggttgacacccaactttaagagtaactttgacttggcagaagatgcaaagttgcagctcagtaaagacacatatgacgatgaccttgacaatctcttggcacaaataggggatcaatacgctgaccttttcctcgctgccaagaacctcagcgacgctatactgttgtccgacattcttagggttaataccgaaattacaaaggcccctcttagtgcaagtatgatcaaaaggtatgatgagcatcaccaagaccttacactgctgaaggctctggttagacagcaactccctgaaaagtataaggaaatattcttcgaccaaagtaagaacgggtacgccggttatattgatgggggcgcaagtcaagaagaattttacaaattcatcaagccaattcttgaaaagatggacgggactgaggaattgctggtgaaactgaatagagaggaccttcttagaaaacagaggacatttgacaatgggtccatcccacaccagattcatctgggggaactccacgcaatattgaggagacaagaagacttttacccattccttaaggataatagagagaaaatcgaaaaaatcctgactttcaggattccttactatgttgggccactggccagggggaactcaagattcgcttggatgacaaggaagtcagaagaaaccataaccccttggaattttgaagaggtggttgataagggggcatcagcccagtctttcatagagaggatgaccaactttgataaaaatcttccaaatgagaaggttttgccaaaacatagtcttttgtacgagtactttactgtttataacgaattgaccaaggtgaagtatgtgaccgagggaatgaggaagccagcatttttgtccggggagcaaaagaaagcaatcgttgatcttctcttcaagaccaacagaaaagtgaccgtgaaacaactgaaggaagactacttcaaaaagatagaatgtttcgattcagtggaaattagcggtgttgaagacaggttcaatgcttcattgggtacttaccacgacctgttgaagataatcaaagacaaggactttctcgataatgaggagaacgaagacatcttggaagacattgtgcttacactcactttgtttgaggacagggaaatgattgaggaaagactcaaaacttacgctcatttgtttgatgataaggttatgaaacaactaaaaagaagaaggtacaccggctggggaagattgagtaggaaactgatcaacggtattagagataaacaatccggaaagactatcctcgatttccttaagagtgatggctttgcaaataggaattttatgcagctgattcatgacgactcacttaccttcaaagaagacatccaaaaagctcaggtgtctgggcaaggcgacagtctgcatgaacatatagctaacttggctgggagtcccgccatcaagaaggggatacttcaaacagttaaagttgtggacgaattggtgaaggtaatgggaaggcacaagcctgaaaatatagtgatagaaatggcaagggaaaatcaaacaacccagaagggacagaagaacagtagggaaaggatgaaaaggatagaagaggggatcaaagagcttggtagccagatcctcaaggaacatccagtggagaatacccaacttcaaaacgagaaactctatttgtactacttgcagaacggaagagatatgtatgtggaccaagagcttgatattaacaggctgagcgattatgacgttgaccacatagtgccccaatcattcctcaaggatgactctattgataataaggtgctgacaaggagtgacaagaatagagggaaatccgacaacgttccatccgaggaagttgtgaagaagatgaagaactactggaggcagttgctgaacgctaagctcattacccagaggaaattcgataacctgaccaaagcagagagaggcgggctgagcgaactcgataaagcaggtttcatcaagagacaactcgtggagactaggcaaattactaagcacgtggctcaaatactcgacagcaggatgaacacaaagtacgacgagaacgacaagctcattagagaggttaaggttattactctgaaaagtaaattggttagcgatttcagaaaggatttccaattctataaggttagagagatcaacaattatcatcatgcacatgatgcctatctgaatgctgtggttggtacagcccttatcaagaagtaccctaagctagagagcgagtttgtgtacggagattataaggtgtatgatgtgaggaaaatgatcgctaaaagtgagcaagagattggaaaggctaccgccaaatacttcttttattccaatattatgaatttcttcaagacagaaatcaccctggctaacggcgagataaggaagaggccgcttatcgaaactaatggggagacaggcgaaatagtgtgggacaaagggagggatttcgcaactgtgaggaaggttttgagcatgcctcaggtgaatatcgttaagaaaaccgaagttcaaactggagggttctctaaggaaagcattctccccaagaggaactccgacaagctgattgctagaaagaaagactgggaccccaagaagtatggcggattcgactcacccactgtggcatatagcgttctcgtggtggcaaaggttgaaaagggtaaatccaaaaaactcaaatccgtgaaggaactccttggcataactattatggaaaggagtagctttgaaaagaatcccatcgactttctcgaagctaagggctataaggaagttaagaaggaccttataatcaaacttccaaaatactccctttttgagttggaaaacggcagaaagagaatgttggccagtgccggggagcttcaaaagggcaacgaactggctctgcctagcaaatatgtgaactttttgtatctggcatcacactacgagaaacttaaaggctctcctgaggacaacgagcaaaaacagctctttgttgaacagcataagcactacctcgacgagattattgagcagatcagcgagttctcaaagagagttattctggctgacgctaatcttgacaaggttttgtccgcttacaacaaacacagggataagccaatcagggagcaggcagaaaacataatccatctctttaccctgacaaacctcggtgcccccgctgctttcaagtattttgatactaccattgacaggaagagatatacttccactaaggaagtgctcgacgcaaccctcatacaccaaagtatcacaggcctctatgaaactaggatagatttgtctcaacttgggggcgat

SEQ ID NO: 68 - DNA, 4101 bp, synthetic construct
  misc_feature 1..4101: note = Synthetic polynucleotide
  source 1..4101: mol_type = other DNA
gacaaaaagtattccatcgggcttgctatcggaaccaactctgtggggtgggcagttattaccgacgaatacaaggtgcccagcaagaagtttaaggttctggggaacacagatagacatagcataaagaaaaacctgataggcgcactgttgttcgactccggggaaacagccgaagctaccaggctgaagagaactgcaagaagaaggtacaccagaagaaaaaacagaatatgttatctccaagagattttctctaacgagatggccaaggtggacgactcattctttcacagactggaagaatctttccttgtggaagaagataagaaacacgagaggcaccctatttttggcaatatcgtggatgaggtggcttaccacgaaaaataccctacaatataccacctcaggaaaaaattggttgatagtacagacaaggccgacctcaggctcatctatttggccctggcccatatgattaaattcagggggcactttctcatcgagggagatttgaaccccgacaacagtgatgttgataagctctttattcagctcgtgcagacttacaatcagttgtttgaggaaaaccccattaatgcttccggggtggacgccaaggcaatcctttctgcaagactctcaaagtcaaggagactcgaaaatctgatagcacagcttccaggagagaagaagaacgggctctttggaaacctgatcgctctgtcactcggactcacacccaatttcaaaagcaattttgatttggcagaggacgctaagctgcaactcagtaaggatacctacgacgatgacttggataatctgctcgcacaaattggggaccagtatgcagacctgtttctcgcagctaagaacttgagtgacgccatattgctcagtgacatcctcagggttaataccgagattacaaaagctccactctctgcaagcatgatcaagaggtatgacgagcaccatcaagacctgacactccttaaggcgttggttaggcagcaacttcctgaaaagtataaggaaatcttcttcgatcaaagcaaaaacggctacgccggctatatagacgggggagcatcccaagaagaattttataagttcataaaacctatattggagaagatggacgggacagaggaattgctcgtgaaactgaacagggaggatctcctcaggaagcaaaggaccttcgacaatggctccatcccacatcagattcacctcggcgaactgcacgcaatactgagaagacaagaggacttttatcctttcctgaaggacaacagggagaaaatcgagaaaatcttgacattcagaatcccatactacgttgggcctctggccagaggtaacagtaggttcgcctggatgactaggaaatcagaggagactattacaccctggaactttgaagaagttgttgataagggagcttcagcacaatcattcatcgaaagaatgacaaactttgacaaaaatctgcctaatgagaaagtgctcccaaaacattccctgctgtatgagtattttaccgtttataacgagcttaccaaggtgaaatacgttactgaaggtatgagaaagccagcttttctttcaggggagcaaaagaaggctatcgtggatcttctctttaagaccaacagaaaggttaccgtgaagcagcttaaggaagactactttaaaaagatcgagtgttttgactcagtggaaataagcggtgttgaagatagattcaacgcatccttgggaacttatcatgatcttcttaagataatcaaggataaagactttctcgacaacgaggaaaacgaagatatactggaggacatagttctgacacttactttgttcgaggatagggagatgatcgaggaaagactgaaaacatatgctcaccttttcgacgacaaagttatgaaacaactcaagagaaggagatatacagggtgggggagattgagcaggaaactgattaatggtatcagagacaaacagtcaggaaaaacaatactcgactttttgaaatcagacgggttcgcaaataggaatttcatgcagcttatacacgacgattcacttacttttaaagaggacattcaaaaggctcaagttagtggacaaggtgactccctccacgaacacatcgcaaatctcgctggcagccctgcaattaagaagggtatactccagacagttaaggttgttgacgagctggttaaagtgatgggaagacacaaacccgagaacatagtgatagagatggccagggaaaaccaaaccactcaaaaagggcagaaaaattccagagagaggatgaaaaggattgaagaaggtatcaaggagctgggtagccaaattctgaaagaacatcctgtggaaaacactcaactccagaatgagaaactctatctgtactatctgcaaaatgggagagatatgtatgtggaccaggaactggacataaacaggctctcagattacgatgtggatcatatcgtgccacagtcctttcttaaggatgatagcatcgacaataaggtgcttaccaggtccgacaagaacaggggaaagtcagataacgtgccttctgaagaagttgttaaaaagatgaagaactactggagacagctgcttaacgctaagctcataacacagaggaagtttgacaacttgaccaaggccgagagaggcggactctcagaattggataaggcagggttcataaaaaggcagctggtggaaacaaggcagataactaaacatgtggctcagatcctcgatagtaggatgaatacaaaatacgatgagaacgacaagctcataagggaggttaaagtgataactctgaaatccaaactggttagcgattttaggaaggatttccagttttacaaagttagggagatcaacaattatcatcacgcccacgatgcctacttgaacgcagttgtgggtactgcacttatcaaaaagtaccctaagctggaatccgagtttgtttatggagactataaggtgtacgacgttagaaaaatgattgcaaagtcagagcaggagatagggaaagccactgcaaaatatttcttttatagcaatatcatgaatttctttaagacagaaatcacactggccaatggggaaataaggaagaggcccctgatcgaaactaatggcgagacaggggagattgtgtgggataaaggtagggactttgcaacagtgaggaaagtgctgagcatgccccaagttaatatcgttaaaaagaccgaggttcaaacagggggctttagtaaggaaagcattttgcccaagaggaatagtgacaaattgattgctaggaaaaaagattgggaccccaaaaagtatggcggatttgatagccccactgttgcttactccgtgctcgtggttgcaaaggtggagaagggaaagagcaagaaactgaagtcagttaaggaactccttggtatcactatcatggaaagaagctcctttgagaagaaccctattgacttcctggaggctaaagggtacaaagaggttaagaaagaccttatcattaaattgcccaaatatagtcttttcgagcttgaaaacggaagaaagaggatgcttgcatccgctggcgaattgcaaaagggcaatgagcttgctctcccttccaagtatgtgaacttcctttatcttgcctcacactatgaaaaactcaaaggttcacccgaagacaacgaacaaaagcaactatttgtggaacaacacaagcactacctggacgaaatcattgagcaaatttctgagttttcaaaaagggtaatcttggctgacgcaaatctcgacaaagttttgtcagcttacaacaaacatagagataagccaattagagagcaagctgagaatatcatccatctgtttaccctgactaaccttggagcgcctgctgcttttaaatatttcgacaccacaatcgacaggaagaggtacactagcactaaggaagttctcgacgccaccctcatccaccagagtattacaggcctgtacgagacaagaattgatctttctcaacttggtggtgac

SEQ ID NO: 69 - DNA, 4101 bp, synthetic construct
  misc_feature 1..4101: note = Synthetic polynucleotide
  source 1..4101: mol_type = other DNA
gataagaagtactcaatcggtctggcaatcggaaccaactctgtgggttgggcagtgattacagatgagtataaggtgccaagcaaaaaattcaaggtgctgggtaataccgacagacacagcattaagaagaatttgattggagcactcctctttgactcaggggaaacagcagaggcaacaaggctgaagaggacagcaaggcggaggtacacaaggcggaaaaacaggatatgctacctccaggaaatctttagcaacgagatggctaaagtggatgatagctttttccatagactcgaagaatcctttcttgttgaagaggacaaaaagcatgaaaggcatcccatcttcggcaatatagttgatgaggttgcataccatgagaagtaccccacaatctaccacctcagaaagaaacttgtggactccacagataaagcagacctgaggctcatatacctcgcactcgcacacatgatcaagttcagagggcactttctcatcgaaggtgacctgaatccagataattcagatgtggataaactgtttatacagctggtgcaaacatacaaccaacttttcgaggaaaacccaatcaatgcctccggtgttgatgcaaaggccatcctgtcagcaagactcagcaaaagcaggcggctcgaaaacctcatcgcccagcttcccggtgaaaagaagaacgggctctttggtaatctcatcgcattgagccttggtcttactccaaacttcaagagcaattttgatctggcagaggatgctaaactgcaactctcaaaggacacatatgacgatgaccttgacaatctgttggcccagatcggggaccaatatgcagacctcttcctggccgcaaagaatctgtcagatgcaatcctcttgtccgacatactgagagttaacactgagatcacaaaggcacctctgtccgcctccatgattaagagatacgatgagcatcaccaggatctgactttgctcaaagccctcgttagacagcagttgccagaaaagtacaaagaaatattctttgatcaatcaaaaaacggatatgcagggtacatcgacggtggggcaagccaggaagagttctacaaattcatcaaacctatcctggaaaagatggatgggacagaagagctgctggttaagctgaatagggaagacctcctcagaaagcagaggacatttgataacgggagcatccctcatcaaatccacctcggtgaactccatgctatcctgagaaggcaggaagacttttatccatttttgaaggacaatagggagaaaatcgaaaaaatcctgacattcagaatcccatactacgttggtcctctggcaagaggtaacagtaggttcgcatggatgacaaggaaaagcgaggagacaatcacaccctggaattttgaggaagttgttgacaagggtgccagcgcacaatcctttatcgaaagaatgacaaatttcgacaagaatctgcctaacgaaaaggttctcccaaagcattcactcctgtacgaatattttacagtttataacgaactgactaaagttaaatacgttaccgagggtatgaggaagccagcattcctttccggggaacagaagaaagctattgtggacctcctgttcaagacaaatagaaaagtgacagttaagcaactcaaagaggattacttcaaaaagatcgaatgttttgactctgtggagatcagcggggtggaggatagattcaacgccagcctgggtacatatcatgatctcctgaaaatcattaaagacaaggacttccttgacaacgaggagaacgaggacattctggaagacattgttctgaccctcacactctttgaggatagggagatgattgaggaaagactgaagacctacgcccacctctttgacgataaagtgatgaaacagctcaagagaagaaggtatacaggttgggggagactgagcaggaagttgatcaatgggattagggacaaacagtccgggaaaacaatcctcgattttctgaagtcagacggtttcgcaaacagaaattttatgcagctcattcacgatgacagcttgacattcaaggaagacatccaaaaggctcaagtgagcggccaaggggatagcctccacgagcatattgcaaatctggcaggttcaccagccatcaaaaagggcatacttcagacagttaaggttgtggacgaattggttaaagttatgggcaggcataagccagagaatatcgttatcgaaatggcaagggagaaccaaacaactcaaaaagggcagaaaaatagcagagagaggatgaaaagaatcgaggaagggatcaaggaacttgggtcccaaatcctcaaggagcacccagttgaaaatactcaactgcaaaacgagaagctctatctctactatctccaaaacgggagggatatgtatgttgaccaggagctggatattaacagactgtcagattatgatgttgatcatatcgtgccccagtcattcctgaaggacgattccatcgacaacaaagttctcacaaggtccgataaaaacaggggcaagtccgataacgttccaagcgaagaagtggtgaaaaagatgaaaaactattggagacaacttctgaatgcaaagttgattactcagagaaagtttgacaacctcacaaaagcagaaagaggcgggcttagcgaactcgataaggcagggtttatcaaaagacagctggttgagacaaggcagatcacaaaacatgtggcacagatccttgactcaaggatgaataccaagtatgatgagaatgataagttgatcagggaggttaaagttatcacactcaaatccaaactggtgtcagacttcaggaaagactttcaattttataaggtgagggagatcaataactaccaccatgcacatgacgcctacctgaacgcagtggtgggtacagcattgattaaaaaataccctaagctggagtctgagtttgtgtacggggactacaaggtgtacgacgtgaggaaaatgatagccaagtccgagcaggagatcgggaaagcaacagctaagtatttcttttacagtaatatcatgaatttctttaaaactgagattactctggcaaacggggagatcaggaaaagacccctcatcgagactaatggtgaaacaggtgagatcgtttgggacaaggggagggattttgctactgttagaaaagttctgagtatgccacaagtgaatattgtgaaaaagacagaagttcagacaggtgggttctccaaagaatccatcctgcccaagagaaattcagacaagctcatcgcaagaaagaaggactgggaccctaagaagtacggaggatttgacagccccaccgtggcctattccgtgcttgttgtggcaaaggtggagaaagggaagagcaaaaaactgaaatccgtgaaagaactgctgggaattaccatcatggaaagaagctcctttgagaagaacccaatcgacttcctggaagcaaaaggatataaggaagtgaaaaaggacctcattatcaagctcccaaaatactcacttttcgagttggagaacggtagaaagaggatgctggcaagcgcaggggaacttcagaaaggcaatgagctggcattgccatcaaagtatgtgaacttcctctacttggccagccattacgagaaacttaaaggtagcccagaagataacgagcaaaaacagctctttgtggaacagcataagcattatctggatgagatcatagaacaaatctcagagttttccaagagagttatcctcgcagatgcaaacctggataaggttctctcagcctataataagcatagagacaagccaattagagagcaagcagagaacattatccacttgttcactcttacaaacctgggggcaccagccgccttcaaatatttcgatacaacaatagacagaaagaggtataccagcaccaaagaagttctcgacgccacactgatccatcaatcaatcacaggcctttacgaaactaggatcgacttgtcacaactgggtggggat

SEQ ID NO: 70 - DNA, 3307 bp, synthetic construct
  misc_feature 1..3307: note = Synthetic polynucleotide
  source 1..3307: mol_type = other DNA
gagcaaggacacctacgacgacgacttggacaacctattggcccagataggtgaccagtatgcagacctcttccttgcggccaagaacttgagtgacgctatactgctcagtgacatcctgagggtgaacactgagatcactaaggcccctctctctgcctcaatgattaagcgttacgacgagcatcaccaggatctcaccctgcttaaggcccttgttcggcagcagctccctgagaagtacaaggagatattttttgaccagtctaagaacggctacgccggttacattgacggtggggcaagccaggaggagttctacaagttcatcaagccgatccttgagaagatggacggcaccgaggagctacttgtcaagttgaaccgggaagacctgctccggaaacagcgtacattcgacaacggcagcatccctcaccagatccacctgggcgaactacacgccatcctccgacgtcaggaggacttctatccattcttgaaagataacagggaaaaaatcgaaaaaatacttacgtttcgaataccttactacgtggggccccttgctcggggaaactccagattcgcatggatgaccaggaagtcagaggagaccatcacaccctggaactttgaggaggtggttgacaaaggtgcttctgcccagtccttcattgagcggatgactaacttcgacaagaacctgcccaacgagaaggtgctgccaaagcacagcctgctctacgaatactttactgtgtacaatgagctgacgaaggtgaagtacgtgacagaggggatgcggaagcccgctttcctgagcggcgagcaaaaaaaagcaatcgtggacctactgttcaagaccaaccgaaaggtgacagtgaagcagctcaaggaggactacttcaaaaaaatcgagtgcttcgactctgttgagataagcggcgtggaggaccgattcaacgcctcattgggaacctatcacgacctgctcaagatcattaaggacaaggacttcctggataatgaggagaatgaggacatcctggaggatattgtgctgacccttactctattcgaggacagggagatgatcgaggagcgactcaagacctacgctcacctgttcgacgacaaggttatgaagcaattgaagcgtaggcgatacacggggtggggaagactctcccgaaaactgataaacggcatcagggacaagcagtcagggaagacgatcttggacttcctgaaatccgacgggttcgccaaccgcaacttcatgcagctcattcacgacgactcactaacgttcaaagaggacattcagaaggctcaagtcagtggacaaggcgactccctgcacgagcacattgcaaaccttgcgggctccccggcgattaaaaagggcattctccaaacggttaaggtggtggacgagctggtgaaggtgatgggccgacacaagcctgagaacatcgtgatcgagatggccagggagaaccagactacccagaagggtcagaagaactctcgggaacgtatgaagcgtattgaggaggggattaaggagttgggctctcaaatcctcaaggagcaccctgtggagaacactcagctccaaaacgagaagctgtacctgtactacctgcaaaacgggcgcgatatgtacgtggatcaggagttggacatcaacaggcttagcgattacgacgtggaccacatcgtgccacagtcattcttaaaggacgacagcatcgacaacaaggttctgacgaggagcgacaagaatcgagggaaaagtgacaatgttccatccgaggaggtggtcaagaaaatgaagaactattggcgtcagcttctgaacgccaagctcatcacccagcggaaattcgacaacctgactaaggctgagcgaggcggactctccgagcttgacaaggctggcttcatcaagcggcagttggtcgaaacccgacagataacgaagcacgttgcccagatacttgactcccgtatgaacaccaagtacgacgagaacgacaagctcatcagggaggtgaaggtcattacccttaagtccaaactcgtcagcgactttcgtaaggacttccagttctacaaggtgcgcgagatcaataactaccaccacgcacacgacgcctacctgaacgcagtggttggaaccgcgttgattaaaaagtaccccaagttggagtcggagttcgtttacggggactacaaggtgtacgacgttcggaagatgatcgccaagtctgaacaggagatcgggaaagcaaccgccaagtatttcttctatagcaacatcatgaacttctttaaaaccgagatcacacttgccaatggcgagatccgtaagaggccgctgatcgagacaaatggggagactggcgagatcgtgtgggacaagggccgcgacttcgcaaccgttcggaaagtcttgtccatgcctcaagtcaacatcgtcaagaagactgaggtgcaaacaggcgggttctcgaaggagtccatactgcccaagaggaactcagacaagctcatagcacgcaaaaaagactgggatccaaagaaatacggcgggttcgactcgccgacagtcgcatactccgtgttagtggtggctaaagtggaaaaggggaagtccaagaagctcaagtccgtcaaggagttgctcgggatcaccattatggaacggtcctcattcgagaagaatcccattgacttcctagaggcgaagggctacaaagaggtcaaaaaggacctaattattaagctccccaagtattcactcttcgaacttgaaaatggtcgtaagcggatgttggcaagcgctggagagcttcagaaggggaacgagcttgcactgccttccaagtacgtgaacttcctgtacctcgcctctcattacgagaagttgaagggctcaccggaggacaacgagcagaagcagttgttcgtggagcagcacaagcactacctcgacgagatcattgagcagataagtgagttcagcaaacgggtgatccttgccgacgctaacctggacaaggtgctgagcgcctacaacaagcacagagacaagccgatccgagagcaagcggagaacatcatacacctgttcaccctcacgaacctcggggctcccgcagccttcaaatattttgacacgaccatcgaccgtaaacgctacactagcacgaaggaggtgctggacgctacccttatccaccagtccatcaccggcctgtacgagacgagaatcgacttgtcgcagctcggtggtgac

SEQ ID NO: 71 - DNA, 4101 bp, synthetic construct
  misc_feature 1..4101: note = Synthetic polynucleotide
  source 1..4101: mol_type = other DNA
gacaaaaaatactcaattggtctggcaattgggaccaacagtgtcggatgggccgtgattaccgacgagtacaaggtgccgtccaaaaaattcaaggtgcttgggaacaccgaccgccactcgatcaagaaaaacctaatcggtgcgttgcttttcgacagtggggagaccgccgaggcaacacgcttaaaacgcacagctaggaggagatatacacggcgcaagaaccgaatatgctacttacaggagatattctccaatgagatggcgaaggtggacgactctttcttccatcggcttgaggaatccttcctggtcgaggaggacaagaagcacgagcgacacccgatattcgggaacatcgttgatgaggtggcgtaccacgagaagtacccaacgatataccacttacgcaagaagctcgtggactctacggacaaggccgacttgcgccttatctacttggcactggcccacatgattaagttccgaggccacttccttatcgagggtgacctgaaccccgataactccgacgtggacaagctcttcatccaactcgtccagacatacaaccagctattcgaggagaatcctatcaacgcctctggggtggacgctaaagctatcctctcagcccgcctgtcaaagtcgaggaggttggagaacctaatcgcccagcttccaggcgagaagaaaaatgggctgttcggaaaccttatcgcactctcactgggcctaaccccgaacttcaagtccaacttcgacctggcagaggacgcgaaattgcagttgtcgaaagacacctatgacgatgacctggacaacctgttggcccagataggggaccagtacgccgacctgttcctagcggccaagaacctgtccgacgccatcttgctgtcggatatactgcgggtgaacaccgagatcactaaagcacctctctccgccagcatgattaagcgttacgacgagcaccaccaagatttgaccctgctaaaggcacttgtacggcagcagcttcccgagaagtacaaggagatctttttcgaccaaagcaagaacggctacgccgggtacatcgacggaggtgccagccaggaggagttctacaagttcattaagcccatcctggagaagatggacgggactgaggaactacttgtgaagctgaaccgggaagacttactacggaagcagcgtaccttcgacaacggttctatcccacatcagatccatcttggggagttgcacgcgatcctgcgacgccaggaggacttttaccccttcctgaaagacaaccgcgagaaaatcgagaagatactgaccttcagaataccttactacgtcggaccccttgcgcgaggcaactcaagattcgcgtggatgaccaggaaatcagaggagaccatcacaccctggaatttcgaggaggtggttgacaagggtgcctccgcccagtcctttatcgaacgaatgaccaacttcgacaagaacttgcccaacgagaaggtgctccccaaacacagcctcctctacgaatatttcacagtgtacaacgagcttactaaagttaagtatgttactgagggcatgaggaaacccgccttcctgtcaggcgagcagaagaaagctattgtggacctccttttcaagaccaaccggaaggtgacagtgaagcagctcaaggaggactacttcaagaagatagagtgcttcgacagcgtggagatcagcggggtggaggacagattcaatgcctctctcggaacataccacgacttgcttaagatcatcaaggacaaggacttcctcgacaacgaggaaaacgaggatattctggaggatattgttctgactcttaccctgttcgaggaccgggagatgatcgaggagcgtctcaagacctacgcccacctgttcgacgacaaagttatgaagcagctcaagcgtcggagatataccggatggggccgtctgtctcggaagctcatcaacgggatcagggacaagcagtcagggaagacgatcttagacttccttaagtctgacggcttcgccaacaggaacttcatgcagttgatccacgacgacagccttaccttcaaggaggacatccagaaggcccaagtgagtggccagggtgacagcctccacgagcatattgctaatcttgcgggttccccagcgattaaaaagggcatacttcaaaccgttaaggtggtggacgagcttgtcaaggtgatggggcgacacaagcccgagaacatcgtgatcgagatggccagggagaaccagaccacccagaaggggcagaagaatagccgagaacgcatgaagcgcatcgaggaggggattaaggagctagggagccagatcctcaaggaacatcccgtcgagaacacccagctccagaacgagaagctatacctctactacttgcaaaacgggagggatatgtacgtggatcaggagttggacattaaccgcctaagcgactacgacgtagatcacatcgtgcctcagtcattcctcaaagacgacagcattgacaacaaagtcttgacccgatccgacaagaaccgaggaaaatccgacaatgtgccctcagaggaggtcgtcaagaaaatgaagaactattggaggcagctacttaacgccaaactcataacccagcggaagttcgacaacctgacaaaggctgagcggggtgggctcagcgagcttgacaaggctggcttcatcaagcggcagttggtggagacaagacagataacgaagcacgtggctcagatcctggactctcgcatgaacacgaagtacgacgagaacgacaaattgatccgcgaggtcaaggttattacgctcaagagcaaacttgtcagcgatttccgcaaggacttccagttctacaaggtgagggagattaacaactaccaccatgcacatgatgcctacttgaacgcagtggtggggaccgcgcttattaaaaagtaccctaagttggagtcagagttcgtttatggggactacaaggtgtacgacgtccggaagatgattgcaaagtctgaacaggaaatcgggaaggccaccgccaaatatttcttctacagtaacattatgaatttttttaagactgaaattactctcgcaaacggcgagatcaggaagcgtcccctcatcgagacaaacggggagaccggggagatagtctgggacaaggggcgggacttcgctacggtgaggaaggtgctctcgatgccacaagtgaacatcgtcaaaaagacagaggtgcagaccggtggcttctcaaaggagtcaatcctgccaaaacgtaacagcgacaagctcatcgcccgcaagaaagactgggaccctaagaagtatggtgggttcgactcaccgacggtcgcatactccgttctggtcgtggcaaaggtggaaaagggcaagtccaaaaaactgaaatccgtgaaggagttgcttggcattaccatcatggaacgcagcagcttcgagaagaaccccattgacttcctggaggctaaagggtacaaggaggtcaagaaagatttaattattaagctacctaagtacagcttgttcgagctggagaacggccgaaaacgaatgctcgcatccgccggggaacttcaaaagggcaacgagcttgcgctgccctccaagtacgtgaacttcctgtacttggcatcccactacgagaaactcaagggtagcccagaggacaacgagcagaagcagctattcgtggagcagcacaagcactacctcgacgagataatcgagcagatcagtgagttcagtaagcgggtgatactcgcggacgccaacttggacaaggtgcttagtgcctacaacaagcaccgtgacaagcccatccgagaacaggctgagaacatcatccaccttttcactctgacaaacctcggtgctcccgccgccttcaaatacttcgacactaccatcgacaggaagcgctacacatctacgaaggaagttcttgacgctacgcttattcatcagtctatcacagggctgtacgagacaaggatcgaccttagccaactcggcggggat

SEQ ID NO: 72 - DNA, 4519 bp, Rubus occidentalis
  source 1..4519: mol_type = genomic DNA
gttatctactaacatgccttatcatttgcactaaatgaatatatgatcgaccacattctttcctaaatccaaattactaagcattatcaatttgctatttagttgtctttgttttctccatgatgatgaggtagtaacattgtcatgtgaatcgaagtttagtgaaatcaaatgatttgattcgtttgtaatcaattaaacaatagttctttcatgtggttataaacaatagttccataattgtaatctcaattaaagttcatctaaattatacctcctttccaattaagtttacattgctaagtttttttttttataatatatatatatatatatatatattacagtgcatgattctatttgattgataaaaaatatatttataaagataaaattataattgtaaggtaattcattgataagatatttatatgtaaagctattaattataattgtaagtataattaattgataagttttaaagttgagtaagcaaagtcattgaatctaagatgagtttaatagactcttgcctaaaatgagtaatttcttaatgatgtgtatatgatgtggcatgctatatatgattgatacctccacatcttaggtcttagagcatcttcaacagttttcctaaaaaatctttattatagggactcaaaagttaaaatttctatattttttctttcgtagttccaacagtttttctatcatattctctataatttattatctacaattaaatatggtattatttttaaatttaaaaagtataatcaaataattaatgatgaaaatgaatatatttggaaagaggaaagagaagagggtaaaattttccaaaaagatgtttctctataattttagagtttgctataaatatagagatttttgttttctctctcatcactatctctaaaatagagataggtatagggaagctattggagtaaaaaaatacacaatttttcctaaactagagaaaaaataaattatagggaagctgttggagatgctcttaatgaagttaacaatctttttccattcagcatacatgtcataccttatagcctcatttcacatatatttcacccaaaacttacctccttaattacctctttgggtagtgctagaaggcctcaattaaggcctaacttttgaggtctcaattaagacgtaacttttgagacctcaatcacatgcatgtggcggttcatgtcatattcatgtcatcattttctatcgcatcatattttgcaataactatttataatttttttttaattagattgtactcatatatttatcttaaaatacacaaaaaaaaaattcctagaaaactggtgtatttatgtcaatatatgcaaatgctatatcatttttggtgtttttagataattgttaaagtgttgtttcaaaaatggtgtaccaaacgtatttttgctactagaaaactatgtcatttttgaaaacaaagaactgtatttgtgaagatatcaagcgcaaatttttttttttcctttttgaaaaccaaccacgtactaagagtcatgcaagagtcaacctgatcaatggttcgagttgataattttataacattctgtccataaagaagagaacattttcatctagaaaaagcattgaaccccttgcatcgatgtgacatgcacagagagaaagaaaggatgggaggtgggggactaacaatgttttaatttctaatgtccatgatattctattcatttatactcttcttcttattgctctctttcaagtttcaacacacttagagatctaaaccaagtactggttagcgaacttgaaattactataaatatagagcaatacagggaaaggcaattataagaagtaccaattcttcctgagttttgagttttcttatccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatgacctcggtcttcctgtcgctgccgtttacttcaatgcgcagagagaaacagcagcgagaagacgctagctagctagcagcaggaggcaggccagacccccaaaaattccaggctataaccataatatctaattaaataagagaaaagggtggttaatgatttaatcctccctattgttaggttattaggctaaaagtacttaatttcccaaagagagctgtgagtgtttgtgtctctcatttgatttgtgctctctccatcatcgaatggttataagtttgttctgatttctgatcagttcaatgtatctgtaaagtatctatattcgtatattctatggggttcttgtttttggttttgaatctttgatgatcaactagaactgtatagggacacgtggaacaaacccattatctctctttcgcactcttcttctagtttaagtttagggttttggtttccatttcatttcaaccggaaaatcagaaggaatcaacactatgcaccaatcaagtctgcatgcgggccccacactgtcatgtccgatcttaaataactaattaaccgttggtattgtatgttcaaaagtaagtggaaaaatcactgcttaattattaattaaaaatgttatctatgcactttagtagaacgaaaaaataaattaaaaatgaaacacaacatcattttgacatgacacaagcgtgatatttacattacaattaaaataataaattcatattatgtcgatgcctttttacatttctcgttcacaaattatttttttttttgaggaaaatataaactttattaattaatataaaaggattacaatccttagcatgagcatcttaagaaagagcattatagaccacacaaatgacctaatcgatgagtctaaacctagaactctaatcgatgattcaaaacctagaacaatacatacaccgtgcattactgctatcatacaaagtgtttatgtgaacggcattgactatatgttatctacgggatgattacgattttcatttctaaatgccttaatagttttgtttgacaaactagaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacattttcttttaaaattcgttaacatgtagaaagaaataccactaaattgatgcttagttagtatacaatttctagttccacttagttagattatagttattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggaagggaagagagagctgggttcgtgaggtggctatcggagatggaggtgcagcggtgataagctaggctcaggaggagtagcggagagcagtgatgggattgcaggtgaggctcgagcacgggctcgaggacgacgc

SEQ ID NO: 73 - DNA, 519 bp, Rubus occidentalis
  source 1..519: mol_type = other DNA
atggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatgacctcggtcttcctgtcgctgccgtttacttcaatgcgcagagagaaacagcagcgagaagacgctag

SEQ ID NO: 74 - protein, 172 aa, Rubus occidentalis
MARMSEPLVVGRVIGDVLDSFTPTTKMIVTYTTRHVSNGYELLPSAVTTKPRVEIQGGDMRSFFTLVMTDPDAPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 75 - DNA, 549 bp, Rubus occidentalis
  source 1..549: mol_type = genomic DNA
tccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatg

SEQ ID NO: 76 - DNA, 220 bp, Rubus occidentalis
  source 1..220: mol_type = genomic DNA
tccatctttgattcacctttacgactccctctctctctctctctctctctctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatga

SEQ ID NO: 77 - DNA, 140 bp, Rubus occidentalis
  source 1..140: mol_type = genomic DNA
ctctctctctctctctctctctctctctctctctctctctctcttgtaagtcccaataatggcaagaatgtcggaacccttggttgttggaagggtaattggagatgttcttgattccttcaccccaactaccaaaatga

SEQ ID NO: 78 - DNA, 100 bp, Rubus occidentalis
  source 1..100: mol_type = genomic DNA
ctctctctctctctctctctctcttgtaagtcccaataatggcaagaatgtcggaacccttggttgttggaagggtaattggagatgttcttgattcctt

SEQ ID NO: 79 - DNA, 60 bp, Rubus occidentalis
  source 1..60: mol_type = genomic DNA
ctcttgtaagtcccaataatggcaagaatgtcggaacccttggttgttggaagggtaatt

SEQ ID NO: 80 - DNA, 223 bp, Rubus occidentalis
  source 1..223: mol_type = genomic DNA
tctctctctctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggt

SEQ ID NO: 81 - DNA, 143 bp, Rubus occidentalis
  source 1..143: mol_type = genomic DNA
gtcccaataatggcaagaatgtcggaacccttggttgttggaagggtaattggagatgttcttgattccttcaccccaactaccaaaatgattgtcacttacactacgaggcacgtctctaatggatatgagctcttaccttc

SEQ ID NO: 82 - DNA, 103 bp, Rubus occidentalis
  source 1..103: mol_type = genomic DNA
gtcggaacccttggttgttggaagggtaattggagatgttcttgattccttcaccccaactaccaaaatgattgtcacttacactacgaggcacgtctctaat

SEQ ID NO: 83 - DNA, 63 bp, Rubus occidentalis
  source 1..63: mol_type = genomic DNA
gaagggtaattggagatgttcttgattccttcaccccaactaccaaaatgattgtcacttaca

SEQ ID NO: 84 - DNA, 223 bp, Rubus occidentalis
  source 1..223: mol_type = genomic DNA
ctctctctctctctctctctctctcttgtaagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgaga

SEQ ID NO: 85 - DNA, 143 bp, Rubus occidentalis
  source 1..143: mol_type = genomic DNA
atggcaagaatgtcggaacccttggttgttggaagggtaattggagatgttcttgattccttcaccccaactaccaaaatgattgtcacttacactacgaggcacgtctctaatggatatgagctcttaccttctgcagtcac

SEQ ID NO: 86 - DNA, 103 bp, Rubus occidentalis
  source 1..103: mol_type = genomic DNA
cttggttgttggaagggtaattggagatgttcttgattccttcaccccaactaccaaaatgattgtcacttacactacgaggcacgtctctaatggatatgag

SEQ ID NO: 87 - DNA, 63 bp, Rubus occidentalis
  source 1..63: mol_type = genomic DNA
ttggagatgttcttgattccttcaccccaactaccaaaatgattgtcacttacactacgaggc

SEQ ID NO: 88 - DNA, 224 bp, Rubus occidentalis
  source 1..224: mol_type = genomic DNA
aagtcccaataatggcaagaatgtcggaacccttggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagaccca

SEQ ID NO: 89 - DNA, 143 bp, Rubus occidentalis
  source 1..143: mol_type = genomic DNA
ggaagggtaattggagatgttcttgattccttcaccccaactaccaaaatgattgtcacttacactacgaggcacgtctctaatggatatgagctcttaccttctgcagtcaccaccaaacctagagttgagattcaaggcgg

SEQ ID NO: 90 - DNA, 103 bp, Rubus occidentalis
  source 1..103: mol_type = genomic DNA
tcttgattccttcaccccaactaccaaaatgattgtcacttacactacgaggcacgtctctaatggatatgagctcttaccttctgcagtcaccaccaaacct

SEQ ID NO: 91 - DNA, 63 bp, Rubus occidentalis
  source 1..63: mol_type = genomic DNA
ctaccaaaatgattgtcacttacactacgaggcacgtctctaatggatatgagctcttacctt

SEQ ID NO: 92 - DNA, 223 bp, Rubus occidentalis
  source 1..223: mol_type = genomic DNA
ggttgttggaagggtaattggagatgttcttgattccttcaccccaactaccaaaatgattgtcacttacactacgaggcacgtctctaatggatatgagctcttaccttctgcagtcaccaccaaacctagagttgagattcaaggcggtgacatgagatcattcttcactctggtgatgacagacccagatgctcctggccctagtgatccttatttgaag

SEQ ID NO: 93 - DNA, 143 bp, Rubus occidentalis
  source 1..143: mol_type = genomic DNA
accccaactaccaaaatgattgtcacttacactacgaggcacgtctctaatggatatgagctcttaccttctgcagtcaccaccaaacctagagttgagattcaaggcggtgacatgagatcattcttcactctggtgatgac

SEQ ID NO: 94 - DNA, 96 bp, Rubus occidentalis
  source 1..96: mol_type = genomic DNA
tgtcacttacactacgaggcacgtctctaatggatatgagctcttaccttctgcagtcaccaccaaacctagagttgagattcaaggcggtgacat

SEQ ID NO: 95 - DNA, 56 bp, Rubus occidentalis
  source 1..56: mol_type = genomic DNA
acgtctctaatggatatgagctcttaccttctgcagtcaccaccaaacctagagtt

SEQ ID NO: 96 - DNA, 223 bp, Rubus occidentalis
  source 1..223: mol_type = genomic DNA
tgagctcttaccttctgcagtcaccaccaaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatc

SEQ ID NO: 97 - DNA, 143 bp, Rubus occidentalis
  source 1..143: mol_type = genomic DNA
gagattcaaggcggtgacatgagatcattcttcactctggtgatgacagacccagatgctcctggccctagtgatccttatttgaaggagcacctgcactgggttgtgacagacattccaggcaccacagatgctacatttgg

SEQ ID NO: 98 - DNA, 103 bp, Rubus occidentalis
  source 1..103: mol_type = genomic DNA
gagatcattcttcactctggtgatgacagacccagatgctcctggccctagtgatccttatttgaaggagcacctgcactgggttgtgacagacattccaggc

SEQ ID NO: 99 - DNA, 63 bp, Rubus occidentalis
  source 1..63: mol_type = genomic DNA
tgatgacagacccagatgctcctggccctagtgatccttatttgaaggagcacctgcactggg

SEQ ID NO: 100 - DNA, 220 bp, Rubus occidentalis
  source 1..220: mol_type = genomic DNA
gatccttatttgaaggagcacctgcactgggttgtgacagacattccaggcaccacagatgctacatttggaagagaggtggtgagctatgagatgccaaggcccaacatcggcatccacaggtttgtgtttgttctcttcaggcagaaacgaaggcagtcagtgaaccctccttcctcaagggatcacttcaacacccgaagcttcgcagcagaaaatg

SEQ ID NO: 101 - DNA, 140 bp, Rubus occidentalis
  source 1..140: mol_type = genomic DNA
acattccaggcaccacagatgctacatttggaagagaggtggtgagctatgagatgccaaggcccaacatcggcatccacaggtttgtgtttgttctcttcaggcagaaacgaaggcagtcagtgaaccctccttcctca

SEQ ID NO: 102 - DNA, 100 bp, Rubus occidentalis
  source 1..100: mol_type = genomic DNA
gctacatttggaagagaggtggtgagctatgagatgccaaggcccaacatcggcatccacaggtttgtgtttgttctcttcaggcagaaacgaaggcagt

SEQ ID NO: 103 - DNA, 60 bp, Rubus occidentalis
  source 1..60: mol_type = genomic DNA
ggtgagctatgagatgccaaggcccaacatcggcatccacaggtttgtgtttgttctctt

SEQ ID NO: 104 - DNA, 204 bp, Rubus occidentalis
  source 1..204: mol_type = genomic DNA
aaacctagagttgagattcaaggcggtgacatgagatcattcttcactctggtgatgacagacccagatgctcctggccctagtgatccttatttgaaggagcacctgcactgggttgtgacagacattccaggcaccacagatgctacatttggaagagaggtggtgagctatgagatgccaaggcccaacatcggcatccac

SEQ ID NO: 105 - DNA, 132 bp, Rubus occidentalis
  source 1..132: mol_type = genomic DNA
cctggccctagtgatccttatttgaaggagcacctgcactgggttgtgacagacattccaggcaccacagatgctacatttggaagagaggtggtgagctatgagatgccaaggcccaacatcggcatccac

SEQ ID NO: 106 - DNA, 724 bp, Pyrus pyrifolia
  source 1..724: mol_type = genomic DNA
gaaaaatataaggagtgctattagctcctcctgaattgacttatccattaatcttttctttttacactctctcttctctctctctctctctctctctctctctctcttaaaatgaaaaaagcctcggagcctccggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctagctagtagctgtacccagaactcctccatccattatccatatatatgttaaataaaggcttctttatagataggctattgtaactcttgtttccc

SEQ ID NO: 107 - DNA, 519 bp, Pyrus pyrifolia
  source 1..519: mol_type = other DNA
atgaaaaaagcctcggagcctccggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctag

SEQ ID NO: 108 - protein, 172 aa, Pyrus pyrifolia
MKKASEPPVVGRVIGDVLDSFTATTKMSVTYNTKLVCNGLELFPSVVTAKPRVEIQGGDMRSFFTLVMTDPDFPGPSDPYLREHLHWIVTDIPGTTDATFGREVVSYEMPKPNIGIHRFVFVLFKQNRRQSINTPSSRDHFSTRSFAAENDLGLPVAAVYFNAQRETAARRR

SEQ ID NO: 109 - DNA, 401 bp, Pyrus pyrifolia
  source 1..401: mol_type = genomic DNA
cttcactgcaacaacaaaaatgtctgtcacttacaacaccaagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcacttcagcactcgaagcttcgcggctgaaaatgacctgggtc

SEQ ID NO: 110 - DNA, 321 bp, Pyrus pyrifolia
  source 1..321: mol_type = genomic DNA
aagctagtctgcaatggacttgagctctttccttctgttgtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaatcgaagacaatcaatcaacacaccttcctcgagggatcac

SEQ ID NO: 111 - DNA, 243 bp, Pyrus pyrifolia
  source 1..243: mol_type = genomic DNA
gtcacagccaaacctagagttgagattcaaggaggggatatgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatattggcatccacaggtttgtgtttgttctcttcaagcagaat

SEQ ID NO: 112 - DNA, 165 bp, Pyrus pyrifolia
  source 1..165: mol_type = genomic DNA
atgagatctttctttactttggtgatgacagacccagattttcctggccctagtgatccttatctaagggagcacctgcactggattgtgacagacattccaggcaccacagatgccacatttggaagagaggtggtgagttatgagatgccgaagcccaatatt

SEQ ID NO: 113 - DNA, 134 bp, Pyrus pyrifolia
  source 1..134: mol_type = genomic DNA
cctggccctagtgatccttatctaagggagcacctgcactggattgtgacagacattccaggcaccacagatgccacatttggaagagaggtggtgagttatgagatgccgaagcccaatattggcatccacag

SEQ ID NO: 114 - DNA, 887 bp, Prunus serotina
  source 1..887: mol_type = genomic DNA
aagcaatataagaagtacatctcaaattcctcctgagttcttatccattaatctttgatcaatagctacaccttctttctcactcttcctcattctctcctagtcacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagccgaaaacgacctgggtcttcctgtcgctgccgtttacttcaattgccagagagaaacagcagctagaagacgctagctagctacccaaaaccccaaaggtcctccataatatgttaattttaaataaagttattatcaagtgtgtttcatcatcctcccctatcgtcagagtagtattaggccaaaactactcacgtgtaacaagaataatttccacgagagagcagtgtgtgtctggctctgtcattttactgctctccaatcggactttatgaactaatttgcagtaagtttgaagtctcatctgagtatctctaaaaaaaaaaaaaa

SEQ ID NO: 115 - DNA, 519 bp, Prunus serotina
  source 1..519: mol_type = other DNA
atggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagccgaaaacgacctgggtcttcctgtcgctgccgtttacttcaattgccagagagaaacagcagctagaagacgctag

SEQ ID NO: 116 - protein, 172 aa, Prunus serotina
MARMSEPLVVGRVIGDVLDCFTPTTKMSVTYNTKLVCNGYELYPSAVTNKPRVEIQGGDMRTFFTLVMTDPDVPGPSDPYLREHLHWIVTDIPGTTDATFGREVVSYEMPRPNIGIHRFVFVLFKQKRRQSVNPPSSRDHFSARSFAAENDLGLPVAAVYFNCQRETAARRR

SEQ ID NO: 117 - DNA, 224 bp, Prunus serotina
  source 1..224: mol_type = genomic DNA
agctacaccttctttctcactcttcctcattctctcctagtcacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttctgctgtcaccaacaaacctagagttgagattcaaggagg

SEQ ID NO: 118 - DNA, 144 bp, Prunus serotina
  source 1..144: mol_type = genomic DNA
cacaaaaaaaatggcaagaatgtctgagcctctggttgttgggagagtgataggagatgttcttgattgtttcaccccaacaacaaaaatgtctgtcacttacaacaccaagctagtctgcaatggatatgagctctatccttc

SEQ ID NO: 119 - DNA, 104 bp, Prunus serotina
  source 1..104: mol_type = genomic DNA
tgtctgagcctctggttgttgggagagtgataggagatgttcttgattgtttcaccccaacaacaaaaatgtctgtcacttacaacaccaagctagtctgcaat

SEQ ID NO: 120 - DNA, 63 bp, Prunus serotina
  source 1..63: mol_type = genomic DNA
gggagagtgataggagatgttcttgattgtttcaccccaacaacaaaaatgtctgtcacttac

SEQ ID NO: 121 - DNA, 223 bp, Prunus serotina
  source 1..223: mol_type = genomic DNA
tgagctctatccttctgctgtcaccaacaaacctagagttgagattcaaggaggagatatgagaactttctttactctggtcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 122 - DNA, 143 bp, Prunus serotina
  source 1..143: mol_type = genomic DNA
gagattcaaggaggagatatgagaactttctttactctggtcatgacagacccagatgttcctggccctagtgatccttatttaagggagcacctgcactggattgtgacagatattccaggcaccacagatgccacatttgg

SEQ ID NO: 123 - DNA, 103 bp, Prunus serotina
  source 1..103: mol_type = genomic DNA
gagaactttctttactctggtcatgacagacccagatgttcctggccctagtgatccttatttaagggagcacctgcactggattgtgacagatattccaggc

SEQ ID NO: 124 - DNA, 63 bp, Prunus serotina
  source 1..63: mol_type = genomic DNA
tcatgacagacccagatgttcctggccctagtgatccttatttaagggagcacctgcactgga

SEQ ID NO: 125 - DNA, 134 bp, Prunus serotina
  source 1..134: mol_type = genomic DNA
cctggccctagtgatccttatttaagggagcacctgcactggattgtgacagatattccaggcaccacagatgccacatttggaagagaggtggtgagctatgagatgccaaggcccaacattggcatccacag

SEQ ID NO: 126 - DNA, 4519 bp, Prunus persica
  source 1..4519: mol_type = genomic DNA
ctttatcttactgataagatctaaagaaaaccagtaggtggataagatcaaatcactttgccatttactcttcggtaaaatatagcaataattgactggaaatgtgacgcagacagattgcacgtacagattacacagattgcagttccataacaggtgtggttcatgagaatatataataaaaataatatatacaaaaaggtaaacttttaagctcaactcaactgtatcaaccttacataatatttataaaaaaatggaaacgatagaggaaggaagcatcgatcctgagggaaaaaagaggaaattaacaattttataaataaaaaaggaaaaaaaaaaaaaggagcaaagaaagcaaaagaatggaaaattcaaacagggggggtgcatgcagtatagtatagaacactttcacatgaggtccagcttggaatatacagaagaaaaaaaaagtagtttggcttcttgtaatggaaaattcatcttctttctcattgccattcctggaacttctctgaaaatcacttttattttaatctgagttatatatattgagatgacctctcacagcaaaagcagtatagcagcaggccaggcactcatagaggtggagttcctttatgccctcagcctgtcttcaaagatagagaatctctctgaaaatgttgaaattccctctcgtgttccatatttatatatgcacacattcaacattttcttctgattacccaaactgcaaaaggtgaatttcctttataagcttgtgaatgttcagcaagaatcctagaaatttagctgtttttgctgtcttgctttttatatatatatatacatatataatagcatgaattgacagccatggaactgtaaggaccccaccatggcatgcaggattgtatataataatggttttctggcctgaccatggaaaaacatccccccgttttgaataaaaatgttcaataattaagtgcacagccaatcaaatcaagaaccaattccaagggacccaattaatatgagaggcacagatatataacataagtatattatgcatattgtcctaaataagcaaaaattattgcatacaccacatttttctcagttaatcatagaaagagtgttaattagtcgtcataagagcacataattcatgtgttagactcaacatgtcacccaacttattacgtttgattttgatcaggtaaatcattacaagaaaaattggctaatgtgatgaaattaaaagtaaagtttctaaattcgtcacatgtagttagaatatgtgaagaatttatttggtcgtcacttataaaatgatcaagagaccaacagtttttgggaaagaccaaagtcgtcacaaacatgagcgcagttctaggtgggaaagactaacaagtatttgtgatgccaacgggtcacatataaaactctaatgcgtcacaaatattaaatgttgaaatctatagtgaaaaaagagaatacaaaaagaattagaggaaaaatttccctacccaaaagaaaatatatatttcaggaattcttttggtcatcgcatataatgaactatgacaaattatgtgaggatcgggcctcacttattcacatctaatattgaagaaaccatgttatatatagaattaatacaactgttatatatacaggaatttttctggaaacgataccactaaactgtgttgatgtgacatagaaaaaagagggtgggaggtgggggactggctaacaatgttttctagagatgtccgtattctatgcctttatactctttcttcttcttaatgcccgtctcaaaacgcataatagagatctaaagtaactcgcttgaaaacactataaatatacaaccttacaggaattccactaccaagcaatataagaagtacatctctaattcctcctgagttcttatccattaatctttgatcaatagctacactttctttctcactcctcctcattctctctgtctccaaaaaatggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacattggcatccacaggtttgtgtttgttctcttcaagcagacaagaaggcagtctgtgaaccctccttcctcaagggatcatttcagtgctcgaagctttgcagctgaaaacgacctgggtcctcctgtcgctgccgtttacttcaattgccagagagaaacggcagctagaagacgctagctagctacccaaaaccccaaagctcctccataatatgttaattttaaataaaattattatcaagtgtgtttcatcatcctcccctgtcgttagagtagtattaggctaaaactactcacatgtaaccagaataatttccagtcaagatagagctaggtgcgtttggctctgtcattttgctgctctctaatcggacttaatgaattaatttgcagaaagtttgaagtctcacctgactatctgtgaaatatctctctatggtgtttttaatttatattttattataaggggacaggagcaaccaaactttgtcctttttaagttagggttttggcttccatttcgactggaaaagtagaagaccctctattattgacttcatctcttcttcccagcttagggttttgggttgcacgtttcaagcacaaaaatatgtgcgatttgacagcagtcagtcacaccacatccagctatgtcgacgcttgtaatgaagaggcaactagttttatttttgcttttgagaacgaagaagcaagtattatagatgcatggtaaagaaaaatacttgattccgtcaaagaaagtagaaattcatactattttattgctaattatattttgatatgtgtgtagagatttctctttttgtatttttgataaattatttttgcatatatgtcgaattgtgatttgcagaaagtaggaatatctcattccatggtaaatttataatttataatatgaattatttagaatatgaaaaataatttactgcaaaagtttcacttggaaacccaattttcatgcaccctagtttttttttttttttttacacaaaccgaacattaaaaaactaaaccctaaaaactttgtcgattaagaaccaaaactttgtcatttgataccaaaaaatccacacattttatgccctaatactatatgagttattaaattattggcctttttatccatgaatttatgggttttatttatttcccctcttcattttgcatatgggtttaaacttttcccaataatggagccttgactgttttgagttgtcaacaaaaagcgataatcaacgggatcttgtgcagtggaatgacattaaattgcaggcaaggggtcacaaattcaaaccccatggcacttagttgtgtgtgtgagaaactctatttcttgtagtttagactattacttgtacttaaagaaaaataaaaattaaacaaagatatacgttatctcatctgaatcgatgcacaagttgcacatgactaaaacaacaatatatatgtccaagtgtttatctataggggcggagctagaattgtaaagttaaggggtcgaaaaagaaaatacaagtatataaaacacttaagtattcaataattatatgtttagagctaaatatttctaattgaatcatgtctaatttgttttagaggaaattttaatttaaattataagttgtttattacaaattctatatattatatataatataatagaagatggataaaaatctaaggaggccagggccactccagaactagaagtggctctgccactgcttatctaattaattaaggatccatatattcaagtccctaaatgcatacacgtattagatttaattacaaccgtatgaaattactctaccaaatcatatggttagatttccctcttgcagttgagagtcggattagccatcaatggacaatctaacataatgatgagacggaattatacaaaaaatatatatgaatgtctagcggatctttaatttgcagtgataatttcaggtattatacttatgtttctccattgcagctgtctacgttcttgagattcaagatgtgtattatacttgacgtattggtagacagtagacataattgtatttcatgaggtacgcatgcaatattatgatgattaacataattatatatattattaatcaatttgaattactttttaagatttcatattggcaggtagct

SEQ ID NO: 127 - DNA, 519 bp, Prunus persica
  source 1..519: mol_type = genomic DNA
atggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacattggcatccacaggtttgtgtttgttctcttcaagcagacaagaaggcagtctgtgaaccctccttcctcaagggatcatttcagtgctcgaagctttgcagctgaaaacgacctgggtcctcctgtcgctgccgtttacttcaattgccagagagaaacggcagctagaagacgctag

SEQ ID NO: 128 - protein, 172 aa, Prunus persica
MARISEPLVVGRVIGDVLDCFTPTTKMSVTYNTRLVCNGYELYPSAVTTKPRVEIQGGDMRTFFTLIMTDPDVPGPSDPYLREHLHWIVTDIPGTTDATFGREVVSYEMPRPNIGIHRFVFVLFKQTRRQSVNPPSSRDHFSARSFAAENDLGPPVAAVYFNCQRETAARRR

SEQ ID NO: 129 - DNA, 223 bp, Prunus persica
  source 1..223: mol_type = genomic DNA
atagctacactttctttctcactcctcctcattctctctgtctccaaaaaatggcaagaatatctgagcctctggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggagg

SEQ ID NO: 130 - DNA, 143 bp, Prunus persica
  source 1..143: mol_type = genomic DNA
tctccaaaaaatggcaagaatatctgagcctctggttgttgggagagtgataggagatgttcttgattgtttcaccccaacaacaaaaatgtctgtcacttacaacaccaggctagtctgcaatggatatgagctctatcctt

SEQ ID NO: 131 - DNA, 103 bp, Prunus persica
  source 1..103: mol_type = genomic DNA
tatctgagcctctggttgttgggagagtgataggagatgttcttgattgtttcaccccaacaacaaaaatgtctgtcacttacaacaccaggctagtctgcaa

SEQ ID NO: 132 - DNA, 223 bp, Prunus persica
  source 1..223: mol_type = genomic DNA
ggttgttgggagagtgataggagatgttcttgattgtttcaccccaacaacaaaaatgtctgtcacttacaacaccaggctagtctgcaatggatatgagctctatccttctgctgtcaccaccaaacctagagttgagattcaaggaggagatatgagaactttctttactctgatcatgacagacccagatgttcctggccctagtgatccttatttaagg

SEQ ID NO: 133 - DNA, 143 bp, Prunus persica
  source 1..143: mol_type = genomic DNA
accccaacaacaaaaatgtctgtcacttacaacaccaggctagtctgcaatggatatgagctctatccttctgctgtcaccaccaaacctagagttgagattcaaggaggagatatgagaactttctttactctgatcatgac

SEQ ID NO: 134 - DNA, 104 bp, Prunus persica
  source 1..104: mol_type = genomic DNA
ctgtcacttacaacaccaggctagtctgcaatggatatgagctctatccttctgctgtcaccaccaaacctagagttgagattcaaggaggagatatgagaact

SEQ ID NO: 135 - DNA, 63 bp, Prunus persica
  source 1..63: mol_type = genomic DNA
tagtctgcaatggatatgagctctatccttctgctgtcaccaccaaacctagagttgagattc

SEQ ID NO: 136 - DNA, 223 bp, Prunus persica
  source 1..223: mol_type = genomic DNA
tgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccgggcaccacagatgccacatttggaagagaggtggtgagctatgagatgccaaggcccaacatt

SEQ ID NO: 137 - DNA, 143 bp, Prunus persica
  source 1..143: mol_type = genomic DNA
gagattcaaggaggagatatgagaactttctttactctgatcatgacagacccagatgttcctggccctagtgatccttatttaagggagcacctgcactggattgtgacagatattccgggcaccacagatgccacatttgg

SEQ ID NO: 138 - DNA, 103 bp, Prunus persica
  source 1..103: mol_type = genomic DNA
gagaactttctttactctgatcatgacagacccagatgttcctggccctagtgatccttatttaagggagcacctgcactggattgtgacagatattccgggc

SEQ ID NO: 139 - DNA, 132 bp, Prunus persica
  source 1..132: mol_type = genomic DNA
cctggccctagtgatccttatttaagggagcacctgcactggattgtgacagatattccgggcaccacagatgccacatttggaagagaggtggtgagctatgagatgccaaggcccaacattggcatccac

SEQ ID NO: 140 - DNA, 4519 bp, Prunus avium
  source 1..4519: mol_type = genomic DNA
tggtgcatgcagtatggcatagaacactttcacatgaggtccagcttggaatatacagaaaaaaagtagtttggcttcttgtaatggaaaattcaccttctttctcattgccattcctggaacttctctgaaaatcacttttattttaatctgagttatatatatattgagatgacctctcacagcaaaagctgttgcagcaggccaggcactcattgaggtggagttcctttatgccctcagcctgtcttcaaagatagagaatctttctgaaaatgttgaaattctctcgtgtaccatatttatatatgcacacattcaacattttcttctgcttaccctaactgcaaaaggtgaatttcctttataagcttgtgaatgttcagcaagaatcctagacattttgctatttttgctgtgtattgctttttatatatatatatatacatatataatagcatgaattgacagccatggaaatgtaaggaccccaccatgcaggattgtatataataatggttttctggcctgaccatggaaaaatatccccccgttttgaataaaaatgttcaataattaagtgcacagccaatcaaatcaagaaccaattccaagggacccaattaatatgagaggcacagatatataacatgagtatattatgcatattatcctaaataagcaaaaattattgcgtgcaccacatttttcttagttaatcatagagagattgttaattagtagtataagggctcgttcggcactgcttatctaggaagcacttctacttataattgtttctaccaaaagcgcttttattataaatacgttgaaattttcataagaaattaaagtgcttcttggaaaagcacttgggagtgcttcctgaagatgcacatggtatgtgcttctccagaaagcgcttctatgacccaaaatcgcttctaaacttttctggcaaacattgttagaagcgcttttggttgtcagaaagcgcttttaactgttccaaaagcactcccaaacgagtcctaaatctcaagtcgtgataagatcctaaatttttttcacttgttgctaattaattttgagttggatgttctaactatactatgatattagagcacacaattcatgtgttagactcaacacgtcacccaacttattacgtttgattttgatcagttagatcattacaagaaaatttttgactaatgtcatgaaattaaaattgaagttggtaaattcgtcacatgtgattggaatatgtgaggaatttattttgtcgtcacttataacatggccaagaaaacagcagtttgtgggaaagaccaaagtcctcacaaacatgagcgtgattctaggtgggaaagactaacaattatttgtggtgccaatggggtcacatataaaattttaatgcgtcacaaatattaaatgtgggaatctctagtgaaaaaagaaaatacaaaaagaatgagaggaaaaatttccctacccaaaagaaaatatatatatttctggaattcttttggtcatcacatataattaattatgacaaattatgtgaggatcgggcctcacttattcacatctaatatttaagaaaccatgttatagaaagaattaacatataaaatacattaatacaaccgttatatatataggaatttttctggaaacgataccactaaactctattgatgtgacatagaaaacagagggtgggaggtgggggactggctaacaatgttttctagagatgtccatattctatgcctttatactctttcttcttcttaatgcctgtctcaaaacacataatagagatctaaagtaactcgcttgaaaacactataaatacaaccttacatgaattccatttccaagcaatataagaagtacatctctaattcctcctgagttcttatccattaatctttggtcaatagctacaccttctttttcactcctcctcattctctctttctctcagaaaaatggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagctgaaaacgacctgggtcttcctgtcgctgccgtctacttcaattgccagagagaaacggcagctagaagacgctagctagctacccagaaccccaaagctcctccataatatgtgaattttaaataaaattattatcaagtgtgtttcatcatcctcccctgtcgttagagtagtattaggctaaaactactcacatgtaaccagaataatttccaatcaagagagagcaatgtgcgtttggctctgtcattttgctgctctccaatcggactttatgaattaatttgcagaaagtttgaagtctcacctgactatctgtgaaatatctctcgatggggtttttaatttatattttattataaggggacaggaggaaccaaacgtccctctttttgtcctttttaagttagggttttggcttccatttcgactggaaaagtagaagagcctctattattgacttcatagttcatctcttcttcccagtttagggttttgggttgcacgtttcaagctgaaaaatatgttcgatttgacagcagtcagtcacaccacatccagctatgtcgactcttgtaatgaagaggcaactagttttttttttgcttttgagaacgaagaagcaagtattatagatgcatggtaaagaaaaatacttgactccgtcaaagaaagtacgaattcctactacttgattgcattagagatttctcttttcgtgtttttgataaattatttttgtatatatgtagaactgtgatttacagaaagtaggaatgcctccttccatggtaaatttatgattaacaatatgatttagaataggaaaaacaatttattgtaaaagtttcacttagaaacccaattttcatgcaccctatttttttttacacaaaccgaacattaagaaactaaaccctaaaaactttgtcaattaagaaccaaaactttgtcatttgataccaaaaaagccacacattttatgcttaagaaccctaatactatatgagttattaaattattggccttttcatccatgaatttatgggttttattcttcattttgcatatgggtttaaacttttcgcaataatggagtctcgactattttgagttgtaaacaaaaagcaatagtcaacggaatctagcgcagtggaaaatgatattaaattgcagacaagaggtcacaaattcgaacccccatggcacttagttgtgtgtgtgagaaaccccctctcttgtagtttagactatcacttgtactgaaagaaaaaaaaaataaacaaagatatacgttatctcatctaaatcgatgcacaagttgcacatgattaaaacaacaatatatatgtccaagtgcttattcacaggggcgaagctagaattgtaaagttgaggggtcaaagaagaaaatacaagtatataaaacacctaagtattcaataattacatgtttagagctaaatatttctaattgagtaatgtctaatttgttttagaggaaattttaacttaaattataagttgtttattacaaattctatatgttatatataatatatctggggggccagagctactccaggactagaagtggctccgccactacttatctaattaattaaggatccatatattcaagtctccaaatgcaaacacatactagatttaattgcaaccttatgaattactctaccaaatcatagggttagatttccctcttgcagttgagcatcggattagccatcaatggacaatctaacataatgatgagacggaattatacaaaatatatatatgaatatctagcggatctttaatttgcagtgataatttcaggtattatacttgacgtattggtagacagtattggtagacagtagacataattgtattatacttgacgtattggggtagacagtattggtagacagtagacttatgttttctccattgcagctgtctaagttcttgaaattcaagatgtgtattatacttgacgtattggtagacagtagacataattgtatttcatgaggtacgcatgcaatattatgatgattaacataattata

SEQ ID NO: 141 - DNA, 519 bp, Prunus avium
  source 1..519: mol_type = other DNA
atggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacaggtttgtgtttgttctcttcaagcagaaaagaaggcagtctgtgaaccctccttcctcaagggatcatttcagtgctcgaagcttcgcagctgaaaacgacctgggtcttcctgtcgctgccgtctacttcaattgccagagagaaacggcagctagaagacgctag

SEQ ID NO: 142 - protein, 172 aa, Prunus avium
MARTSEPLVVGRVIGDVLDCFTPTTKMSVTYNTKLVCNGYELYPSAVTTKPRVEIQGGDMRTFFTLIMTDPDVPGPSDPYLREHLHWIVTDIPGTTDATFGREVVSYEMPRPNIGIHRFVFVLFKQKRRQSVNPPSSRDHFSARSFAAENDLGLPVAAVYFNCQRETAARRR

SEQ ID NO: 143 - DNA, 223 bp, Prunus avium
  source 1..223: mol_type = genomic DNA
agctacaccttctttttcactcctcctcattctctctttctctcagaaaaatggcaagaacgtctgagcctctggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggagg

SEQ ID NO: 144 - DNA, 143 bp, Prunus avium
  source 1..143: mol_type = genomic DNA
tctcagaaaaatggcaagaacgtctgagcctctggttgttgggagagtaataggagatgttcttgattgtttcaccccaacaacaaaaatgtctgtcacttacaacaccaagctagtctgcaatggatatgagctctatcctt

SEQ ID NO: 145 - DNA, 103 bp, Prunus avium
  source 1..103: mol_type = genomic DNA
cgtctgagcctctggttgttgggagagtaataggagatgttcttgattgtttcaccccaacaacaaaaatgtctgtcacttacaacaccaagctagtctgcaa

SEQ ID NO: 146 - DNA, 63 bp, Prunus avium
  source 1..63: mol_type = genomic DNA
gggagagtaataggagatgttcttgattgtttcaccccaacaacaaaaatgtctgtcacttac

SEQ ID NO: 147 - DNA, 223 bp, Prunus avium
  source 1..223: mol_type = genomic DNA
ggttgttgggagagtaataggagatgttcttgattgtttcaccccaacaacaaaaatgtctgtcacttacaacaccaagctagtctgcaatggatatgagctctatccttctgctgtcaccaccaaacctagagttgagattcaaggaggagatatgagaactttctttactctgatcatgacagacccagatgttcctggccctagtgatccttatttaagg

SEQ ID NO: 148 - DNA, 143 bp, Prunus avium
  source 1..143: mol_type = genomic DNA
accccaacaacaaaaatgtctgtcacttacaacaccaagctagtctgcaatggatatgagctctatccttctgctgtcaccaccaaacctagagttgagattcaaggaggagatatgagaactttctttactctgatcatgac

SEQ ID NO: 149 - DNA, 103 bp, Prunus avium
  source 1..103: mol_type = genomic DNA
tgtcacttacaacaccaagctagtctgcaatggatatgagctctatccttctgctgtcaccaccaaacctagagttgagattcaaggaggagatatgagaact

SEQ ID NO: 150 - DNA, 223 bp, Prunus avium
  source 1..223: mol_type = genomic DNA
tgagctctatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 151 - DNA, 143 bp, Prunus avium
  source 1..143: mol_type = genomic DNA
gagattcaaggaggagatatgagaactttctttactctgatcatgacagacccagatgttcctggccctagtgatccttatttaagggagcacctgcactggattgtgacagatattccaggcaccacagatgccacatttgg

SEQ ID NO: 152 - DNA, 103 bp, Prunus avium
  source 1..103: mol_type = genomic DNA
gagaactttctttactctgatcatgacagacccagatgttcctggccctagtgatccttatttaagggagcacctgcactggattgtgacagatattccaggc

SEQ ID NO: 153 - DNA, 519 bp, Prunus armeniaca
  source 1..519: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacattggcatccacacgtttgtgtgtgttctcttcaagcagaaaagaaggcagtctgtgaaccctccttcctcaagggatcatttcagtgctcgaagcttcgcagctgaaaacgacctggatcttcctgtcgctgccgtttacttcaattgccagagagaaacggcagctagaagacgctag

SEQ ID NO: 154 - protein, 172 aa, Prunus armeniaca
MARMSEPLVVGRVIGDVLDCFTPTTKMSVTYNTRLVCNGYELYPSAVTTKPRVEIQGGDMRTFFTLIMTDPDVPGPSDPYLREHLHWIVTDIPGTTDATFGREVVSYEMPRPNIGIHTFVCVLFKQKRRQSVNPPSSRDHFSARSFAAENDLDLPVAAVYFNCQRETAARRR

SEQ ID NO: 155 - DNA, 173 bp, Prunus armeniaca
  source 1..173: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatccttctgctgtcaccaccaaacctagagttgagattcaaggagg

SEQ ID NO: 156 - DNA, 133 bp, Prunus armeniaca
  source 1..133: mol_type = other DNA
atggcaagaatgtctgagcctttggttgttgggagagtgataggagatgttcttgattgtttcaccccaacaacaaaaatgtctgtcacttacaacaccaggctagtctgcaatggatatgagctgtatcctt

SEQ ID NO: 157 - DNA, 103 bp, Prunus armeniaca
  source 1..103: mol_type = other DNA
tgtctgagcctttggttgttgggagagtgataggagatgttcttgattgtttcaccccaacaacaaaaatgtctgtcacttacaacaccaggctagtctgcaa

SEQ ID NO: 158 - DNA, 223 bp, Prunus armeniaca
  source 1..223: mol_type = other DNA
tgagctgtatccttctgctgtcaccaccaaacctagagttgagattcaaggaggagatatgagaactttctttactctgatcatgacagacccagatgttcctggccctagtgatccttatttaagggagcacctgcactggattgtgacagatattccaggcaccacagatgccacatttggaagagaggtggtgagctatgagatgccaaggcccaacatt

SEQ ID NO: 159 - DNA, 143 bp, Prunus armeniaca; identical to SEQ ID NO: 151
  source 1..143: mol_type = other DNA

SEQ ID NO: 160 - DNA, 103 bp, Prunus armeniaca; identical to SEQ ID NO: 152
  source 1..103: mol_type = other DNA

SEQ ID NO: 161 - DNA, 132 bp, Prunus armeniaca
  source 1..132: mol_type = other DNA
cctggccctagtgatccttatttaagggagcacctgcactggattgtgacagatattccaggcaccacagatgccacatttggaagagaggtggtgagctatgagatgccaaggcccaacattggcatccac

SEQ ID NO: 162 - DNA, 667 bp, Malus domestica
  source 1..667: mol_type = genomic DNA
ggagtgctattagctcctcctgaattgacttatccattaatcttctctttttacactctctcttctctctctctctctctctctctctctctctctctctctctctctctctctctcttaaaatgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctagctagtagctgtacccagaactcctcc

SEQ ID NO: 163 - DNA, 519 bp, Malus domestica
  source 1..519: mol_type = other DNA
atgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgctgccgtctacttcaacgcgcagagagaaactgcagctagaagacgctag

SEQ ID NO: 164 - protein, 172 aa, Malus domestica
MKRASEPLVVGRVIGDVLDSFTATTKMSVTYNTKLVCNGLELFPSVVTAKPRVEIQGGDMRSFFTLVMTDPDFPGPSDPYLREHLHWIVTDIPGTTDATFGREVVSYEMPKPNIGIHRFVFVLFKQNQRQSINTPSSRDHFSTRSFAAENDLGLPVAAVYFNAQRETAARRR

SEQ ID NO: 165 - DNA, 491 bp, Malus domestica
  source 1..491: mol_type = genomic DNA
ctctctctctctctcttaaaatgaaaagagcctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcgaagcttcgcggctgaaaatgacctgggtcttcctgtcgct

SEQ ID NO: 166 - DNA, 421 bp, Malus domestica
  source 1..421: mol_type = genomic DNA
cctcggagcctctggttgttgggagagtgataggagatgttcttgattccttcactgcaacaacaaaaatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcg

SEQ ID NO: 167 - DNA, 354 bp, Malus domestica
  source 1..354: mol_type = genomic DNA
aatgtctgtcacttacaacaccaagctagtctgcaatggacttgagctctttccttctgttgtcacagccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaatcaatcaacacaccttcctcgagggatcacttcagcactcg

SEQ ID NO: 168 - DNA, 246 bp, Malus domestica
  source 1..246: mol_type = genomic DNA
ccaaacctagagttgagattcaaggaggggatatgagatctttctttactttggtgatgaccgacccagattttcctggccctagtgatccttatctaagggagcacctgcactggattgtgacagacattccaggcaccacagatgccacatttggaagagaggtggtgagttatgagatgccgaagcccaacattggcatccaccggtttgtgtttgttcttttcaagcagaatcaaagacaat

SEQ ID NO: 169 - DNA, 132 bp, Malus domestica
  source 1..132: mol_type = genomic DNA
ccgacccagattttcctggccctagtgatccttatctaagggagcacctgcactggattgtgacagacattccaggcaccacagatgccacatttggaagagaggtggtgagttatgagatgccgaagccca

SEQ ID NO: 170 - DNA, 112 bp, Malus domestica
  source 1..112: mol_type = genomic DNA
ggcaccacagatgccacatttggaagagaggtggtgagttatgagatgccgaagcccaacattggcatccaccggtttgtgtttgttcttttcaagcagaatcaaagacaat

SEQ ID NO: 171 - DNA, 132 bp, Malus domestica
  source 1..132: mol_type = genomic DNA
cctggccctagtgatccttatctaagggagcacctgcactggattgtgacagacattccaggcaccacagatgccacatttggaagagaggtggtgagttatgagatgccgaagcccaacattggcatccac

SEQ ID NO: 172 - DNA, 4342 bp, Fragaria vesca
  source 1..4342: mol_type = genomic DNA
gcaatagagagaaaagaaccgtgaggaggaaaaggaggaatgaaactacttaaaaatataaagagcgaaaagaaataaagggatacgttgaaacattgggcatggtgaatgcagagagttgcagaagagatacagctagcttcggcatatgcagcagagaaaagtactttggcttcttgttatagcgaatccactccttgatatctagtgcttaattgccaattctggaacacttctctgcaacactttattcactttggcatgggttgtagcactcgtctgagatgacctctccatcagcactatcagtgggtgggttcccttttgccttgtgcttctgtctctcgttgctgaaagaagtctttgaattaccaaaataaccaagagaatctctcaaaatggtaaattcccatatgagtatacttgcttgtaaatgagaaaggttctgtggtgcaacacattcagcaaattttcttattcagttttcagtataccctatctggaaaagatgaaggttttctgttactagctgtatgaatcttcaggaagaacagagaatccgagacatttttaatttgctgtttcgttataaaatagcatgaagtgacagctaattcaaggacccggcccacatgcatgatggattgttttttttttttttttttcttttatggcaaagaacttttaagaagaacacactgtattttttttttttgaagaagaataattcagcaggtttacagccaatcaagaactgattccaaagaccatgcatattagagtacgtttggcacattagtatagaattcttatttttataaaataatgtatcatgtattaaatataaaaatttgaatagaatatcttgtattttttttgtttatatgtttctctagccacaaaattcacttgttctagctatcaataaatcacctgtttatccgattcaagaaaaagaaattcacttgtttcttacgattccttaaaatcttaaattgatgcaaggttgaaaagagtgtaataggttattgcactctgtcatttttttttatcaggtaaattacaactgtgtgagtcgaactcataacctctcacttaccaaaaagaggagagactatgttactagactaaatggcacttcatcatctttgtctgttaatttatttgttgaatatccaacatcttagttttcctgcaaggcatttcctatgtaattaaattccaagtatatatgacatcttatcattccttaagcctgatcatttcacaggtctcccaaaacttacctccttcatctttcattgtatgagtgaaaacttcccctcggcaaagccattatcgccaaaacttcttttgagtctcaagtcttaaccattctacattcatgcaagagtacgtcaacatgcatgatcgacggttgaaattgattcacagtttcgggtagtacatcttgtccagaaagaagagaacattttcctcaagaaaaagcattaaaccctttgcattgatgtgacatgcacagagagaaagagaggacgggaggtgcgagagactagctaacaatgttttaaacttctaaatttcttctaatgtcccatgatattctattcatttatactcttcttcttcttcttctctcccaaaacacttgtgagatctagagcaaactagttagcgtactgcagatcactataaatatagaccaatacagagattcacttgcaagcaaaggcaaatattagaagagctaatcctttccccgagtttatccatctttgatttatctgtacaaccttttctcttctccctctttcgagttctaacaatggcaagaatgtcggaacctctagctgttggaagagtcataggagatgttcttgattccttcacccccactacaaaaatgattctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctagctagctaggcaccttgcagggaggcaggccagatcccaaaagatctcttgctataaccagaattaatcctccctaattaaataaaagagaagtgtgtagtaatgaattaatcctccctttggctaggttattaggctaaaattactcgcatcttaccagaataattttcccaaacggagctgtacgtgagtgtttgtgtgactgtcattagcttttctttttcctctctccggcctggaatgttatgaagtaaatttgttcttagttaatttgatgtaccggtaaagtatctattcaatatagatacatgtctttggttttgaatcatcgttgatcactggaaatctataaaggaacacgtggaacaaacccattatctgtctatttcaattctcttcttctggttaaggtttagggcatccatttcatttatgatgcaattagaatctgtgttcaagctttgtccacccatggagaaaaattcaacctaggttgaatgccacatacaaatctcacccaagtgaaaaaaaacccactttcaacccataaaatgggctatctcattttccaacctaggttggattgttgggagagaagtgacccaggttggatgagaaatccagcccaggttgctttgtgacacctcaaccgagcccacttgcccatttttcacgtggtgtgcatcatcttcttgcctcccacaaacaaacgcgcttggagttggcgttttgcgcacgaaccaacaacagccatgcaatagcgcgtgtattgtgccattttgtcgagacatactgcgagcttcctaacccccccccccccccccccccgacacgtgtcacgtcaccgatgttgctaactttcattttgaatgtaacggtcaaaagatctgagccgttgctggagcaatctgactgttgtaattagctagctttttttttttgaatctgaccgttttggaatattttcaacgatgagaaaaaagtttataacggtaagaaaaaaaatatcccctataacgggaagaaaaaaatattaaaaaaatttgtataatcccctataattaaatacccatcacaattttacacatccgcaccaatttctctaagacgtcaaaatttcctagtctccaaattccgtagctttcatcttctaggttttaccttatttcaatcattctctctcttctcattcgcactcttcacaatgaataatttgtggcaaaggatccgaaaatctcaagaagaagacgactaagatgatctagcaactaacaatatcgttatcgcagccattgctcaccttgaagctgaaaaccaacctcgaggtcgcggatctcgtccaggtcgtcgtccaaaccaacctggagaaagggaggacaaaggcaaaggtatgcttgaagattattttgtcgaacgttctatttttaatgatgaggaattccgagttcgctacagaatgagccacggtgtcttcaaccgcatatgtggtgacctttgcaactacgatcgatattttgttcaaaaaatcgatgcttccaagaaggtcggtctacttccccaacaaaagatgacatcctctttacagatgctcgcctacggcacatccgcagatcaatgtgctgagtattgtcagatgacaaaatctacctctatcgagtgcctccaacgatttacaagaggaattgttgctctttactcagcagattaccttcaagctcttaatccagtcgatctaaaaaaacttcttgctaaaggtgaaaagcgaggttttccagggatgattgtgagcatcgactgcatgcattggcaatggaagaattgtccatccggtaattgggctggtgaatatagtggtagaaaacacatccccactatcattcttaaagcggtcgcattatacgacacttggatatgacacaccttctttggaatacccgggtcatgaaataacctcaatgtcctagcaaaatcctcgttgtttgacgagcttactatcggtca

SEQ ID NO: 173 - DNA, 519 bp, Fragaria vesca
  source 1..519: mol_type = other DNA
atggcaagaatgtcggaacctctagctgttggaagagtcataggagatgttcttgattccttcacccccactacaaaaatgattgtctcttacaacagcaagctcgtctgcaatggacatgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacccgccttcctcaagggatcacttcaacacccgaaccttcgcagccgaaaacgaccttggtgttcctgttgctgccgtttacttcaatgcacagagagaaacggcagcaagaagacgctag

SEQ ID NO: 174 - protein, 172 aa, Fragaria vesca
MARMSEPLAVGRVIGDVLDSFTPTTKMIVSYNSKLVCNGHELFPSAVTAKPRVEIQGGDMRSFFTLVMTDPDVPGPSDPYLKEHLHWIVTDIPGTTDATFGREVVSYEMPRPNIGIHRFVFVLFMQKRRQSVNPPSSRDHFNTRTFAAENDLGVPVAAVYFNAQRETAARRR

SEQ ID NO: 175 - DNA, 224 bp, Fragaria vesca
  source 1..224: mol_type = genomic DNA
tgagctcttcccttctgcagtcaccgccaaacctagagttgagattcaaggaggcgacatgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacatag

SEQ ID NO: 176 - DNA, 143 bp, Fragaria vesca
  source 1..143: mol_type = genomic DNA
gagattcaaggaggcgacatgagatcattcttcactctggtaatgacagacccagatgttcctggccctagtgatccttatttgaaagagcacctgcactggattgtgacagacattcctggcaccacagatgctacatttgg

SEQ ID NO: 177 - DNA, 103 bp, Fragaria vesca
  source 1..103: mol_type = genomic DNA
gagatcattcttcactctggtaatgacagacccagatgttcctggccctagtgatccttatttgaaagagcacctgcactggattgtgacagacattcctggc

SEQ ID NO: 178 - DNA, 63 bp, Fragaria vesca
  source 1..63: mol_type = genomic DNA
taatgacagacccagatgttcctggccctagtgatccttatttgaaagagcacctgcactgga

SEQ ID NO: 179 - DNA, 222 bp, Fragaria vesca
  source 1..222: mol_type = genomic DNA
tgagatcattcttcactctggtaatgacagacccagatgttcctggccctagtgatccttatttgaaagagcacctgcactggattgtgacagacattcctggcaccacagatgctacatttggaagagaagtggtgagctacgagatgccaaggccaaacataggcatccacaggtttgtgtttgttctcttcatgcaaaaacgaaggcagtcggtgaacc

SEQ ID NO: 180 - DNA, 140 bp, Fragaria vesca
  source 1..140: mol_type = genomic DNA
cctggccctagtgatccttatttgaaagagcacctgcactggattgtgacagacattcctggcaccacagatgctacatttggaagagaagtggtgagctacgagatgccaaggccaaacataggcatccacaggtttgt

SEQ ID NO: 181 - DNA, 100 bp, Fragaria vesca
  source 1..100: mol_type = genomic DNA
tttgaaagagcacctgcactggattgtgacagacattcctggcaccacagatgctacatttggaagagaagtggtgagctacgagatgccaaggccaaac

SEQ ID NO: 182 - DNA, 60 bp, Fragaria vesca
  source 1..60: mol_type = genomic DNA
ggattgtgacagacattcctggcaccacagatgctacatttggaagagaagtggtgagct

SEQ ID NO: 183 - DNA, 132 bp, Fragaria vesca
  source 1..132: mol_type = genomic DNA
cctggccctagtgatccttatttgaaagagcacctgcactggattgtgacagacattcctggcaccacagatgctacatttggaagagaagtggtgagctacgagatgccaaggccaaacataggcatccac

SEQ ID NO: 184 - DNA, 4534 bp, Arabidopsis thaliana
  source 1..4534: mol_type = genomic DNA
cggggatagagaagattcttttgggggggtttggggtcgtgactcatgtcgtaagttgttgcttgctacatcttttggattaagcttcacctaagctaaatagctagttgatacgttgtctactttttttttttttttgtcgttgatacgttgtctacttgtattgagcacattgatgtttctcttttctttttgaaaattcgatttatctttttttttttttctaaattgttaaagggttgtcttgtttggagaatttgttgtagacaactttatataagtttgatatattttgctttgtgcctttattctgattaattaccaaatcttaataaagcatcaatcaagactttctaatataagaaaataaataaattattatttagtggagaatccataataagttaataatcattatattcaataggacaaaacctttgggtgtgtttatattagtctacggattttctgatttgtataacacttttggtatttcagattaatagagtattatggttgaaatatgcattcgtaaaatcttcggaagttttaaggttttgtaactaataatcatatatagaatcccaatttgaagaatcggttcttcacgttggttttattgattcgataaaaagaagtttttttaagaatcaatgcgacaatgcgttgtcgttccacgagcaaggagtcccaaatctgcgatatgtttctttcaaatagcctctgagctaggcctgggcattcagtttcggttcggttgcgcatggtttcggttatttcggttatagtaatataagaaccatttggatatttgaggaattttggttcggttcggttcggatatctttcggttcggttcggtttggatattaaacttggtaaccataaataaactaaaaccgaaataaccgactaaaaaccgaaacaaccaaaaaatttgaccaataaccgatatagaacgtacgtaaaaaagagaaatattcacaactcacaaagatggtccatactctccataatattaaaacacctgattcacaactcacgaaattcaacttgcaaactaataaagagaaaaacatagttaagtgtcaatacatactgtgatatattatctatcatacatatatgctttggttttcatttggttatcggttattaacctaaccgaaaccgaaaccgaaatctaagacatataatattcaaccggttattttaagctatccaaacctgaaccgaaccatgtttttcggttcgattcggttcggttaatcggttagcggtttttttgcccaggcctactctgagcaataattgtatccggagttgtaatagaatcaaagtacgatgagagtgtttttatgacaaatatcttaatcttggccaattatatgttctactgaaattctttttgaattcatcgaccagtgagacttaaaaatagctttttattcgccgaggtatatatagctaggaattttgtcgaaatttagacgttagtgggttttgttcttcgtgacacaaaagatattctatatattaacgaaatctagcgatcgatatggtatttatataaagtcttggtcatagataggggttgaaacttgaaaccatgcatgatatgccaatgttgctgaagcagtcaatgttgctgaagaagtcaaacgtaattatatagtgaataccaaaaaagtgatatttcttaattcaattaaatataattatagttttaaatcacctaaaataagttacttattaaaaccccccaaatttactttaatatagttggtgtacatgtttgagaaagcaaacaaaaagaaaaagaaaaagaaaaaaaaaagagaaagaggttagtacacataattgggaattaatgtctattgattcttttatctttctctctctctctaagacggaaaacccctataaatagatgtctcggtcgtctctttgtctcccaaatcactacaaatctctcttttcctctaagttaacaaaagaaaatggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctagtttcatgattgtcataaactgcaaaaatgaaagaagaaaatttgcatgtaatctcatgtttatttgtgttctgaatttccgtactctgaataaaaactgccaaagatgagttgaatccgaaatatcaattgagtttacagaagtattgataacgatctgtcgattatcagaataaaaactagattaattgcatatcatgtttagcattgtaatactacaaaaatagtaaactcttgattaattaataaaatctaagttgctgtagtatataaatcattaaatcctcatacatggcttgataggtcacatcacatgtagtgaaccttattatatgataaacgtggagatacggaaaaggatagttaaacgatgaaaactttttttagttctggtcaaagtgacaagaccttgatgaccatctaaaatatgatcctctcctcttcttactcggcgatcatattcatatctattttcacggctggtattgtgacaaacaaccaccttttattttcaaaaaaaaatctaataggcatatgtatatttctctatagggaccctttctctatatatatatttattacattccaaaattcaacgtcaaaaatatagatttattgggtataaatccctcactagcgtagacatgtccgcggtaaacaaaaaatgtctctgcctacaaactatataattcgaaacatattagatagaaaagtcagtcttcaataaaatatttattatatgtatatatgaggtatgagatatagttttttgtcaagagttttaagaaaaaatatctatattcaaccgaataaccattaaataacaagtcttaatcattaaaatgcttacaatattttcatatttttacttaagattttactttttgctaccttgcaagttgcaaaagtataattaagtaattaacagtctagtttttgacaacaagaagaaattagcattctacattgattcagtgttcaaatggagaaaagttttttttaaatggattggccattttgggtgtataaagaatatagtacagcctgaatgatagacccatctttgagaaatgagagatgcaagtgggaaatattaatttcaacggtaaactcaagagagatcacgtgtcattaattcagtggttagcggctaagtactgttacaattatcctcaacgccgaccaacttctggttctctacatgtatattcgctatcatataaacaaatctagcaaaatacgactaacttagtaacttgtttcgattttgttagattactattacatagtttggattctcgtttattaacaaattaccaaatatcataaacatttttcgttccagctcaatgactaaaggtattgaaatcgagtttaagttcaatgtgggtttcaatcattttattttatttttgcttaagaatagtcaaagcaaaattgatcaaaaacaaactggttttggtttcattgtgaattaaaaaaaaaaagggataagataagggttatctacacatgtgccataacaaaatcttatgtaattattgcttccaagacttcagagctaaaataaataaagaaaaaactgaagaaaaaacaaagaagtatatacagtacacctacgtaggcaaattgattcgctaccaaatcaagggcatgcaaagactttaggatttgcctgaaaaagacactgtgaactagaacattatatagagttttagggtttcagtaacacataatgtgcctgtgttgtgtttagccacttatggttattttcatagctaaatatttttccaaaagtgaaacaccatttttatttataaaatgactggcccctcgcgtttggtgttattacaaatcaacacgtgctctgttccattccatccctgattttactcaaaattttgctacatctaaatatatttcttattgccaaaattgagatggtgattggtgtcagtatttgggtatgaatcgttttgaactaaaagtctaaaacattat

SEQ ID NO: 185 - DNA, 534 bp, Arabidopsis thaliana
  source 1..534: mol_type = other DNA
atggagaatatgggaactagagtgatagagccattgataatggggagagtggtaggagatgttcttgatttcttcactccaacaactaagatgaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagtatgatcttggtctccctgtcgcggccgtcttctttaacgcacaaagagaaaccgctgcacgcaaacgctag

SEQ ID NO: 186 - protein, 177 aa, Arabidopsis thaliana
MENMGTRVIEPLIMGRVVGDVLDFFTPTTKMNVSYNKKQVSNGHELFPSSVSSKPRVEIHGGDLRSFFTLVMIDPDVPGPSDPFLKEHLHWIVTNIPGTTDATFGKEVVSYELPRPSIGIHRFVFVLFRQKQRRVIFPNIPSRDHFNTRKFAVEYDLGLPVAAVFFNAQRETAARKR

SEQ ID NO: 187 - DNA, 371 bp, Arabidopsis thaliana
  source 1..371: mol_type = genomic DNA
gaatgttagttataacaagaagcaagtctccaatggccatgagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagt

SEQ ID NO: 188 - DNA, 331 bp, Arabidopsis thaliana
  source 1..331: mol_type = genomic DNA
gagctctttccttcttctgtttcctccaagcctagggttgagatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatcccttcgagagatcacttcaacactcgtaaatttgcggtcgagt

SEQ ID NO: 189 - DNA, 252 bp, Arabidopsis thaliana
  source 1..252: mol_type = genomic DNA
agatccatggtggtgatctcagatccttcttcactttggtgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttcaggcagaagcaaagacgtgttatctttcctaatatccctt

SEQ ID NO: 190 - DNA, 174 bp, Arabidopsis thaliana
  source 1..174: mol_type = genomic DNA
tgatgatagacccagatgttccaggtcctagtgacccctttctaaaagaacacctgcactggatcgttacaaacattcccggcacaacagatgctacgtttggcaaagaggtggtgagctatgaattgccaaggccaagcatagggatacataggtttgtgtttgttctgttca

SEQ ID NO: 191 - DNA, 136 bp, Arabidopsis thaliana
  source 1..136: mol_type = genomic DNA
tccaggtcctagtgacccctttctaaaagaacacctgcactggatcgttacaaacattcccggcacaacagatgctacgtttggcaaagaggtggtgagctatgaattgccaaggccaagcatagggatacatagg

SEQ ID NO: 192 - DNA, 132 bp, Arabidopsis thaliana
  source 1..132: mol_type = genomic DNA
ccaggtcctagtgacccctttctaaaagaacacctgcactggatcgttacaaacattcccggcacaacagatgctacgtttggcaaagaggtggtgagctatgaattgccaaggccaagcatagggatacat

SEQ ID NO: 193 - protein, 44 aa, Rubus occidentalis
PGPSDPYLKEHLHWVVTDIPGTTDATFGREVVSYEMPRPNIGIH

SEQ ID NO: 194 - protein, 44 aa, Pyrus pyrifolia
PGPSDPYLREHLHWIVTDIPGTTDATFGREVVSYEMPKPNIGIH

SEQ ID NO: 195 - protein, 44 aa, Prunus serotina
PGPSDPYLREHLHWIVTDIPGTTDATFGREVVSYEMPRPNIGIH

SEQ ID NO: 196 - protein, 44 aa, Fragaria vesca
PGPSDPYLKEHLHWIVTDIPGTTDATFGREVVSYEMPRPNIGIH

SEQ ID NO: 197 - protein, 44 aa, Arabidopsis thaliana
PGPSDPFLKEHLHWIVTNIPGTTDATFGKEVVSYELPRPSIGIH

SEQ ID NO: 198 - DNA, 23 bp, synthetic construct
  source 1..23: mol_type = other DNA
gtagttggggtgaaggaatcaag

SEQ ID NO: 199 - DNA, 23 bp, synthetic construct
  source 1..23: mol_type = other DNA
gtggtgactgcagaaggtaagag

SEQ ID NO: 200 - DNA, 23 bp, synthetic construct
  source 1..23: mol_type = other DNA
atttgtaggtgatgacagaccca

SEQ ID NO: 201 - DNA, 23 bp, synthetic construct
  source 1..23: mol_type = other DNA
aaggagcacctgcactggtataa

SEQ ID NO: 202 - DNA, 23 bp, synthetic construct
  source 1..23: mol_type = other DNA
taggtgatgacagacccagatgc

SEQ ID NO: 203 - DNA, 23 bp, synthetic construct
  source 1..23: mol_type = other DNA
attgtaagttataccagtgcagg

SEQ ID NO: 204 - DNA, 23 bp, synthetic construct
  source 1..23: mol_type = other DNA
gttattttcacaggtaatgacag

SEQ ID NO: 205 - DNA, 23 bp, synthetic construct
  source 1..23: mol_type = other DNA
acaggtaatgacagacccagatg

SEQ ID NO: 206 - DNA, 23 bp, synthetic construct
  source 1..23: mol_type = other DNA
aaataaggatcactagggccagg

SEQ ID NO: 207 - DNA, 23 bp, synthetic construct
  source 1..23: mol_type = other DNA
ttgttggggtgaaacaatcaaga

SEQ ID NO: 208 - DNA, 23 bp, synthetic construct
  source 1..23: mol_type = other DNA
gtggtgacagcagaaggatagag

SEQ ID NO: 209 - DNA, 23 bp, synthetic construct
  source 1..23: mol_type = other DNA
ttcaccccaactaccaaaatgat

SEQ ID NO: 210 - DNA, 23 bp, synthetic construct
  source 1..23: mol_type = other DNA
ttagagacgtgcctcgtagtgta

SEQ ID NO: 211 - DNA, 20 bp, synthetic construct
  source 1..20: mol_type = other DNA
ggcaagaatgtcggaaccct

SEQ ID NO: 212 - DNA, 20 bp, synthetic construct
  source 1..20: mol_type = other DNA
ggcccaacatcggcatccac

SEQ ID NO: 213 - DNA, 20 bp, synthetic construct
  source 1..20: mol_type = other DNA
ggcaccacagatgctacatt

SEQ ID NO: 214 - DNA, 5843 bp, synthetic construct
  source 1..5843: mol_type = other DNA
ccaccatatccaaatctctaaaccytaaaccytaaaatycaccatacacctaatccctcgaccctarayycaaaatyyacyataccyctaatytttcaaycctaaacgycacaaatcattaaycaaaaaytmtaaactctaaytcctcaaccttaacccttaaaytgtaaattytaaactcataaccttaaattytaaaccctaaatcctaaacgtaaatcccccaaaayyctagctacaagtaattyctcaagtcttcytaattawtgatyatgcyyaaattgggacatattacttgatatcttctcaactargttgttatctactaacatgccttatcatttgcactaaatraatatatgaccayattctttcctaaatycaaattactaagcattatcaatttrctatttagttgtctttrttttytcyatgatgatraggtagtaacattgtcatstraatcraastttagtgaaagtaartgatttgaktcgtttacaatcaattaaacaatagttctttcatgtggttataawcaatwrttccataattgtaatctcaattaaarttcatctaaattrtacctcctttcyaattaagtttacattgctaagtttttttttttataatatatatatatatatatatatattaaagtgcatgattctatttgattgataaaaaatatatttataaagataaaattataattgtaaggtaattcattrataaratrtttatatataaagctattaattataattgtaagtataattaattaacaagttttaaagttgagtaagcaatagccattgaatctgagatgagtatgatagactttgcataaaatgastaggcaattgtttaatgatgtgtatatgatgtggcatgccasatatgattgaracctcyacatcttaggtcttagagcatytycaasagttttcctaaaaaatctttattatagaaattcaaaagttaaaatwtctataatttttattttgcarttccaayagtttttctatcatattctctataatttattatctacaattaaatatggtattatttttaaatttaaaaagtataatcaaataattaatratgaaaatgaatatatttggaaagaggaaagagaakagggtaaaatttttyaaaaagatgtttcyctataattttagagtttgctrtaaatatagagattyttattttctctcttctcactatctctaaaatagagatargtatagggaarctattggagtaaaaaaatactcaatttttcctaaactrgagaaaaaataaattatagggaagctcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaacgtattwttgctacaagaaaactawgtcatttttgaaaacaaaacctgkatttgtgaagatatcaagcacaattttttttttctttttgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcaatgcttgtytccraatttgaagaaaaytttagttttttttttttgttacattggagggratgaacctytracctagyctaattctaaytcaattaytcaccaacccatactayttgtgcyaactctaagggcttggaaaacattagtttcttgaatatgtrtgtktatatatataaatatatatatatagatattaatgtgaatttgttttctyttcattacatggtyatttatgtttttggaattttagaatataattgratctagaaattaaagcaagaaaaaagtgcttatgaaaatctgaactagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctctcyttcgcactcttcttctagtttaagtttagggttttggtktccwtttcatttsaacgggaaaatcagaaggaatcaacrcgatgacccaatctagtctgcgtgcgggccccacactgtcatgtccgatcttaaataactaattaaccgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgacaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttaaaattcgttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctatcggagatggagggtgcggtggtgatccgctaggctcaggagcagcagcggagagcagtgatgggattrcaactgcggctcgagcackggcycgakgacgacrycgrygrcrtgagatgggtgcagagagaagagaagaaaggaaaaggaaaagaagagaaattcggtgcccgaggcgaactgaaattttaaggcctaattttttctatataaatgtatacatttatttttagtataatttttttcattaaatagagaaatgcgtaattattyaattaatcagagtaatttgtggagagtatgaaaaaatttaagacatytttgaaatcgtgaaaattaatacttaagttgaattcttaaatctcttcattaattt

SEQ ID NO: 215 - DNA, 1113 bp, synthetic construct
  source 1..1113: mol_type = other DNA
aggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccatctttgatttgcctttacgactctctctctmtctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtgatgacagacccagatkctcctggccctagtgatccttatttgaaggagcacctgcactgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggt

SEQ ID NO: 216 - protein, 172 aa, synthetic construct
MARMSEPLVVGRVIGDVLDSFTPTTKMIVTYTTRHVSNGYELLPSAVTTKPRVEIQGGDMRSFFTLVMTDPDXPGPSDPYLKEHLHWVVTDIPGTTDATFGREVVSYEMPRXNIGIHRFVFVLFRQKRRQSVNPPXSRDXFNTRSFAAENDLGLPVAAVYFNXQRETAARRR

SEQ ID NO: 217 - DNA, 295 bp, synthetic construct
  source 1..295: mol_type = other DNA
tctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgt

SEQ ID NO: 218 - DNA, 215 bp, synthetic construct
  source 1..215: mol_type = other DNA
gtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 219 - DNA, 175 bp, synthetic construct
  source 1..175: mol_type = other DNA
gtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatca

SEQ ID NO: 220 - DNA, 135 bp, synthetic construct
  source 1..135: mol_type = other DNA
gaagggtcattggagatgttcttgattccttcaccccaactaccaaaatgattgtcacttacactacgaggcacgtctctaatggatatgagctcttaccttctgcagtcaccaccaaacctagagttgagattc

SEQ ID NO: 221 - DNA, 95 bp, synthetic construct
  source 1..95: mol_type = other DNA
cttgattccttcaccccaactaccaaaatgattgtcacttacactacgaggcacgtctctaatggatatgagctcttaccttctgcagtcaccac

SEQ ID NO: 222 - DNA, 223 bp, synthetic construct
  source 1..223: mol_type = other DNA
tctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggt

SEQ ID NO: 223 - DNA, 143 bp, synthetic construct
  source 1..143: mol_type = other DNA
gtcgtaawaatggcaagaatgtcggaacccttggttgttggaagggtcattggagatgttcttgattccttcaccccaactaccaaaatgattgtcacttacactacgaggcacgtctctaatggatatgagctcttaccttc

SEQ ID NO: 224 - DNA, 103 bp, synthetic construct
  source 1..103: mol_type = other DNA
gtcggaacccttggttgttggaagggtcattggagatgttcttgattccttcaccccaactaccaaaatgattgtcacttacactacgaggcacgtctctaat

SEQ ID NO: 225 - DNA, 63 bp, synthetic construct
  source 1..63: mol_type = other DNA
gaagggtcattggagatgttcttgattccttcaccccaactaccaaaatgattgtcacttaca

SEQ ID NO: 226 - DNA, 223 bp, synthetic construct
  source 1..223: mol_type = other DNA
ggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgt

SEQ ID NO: 227 - DNA, 142 bp, synthetic construct
  source 1..142: mol_type = other DNA
accccaactaccaaaatgattgtcacttacactacgaggcacgtctctaatggatatgagctcttaccttctgcagtcaccaccaaacctagagttgagattcaaggaggtgacatgagatcattcttcactctggtataca

SEQ ID NO: 228 - DNA, 103 bp, synthetic construct
  source 1..103: mol_type = other DNA
tgtcacttacactacgaggcacgtctctaatggatatgagctcttaccttctgcagtcaccaccaaacctagagttgagattcaaggaggtgacatgagatca

SEQ ID NO: 229 - DNA, 63 bp, synthetic construct
  source 1..63: mol_type = other DNA
acgtctctaatggatatgagctcttaccttctgcagtcaccaccaaacctagagttgagattc

SEQ ID NO: 230 - DNA, 220 bp, synthetic construct
  source 1..220: mol_type = other DNA
gagatcattcttcactctggtgatgacagacccagatkctcctggccctagtgatccttatttgaaggagcacctgcactgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaac

SEQ ID NO: 231 - DNA, 140 bp, synthetic construct
  source 1..140: mol_type = other DNA
cctggccctagtgatccttatttgaaggagcacctgcactgggttgtgacagacattccaggcaccacagatgctacatttggaagagaggtggtgagctatgagatgccaaggccyaacatcggcatccacaggtttgt

SEQ ID NO: 232 - DNA, 100 bp, synthetic construct
  source 1..100: mol_type = other DNA
tttgaaggagcacctgcactgggttgtgacagacattccaggcaccacagatgctacatttggaagagaggtggtgagctatgagatgccaaggccyaac

SEQ ID NO: 233 - DNA, 60 bp, synthetic construct
  source 1..60: mol_type = other DNA
gggttgtgacagacattccaggcaccacagatgctacatttggaagagaggtggtgagct

SEQ ID NO: 234 - DNA, 5840 bp, synthetic construct
  source 1..5840: mol_type = other DNA
ycaccatayccaaatctctaaaccytaaaccctaaaattcaccataccyctaatcyctcraccctaratacaaaatccacyatacyyctaatycctcaaycctaaacgycacaaatcattaaycaaaaactctaaactctaaytcctcaaycttaaccyttaaaytgtaaattywaaactcataaccttaaattytaaaccctaaatmctaaacgtaaccyccctaaaayyctarctacaagtaattyctcaagtcttcytaattawtratyaygcyyaaattgggacatattayttgatatcttctcaactrggttgttatctactaacatgccttatcatttgyaytamayraatatatgaccayatyctttcmtaaatycaaawtactaarcattwtcaatttgctatttagttgtctttrttttctccatgatgatragrtagtaacattgtcaygtgaatcraagtttagtgaaatcaaatratttgattcgtttgyaatyaattamacaatagttctttcatgtggttataaacaatagttccataattgtaatctcaattaaagttcatctaaattatayctcctttccaattaagtttakattgctargtttttttttttataatatatatatatatatatatatattamagtgcatgattctatttgattgataaaaaatatatttataaagataaaattataattgtaaggtaattcattgataaratatttatatstaaarctattaattataattgtaagtataattaattrayaagttttaaarttgagtaagcaatagycattgaatctdagatgagtwtratagactnttgcmtaaaatgagtannnnattkyttaatgatgtgtatatgatgtggyatgcyatatatgattgagacctccacatcttaggtcttagagcatctycaabagttttcctaaaaaatctttattatagrgaytcaaaagttaaaatytctatartttttcttycatarttccaacagtttttctatcatattctctataatttattatctacaattaaatatggtattatttttaaatttaaaaagtataatcaaataattaatgatgaaaatgaatatatttggaaagaggaaagagaakagggtaaaawtttccaaaaagatgtttcyctataattttagagtttgctrtaaatatagagatttttrttttctctctyctcactatctctaaaatagagataggtatagrraarctattggagtaaaaaaatacwcaatttttcctaaactagagaaaaaataaattatagggaagctyttggagatgctcttaatgaagtcaacratcyttttccattcagcatacatgymataccttatagcctcatttcacatatatttcacccaaaacttrcctccttaattacctctttgsgtagtactagagggtctcaattaasgcctaactttygaggtctcaattaaaaygtaatttytgatgccycaatcycatatagcggtccatgtcatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattgttaaagtgttgtttmaaaaatggtgtaccaaamgtattwttgctacaagaaaacwawgtcatttttgaaaacaaaacctgkatttgtgaagatatcaarcrcaannttttttttttctttttgaaaaccaacacgtactaagagtcatgcaagagtcaacataatcaatggttcgatttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcaacacacttasagatctaaagcaagtactggttagcgaacttgaaattactataaatatagagcaatacagggaaaggcaattataagaagtacaaatgcctcctccttcctgagttwtcagttttcttatccatctttgatttacctttacgactctctctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtgtkktktcagatctacgaaatcgcttttaagttaattraatttgatttgtaggtgatgacagacccagatkctcctggccctagtgatccttatttgaaggagcacctgcactggtataacttacaatcaaacctagaaactaaaaaaacaaagacaataaacattcaattaattaaattcagaattaattataaatgttgtcacattcagtkcgttcaattcggatttagagtctaaaaacawaagcaatgcttgtytccraatttgaagaaaayattagtttttttttttrttacattggagggawtgaacctttaacctagtctaattctaattcaattactcaccaacccataytayttrtgcyaactctaagggmttggaaaacattagtttcttgaatatgtgtgtgtatatatataaatatatatatatagatattaatgtgaatttsttttctyttcattacatggtcatatttatgtttttggaattttagaatataattgratctagaaattaaagcaagaaaaaagtgcttatgaaaatctgaactagaaaagctytacaattatggagtatatacatatcmagctgggagctaacagttttggtttaaattgaactaaacagggttgtgacagacattccaggcaccacagatgctacatttggttggttaattcactgctagttcagctaccagttatttagtgatcttatagctagagaagatatatcagacaatatcagttatcratcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcacttcaacacccgaarcttcgcagccgaaaatgacctcggtcttcctgtcgctgccgtttacttcaatgcrcagagagaaacagcagcgagaagacgctagctagctagccgcaggaggcaggccagacycccaaatttccaggctataaccatartatctaattaaataaaagaaaagggtggttaatgatttaatcctccctattgttaggttcctaggctaaaagtacttaakttcccaragagagcygtgagtgtttgtgcgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctstcyttcgcactcttcttstagtttaagtttagggttttggtktccatttcatttsaacargaaaatcakaaggaatcaacrcgatgacccaatctagtctgcgtgcgggcyccacactgtcatgtccgatcttaaataactaattaaccgttggtattgtatattcaaaagtaagtggaaaaatattaattatgttagatgctatatcaataaactttagtagsacraaaaaataaattaaaaatgaaacacaacatcattttgacatgacacaagcatgatakttacattacaattaaagcaacaaattcatattatgtcaatgttttttttacatttctcgttcacaaagtatttttttttttgagaaaataacataaaytttattaattaatataaaaggattacaatcctaagcatgaacatcytaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgayaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttawaattcgttaacatttagaaagaaataccactaaattgatgcttagttartatacaatttctaattccacttggttagatwatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggaagggaagagagmgctgggttcgtgaggtkgctrtcggagatggagstgcrgcggtgatmmgctaggctcaggaggagtagcggagagcagtgatgggattrcarstgmggctcgagcackggctcgaggacgacrccgrcggcgtgagatgggtgcagagagaagagaagaaaggaaaaggaaaagaagagaaattcggtggccgaggcgaactgaaattttaagryctaattttttctatrtaaatgtatacatttatttttggtataannnnnnnnttywttwmattraatagaraaatgcrtaattattyaattaatyrgagtaatttgtggagartatgaaaaaatttaagayatmtttraaatygtraaaattaatacttaagttgaattcttaaatctcttcattaattt

SEQ ID NO: 235 - DNA, 522 bp, synthetic construct
  source 1..522: mol_type = other DNA
atggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtgatgacagacccagatkctcctggccctagtgatccttatttgaaggagcacctgcactggtgggttgtgacagacattccaggcaccacagatgctacatttggaagagaggtggtgagctatgagatgccaaggccyaacatcggcatccacaggtttgtgtttgttctcttcaggcagaaacgaaggcagtcagtgaaccctccttcmtcaagggatcacttcaacacccgaarcttcgcagccgaaaatgacctcggtcttcctgtcgctgccgtttacttcaatgcrcagagagaaacagcagcgagaagacgctag

SEQ ID NO: 236 - protein, 173 aa, synthetic construct
MARMSEPLVVGRVIGDVLDSFTPTTKMIVTYTTRHVSNGYELLPSAVTTKPRVEIQGGDMRSFFTLVMTDPDXPGPSDPYLKEHLHWWVVTDIPGTTDATFGREVVSYEMPRXNIGIHRFVFVLFRQKRRQSVNPPXSRDHFNTRXFAAENDLGLPVAAVYFNXQRETAARRR

SEQ ID NO: 237 - DNA, 295 bp, synthetic construct
  source 1..295: mol_type = other DNA
tctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtg

SEQ ID NO: 238 - DNA, 215 bp, synthetic construct
  source 1..215: mol_type = other DNA
gtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 239 - DNA, 223 bp, synthetic construct
  source 1..223: mol_type = other DNA
tctctctctctctctctctctctctctctctctctcgtaagtcgtaataatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggt

SEQ ID NO: 240 - DNA, 143 bp, synthetic construct
  source 1..143: mol_type = other DNA
gtcgtaataatggcaagaatgtcggaacccttggttgttggaagggtcattggagatgttcttgattccttcaccccaactaccaaaatgattgtcacttacactacgaggcacgtctctaatggatatgagctcttaccttc

SEQ ID NO: 241 - DNA, 223 bp, synthetic construct
  source 1..223: mol_type = other DNA
ggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatctttttttttttgtctgggttcgtytttttaccaattgtg

SEQ ID NO: 242 - DNA, 143 bp, synthetic construct
  source 1..143: mol_type = other DNA
accccaactaccaaaatgattgtcacttacactacgaggcacgtctctaatggatatgagctcttaccttctgcagtcaccaccaaacctagagttgagattcaaggaggtgacatgagatcattcttcactctggtatacat

SEQ ID NO: 243 - DNA, 221 bp, synthetic construct
  source 1..221: mol_type = other DNA
aactagaaaagctytacaattatggagtatatacatatcmagctgggagctaacagttttggtttaaattgaactaaacagggttgtgacagacattccaggcaccacagatgctacatttggttggttaattcactgctagttcagctaccagttatttagtgatcttatagctagagaagatatatcagacaatatcagttatcratcaatwtwtattc

SEQ ID NO: 244 - DNA, 140 bp, synthetic construct
  source 1..140: mol_type = other DNA
agctgggagctaacagttttggtttaaattgaactaaacagggttgtgacagacattccaggcaccacagatgctacatttggttggttaattcactgctagttcagctaccagttatttagtgatcttatagctagaga

SEQ ID NO: 245 - DNA, 100 bp, synthetic construct
  source 1..100: mol_type = other DNA
ggtttaaattgaactaaacagggttgtgacagacattccaggcaccacagatgctacatttggttggttaattcactgctagttcagctaccagttattt

SEQ ID NO: 246 - DNA, 60 bp, synthetic construct
  source 1..60: mol_type = other DNA
gggttgtgacagacattccaggcaccacagatgctacatttggttggttaattcactgct

SEQ ID NO: 247 - protein, 42 aa, synthetic construct
PSDPYLKEHLHWVVTDIPGTTDATFGREVVSYEMPRXNIGIH

SEQ ID NO: 248 - protein, 43 aa, synthetic construct
PSDPYLKEHLHWWVVTDIPGTTDATFGREVVSYEMPRXNIGIH

SEQ ID NO: 249 - DNA, 23 bp, synthetic construct
  source 1..23: mol_type = other DNA
gtagttggggtgaaggaatcaag

SEQ ID NO: 250 - DNA, 23 bp, synthetic construct
  source 1..23: mol_type = other DNA
gtggtgactgcagaaggtaagag

SEQ ID NO: 251 - DNA, 20 bp, synthetic construct
  source 1..20: mol_type = other DNA
ggcaccacagatgctacatt

SEQ ID NO: 252 - DNA, 5843 bp, synthetic construct
  source 1..5843: mol_type = other DNA
ccaccatatccaaatctctaaaccytaaaccytaaaatycaccatacacctaatcyctcgaccctarayycaaaatyyacyatacyyctaatytttcaaycctaarygycacaaatcattaaycaaaaaytmtaaaytctaaytcctcaaycwtaacyyttaaaytrtaaattytaaactcataaccttaaattytaaaccctaaatcctaaacgtaaatcccccaaaayyctarctacaartaattyctcaagtcttcytaattawtratyayrcyyaaattgggayatattacttratatcttctcaactargttgttatctactaacatgccttaycatttgyamtamatraatatatgaccayatyctttcctaaatycaaattaytaagcattatcaatttrctatwtagttgtctttrttttytcyatgwtgatraggtagtaacattgtcatstraatcraastttagtgaaagtaartgatttgaktygtttacaatcaattaaamaatagttctttcatgtggttataawyaatwrttccatrattgtaatctcaattaaarttcwtcwaaattrtacctcmtttcyaattaagtttacattgctaagtttttttttttataatatatatatatatatatatatattaaagtgcatgattctatttgattgataaaaaatatatttataaagataaaattataattgtaaggtaattcattrataaratrtttatatataaarctattaattataattgtaagtataattaattaacaagttttaaarttgagtaagcaatagccattgaatctgagatgagtatgatagactttgcataaaatgastaggcaattgtttaatgatgtgtatatgatgtggyatgccasatatgattgaracctcyacatcttaggycttagagcatytycaasagtttycytaaaaaatctttattatagaaattcaaaagttaaaatwtctawawtttttattttgcarttccaayagtttwtctatcatattctctataatttattatmtacaattaaatatggtattatttttaaatttwaaaartataatcaaataattaatratgraaatgaatatatttggaaagargaaagaraakagrgtaaaawtttyyaaaaasatrtttcyctataattttagagtttgctrtaaatatarakattyttattttctytcttctcactawctctaaaatagagatargtatagggaarytattggagyaaaaaaatactcaattwttcctaaactrgagaaaaaataaattatagggaarstcttggagatgctcttaatgaagtmaacratcyttttccawtcagcatacatgymataccttatagcctcatttcacatatatttcacccaaaacttacctccttaattacctctttgggtagtactagagggtctcaattaaggcctaacttttgaggtctcaattaaaacgtaatttytgatgccyyaatcycayatagcggtccatgtyatattcatgtcatcatttttatygcatcatattttgcaatacctattttactatttttattttgtaattagattgtactcatttatattaaaaatacacaaaaaaaaaattcctagaaaactggtgtatttatgtcaatatatgcaaatgctatatcatttttggtgtttttagataattkttaaagtgttgtttmaaaaatggtgtaccaaamgtattwttgctacaagaaaactawgtcatttttgaaaacaaaacctgkatttgtgaagatatcaagcacaatttwttwtwtcttttkgaaaaccaacacgtactaagagtcatrcaagagtcaacataatcaatggttygawttgatagttttataacatttctgtcmataaagaagagaacattttcctctagaaaaagcattgaaccccttgcatcgatgtgacatgcasacagagagagaaaggatgggaggtgggggactaacaatgttttaatttctaatgyccatgatattctattcatttatactcttcttcttattgctctctttcracacacttagagatctaaagcaagtactggttagcgaacttgaaattactataaatatagagcaatacagggaaaggcaattataagaagtacaaatgcctcctccttcctgagttwtcagttttcttatccrtctttgatttgcctttacgactctctctctmkctctctctctctctctctctctctctctctckctmtctctctctctctcgtaagtcgtaawaatggcaagaatgtcggaacccttggttgttggaagggtcattggagatgttcttgattccttcaccccaactaccaaaatgattgtcacttacactacgaggcacgtctctaatggatatgagctcttaccttctgcagtcaccaccaaacctagagttgagattcaaggaggtgacatgagatcattcttcactctggtatacatcttttttttttgtctgggttcgtyttttwaccaattgtgtkttwtcagatctacgaaatcgcttttaagttaattgaatttgatttgtaggtgatgacagacccagatkctcctggccctagtgatccttatttgaaggagcacctgcactggtataacttacaatcaaacctagaaactaaaaaaacaaagacaatamacattcaattaattaaattcagaattaattataaatgttgtcacattcagttcgttcaattcggatttagagyctaaaaacataagcwatgcttgtytcyrratttgaagaaaaytttagtttttttttttkrttacattggarggrwtgaacctytracctagyctaattctaaytcaattaytcaccaacccatactayttgtgcyaactctaagggmttggaaaacattagtttcttgaatatgtrtgtktatatatataaatatatatatatagatattaatgtgaatttgttttctyttcattacatggtyatttatgtttttggaattttagaatataattgratytagaaattaaagcaagaaaaaagtgcktatgaaaatctgaacwagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtattctggttctaatttgattaaattgtatacgtaacaggaagagaggtggtgagctatgagatgccaaggccyaacatcggcatccacaggtttgtgtttgttctcttcaggcagaaacgaaggcagtcagtgaaccctccttcmtcaagggatcayttcaacacccgaagcttcgcagccgaaaatgacctcggtcttcctgtcgctgccgtttacttcaatgcrcagagagaaacagcagcgagaagacgctagctagctagccgcaggaggcaggccagacycccaaatttccaggctataacyataatatctaattaaataaaagaaaagggtggttaatgatttaatcctccctattgttaggttcttaggctaaaagtacttaakttcccaaagagagcmgtgagtgtttgtgtgactctcatttgatttttgctctcaccatcatcgaatggttataagtttgttctgacttctgatcagttcaatgtatytgtaaagcatctatattcgtatattatatgsggttcttgtttttggttttgaatcgttgmtgatcaactggaactgtaaagggacacgtggaacaaacccattatctstcyttcgcactcttcttctagtttaagtttagggttttggtktccwtttcatttsaacgggaaaatcakaaggaatcaacrcgatgacccaatctagtctgcgtgcgggccccacactgtcatgtccgatcttaaataactaattaacsgttggtattgtatattcaaaagtaagtggaaaaatattaattatgttagatgctatatcaataaactttagtagcacraaaaaataaattaaaaatgawacacaacatcattttgacatgacacaagatgatakttacattacaattaaagcaacaaattcatattatgtcaatgtttttttacatttctcgttcacaaagtatttttttttttgagaaaatataaaytttattaattaatataaaaggattacaatcctwagcatgaacatcctaagaaagagcattatagaccacacaaatgacctaatcgatgagtctaaacctagaactctaatcgatgattcaaaacctagaacaatacatacaccgtgcattactgctatcatacaaagtatttatgtgaacgttattgatcatatgttatccaaaggatgattacggttttcatttctaaatgccttaatagttttgttcgayaaactaaaccttgagacttttttaaaaaaaaattccaacatttttttttatcaaggataaatattcattaataattttggtcaattacaaccatggggcagagtggcttattttgaagagatgccacacccctaaaatcaactagagacatcatcagaaagtaatttcttcaaccaagggggaccaacttctttccaaaaaactggcccaagaataggaattgcaaccttagcaagagcatgagccacaccattacacaatctcggaatcttcttgaattttatttcctccacttgagttgttatcaacttgatttcttcaaagatatggccttcaggattcagatcccagttattagcttcaattgtcttgatcaccaatagagcatcgcttttaattttaatttttcgaaaaccttccttgatgcaacacttcaacccaaaaagaagagcttgtgcttctgcagcaaacacatggagggaaccagtaatagcttccatcatggcgccaactaaatcgcctttatcatttctgattactacccccagtccagtgtgctgatgtcgagtatcacacgcggcatctacatcatggcgccaacatttysttttamaattcrttaacatttagaaagaartaccactaaattgatgcttwgttartatacaatttctaaatccacktggttagattatagtaattatactatatactaaagtttctagcaccgttaattaatattcataacgaaattacgattatatcctcatttagtcataaattacgattttaccattactcttttttttttcttcccccctcactctttttcttccttcttttttgtttttttttttccttttcttctacgtcaggargggaagagagmgctgggttcgtgaggtwgctrtcggagatggagggtgcggtggtgatccgctaggctcaggagcagcagcggagagcartgatrggattrcaactgcggctckagcackggcycgakgacgacrycgrygrcrtgagatgggtgcagagagaagagaagaaaggaaaaggaaaagaagagaaattcggtgcccgaggcgaactgaaattttaaggcctaattttttctatataaatrtatacatttattttkrgtataatttttttcattaaatagaraaatgcgtaattattyaattaatcagartaatttgtgkagagtatgaaaaaatwtaagayatytttraaatyrtraaaattaatayttaagtwraattyttaaatytctwcattaattt

SEQ ID NO: 253 - protein, 172 aa, Rubus sp.
MARMSEPLVVGRVIGDVLDSFTPTTKMIVTYTTRHVSNGYELLPSAVTTKPRVEIQGGDMRSFFTLVMTDPDXPGPSDPYLKEHLHWVVTDIPGTTDATFGREVVSYEMPRPNIGIHRFVFVLFRQKRRQSVNPPSSRDHFNTRSFAAENDLGLPVAAVYFNAQRETAARRR

SEQ ID NO: 254 - DNA, 295 bp, synthetic construct; identical to SEQ ID NO: 217
  source 1..295: mol_type = other DNA

SEQ ID NO: 255 - DNA, 215 bp, synthetic construct; identical to SEQ ID NO: 218
  source 1..215: mol_type = other DNA

SEQ ID NO: 256 - DNA, 175 bp, synthetic construct; identical to SEQ ID NO: 219
  source 1..175: mol_type = other DNA

SEQ ID NO: 257 - DNA, 135 bp, synthetic construct; identical to SEQ ID NO: 220
  source 1..135: mol_type = other DNA

SEQ ID NO: 258 - DNA, 95 bp, synthetic construct; identical to SEQ ID NO: 221
  source 1..95: mol_type = other DNA

SEQ ID NO: 259 - DNA, 223 bp, synthetic construct; identical to SEQ ID NO: 222
  source 1..223: mol_type = other DNA

SEQ ID NO: 260 - DNA, 143 bp, synthetic construct; identical to SEQ ID NO: 223
  source 1..143: mol_type = other DNA

SEQ ID NO: 261 - DNA, 103 bp, synthetic construct; identical to SEQ ID NO: 224
  source 1..103: mol_type = other DNA

SEQ ID NO: 262 - DNA, 63 bp, synthetic construct; identical to SEQ ID NO: 225
  source 1..63: mol_type = other DNA

SEQ ID NO: 263 - DNA, 223 bp, synthetic construct; identical to SEQ ID NO: 226
  source 1..223: mol_type = other DNA

SEQ ID NO: 264 - DNA, 143 bp, synthetic construct; identical to SEQ ID NO: 242
  source 1..143: mol_type = other DNA

SEQ ID NO: 265 - DNA, 103 bp, synthetic construct; identical to SEQ ID NO: 228
  source 1..103: mol_type = other DNA

SEQ ID NO: 266 - DNA, 63 bp, synthetic construct; identical to SEQ ID NO: 229
  source 1..63: mol_type = other DNA

SEQ ID NO: 267 - DNA, 220 bp, synthetic construct
  source 1..220: mol_type = other DNA
aacwagaaaagctytacaattatggagtatatacatatcmagctgggarctaacagttttggtttaaattgaactaaacagggttgtgacagacattccaggcaccacagatgctacatttggttggttaattcactgctagttcagctaccagttatttagtgatcttatagctagagaagatatatcagayaatatcarttatcgatcaatwtwtatt

SEQ ID NO: 268 - DNA, 140 bp, synthetic construct
  source 1..140: mol_type = other DNA
agctgggarctaacagttttggtttaaattgaactaaacagggttgtgacagacattccaggcaccacagatgctacatttggttggttaattcactgctagttcagctaccagttatttagtgatcttatagctagaga

SEQ ID NO: 269 - DNA, 100 bp, synthetic construct; identical to SEQ ID NO: 245
  source 1..100: mol_type = other DNA

SEQ ID NO: 270 - DNA, 60 bp, synthetic construct; identical to SEQ ID NO: 246
  source 1..60: mol_type = other DNA

SEQ ID NO: 271 - protein, 41 aa, Rubus sp.
MARMSEPLVVGRVIGDVLDSFTPTTKMIVTYTTRHVSNGYE

SEQ ID NO: 272 - protein, 27 aa, Rubus sp.
LVVGRVIGDVLDSFTPTTKMIVTYTTR

SEQ ID NO: 273 - protein, 41 aa, Rubus sp.
TKMIVTYTTRHVSNGYELLPSAVTTKPRVEIQGGDMRSFFT

SEQ ID NO: 274 - protein, 27 aa, Rubus sp.
TTRHVSNGYELLPSAVTTKPRVEIQGG

SEQ ID NO: 275 - protein, 26 aa, Rubus sp.
HLHWVVTDIPGTTDATFGREVVSYEM

SEQ ID NO: 276 - protein, 26 aa, Rubus sp.
LHWWVVTDIPGTTDATFGREVVSYEM